(19) United States
(12) Patent Application Publication
(10) Pub. No.: US 2018/0147271 A1
(43) Pub. Date: May 31, 2018

(54) ANTI-ROR1 CHIMERIC ANTIGEN RECEPTORS

(71) Applicants: bluebird bio, Inc., Cambridge, MA (US); Five Prime Therapeutics, Inc., South San Francisco, CA (US); Eureka Therapeutics, Inc., Emeryville, CA (US)

(72) Inventors: Richard MORGAN, Center Harbor, NH (US); Robert SIKORSKI, Woodside, CA (US); Brian WONG, Los Altos, CA (US); Emma MASTELLER, Redwood City, CA (US); Luis BORGES, San Mateo, CA (US); Cheng LIU, Emeryville, CA (US); Yiyang XU, Emeryville, CA (US)

(21) Appl. No.: 15/575,220

(22) PCT Filed: May 17, 2016
(86) PCT No.: PCT/US16/32906

§ 371 Date: Nov. 17, 2017

Related U.S. Application Data
(60) Provisional application No. 62/163,272, filed on May 18, 2015.
(60) Provisional application No. 62/193,514, filed on Jul. 16, 2015.
(60) Provisional application No. 62/307,928, filed on Mar. 14, 2016.
(60) Provisional application No. 62/322,414, filed on Apr. 14, 2016.

(51) Int. Cl. A61K 39/00; C07K 14/725; C07K 16/28; C07K 16/30; A61P 35/02

(52) U.S. Cl. CPC A61K 39/0011; C07K 14/7051; C07K 16/2803; C07K 16/30; C07K 2317/622; C07K 2317/73; C07K 2319/03; C07K 2319/74; A61P 35/02

(57) ABSTRACT

The invention provides improved compositions for adoptive cell therapies for cancers that express ROR1.

### CROSS REFERENCE TO RELATED APPLICATIONS

This application claims the benefit under 35 U.S.C. § 119(e) of U.S. Provisional Application No. 62/322,414, filed Apr. 14, 2016, U.S. Provisional Application No. 62/307,928, filed Mar. 14, 2016, U.S. Provisional Application No. 62/193,514, filed Jul. 16, 2015, and U.S. Provisional Application No. 62/163,272, filed May 18, 2015, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD_050_04WO_ST25.txt. The text file is 205 KB, was created on May 16, 2016, and is being submitted electronically via EFS-Web, concurrent with the filing of the specification.

### BACKGROUND

### Technical Field

The present invention relates to improved compositions and methods for treating cancer. More particularly, the invention relates to improved chimeric antigen receptors (CARs) comprising anti-receptor tyrosine kinase-like orphan receptor 1 (ROR1) antibodies or antigen binding fragments thereof, immune effector cells genetically modified to express these CARs, and use of these compositions to effectively treat ROR1 expressing cancers.

### Description of the Related Art

Receptor tyrosine kinase-like orphan receptor 1 (ROR1) is a member of the receptor tyrosine kinase family consisting of ROR1 and ROR2. RORs contain two distinct extracellular cysteine-rich domains and one transmembrane domain. Within the intracellular portion, ROR1 possesses a tyrosine kinase domain, two serine/threonine-rich domains and a proline-rich domain. The ROR1 gene encodes two well-defined isoforms: a short 393 amino acid (aa) intracellular protein (isoform 2) and a long 937 aa type-1 transmembrane protein (isoform 1). The long cell surface isoform is expressed on primary human B-chronic lymphocytic leukemias (B-CLL) and mantle cell lymphomas, a subset of B-acute lymphocytic leukemia, and many tumors, including those associated with a metastatic phenotype.

While ROR1 is selectively overexpressed in a number of solid and hematological malignancies; normal adult tissues lack significant ROR1 expression. ROR1 expression has been detected in normal adult adipocytes, pancreas, and lung, but at markedly lower levels than in tumor cells. In a primate model, targeting of ROR1 with high doses of adoptive immunotherapy showed no overt clinical toxicity of normal tissues expressing ROR1.

Thus, ROR1 represents a safe target for cancer-targeted immunotherapies.

### BRIEF SUMMARY

The invention generally provides improved vectors for generating T cell therapies and methods of using the same. More particularly, the invention provides anti-ROR1 CAR molecules and their use in treating, preventing, or ameliorating cancers that express ROR1.

In various embodiments, a chimeric antigen receptor (CAR) is provided comprising: an extracellular domain that comprises: a) an anti-receptor tyrosine kinase-like orphan receptor 1 (ROR1) antibody or antigen binding fragment thereof that binds one or more epitopes of a human ROR1 polypeptide, wherein the anti-ROR1 antibody or antigen binding fragment thereof comprises a variable light chain sequence comprising CDRL1-CDRL3 sequences set forth in SEQ ID NOs: 1-3, 9-11, 17-19, 25-27, 33-35, 41-43, 49-51, 57-59, 65-67, 73-75, 81-83, 89-91, 97-99, 105-107, 113-115, 121-123, 129-131, 137-139, 145-147, 153-155, 161-163, 169-171, 177-179, 185-187, 193-195, 201-203, 209-211, 217-219, 225-227, 233-235, 241-243, 249-251, 257-259, 265-267, 273-275, 281-283, 289-291, 297-299, 305-307, 313-315, 321-323, 329-331, 337-339, 345-347, or 353-355, and a variable heavy chain sequence comprising CDRH1-CDRH3 sequences set forth in SEQ ID NOs: 4-6, 12-14, 20-22, 28-30, 36-38, 44-46, 52-54, 60-62, 68-70, 76-78, 84-86, 92-94, 100-102, 108-110, 116-118, 124-126, 132-134, 140-142, 148-150, 156-158, 164-166, 172-174, 180-182, 188-190, 196-198, 204-206, 212-214, 220-222, 228-230, 236-238, 244-246, 252-254, 260-262, 268-270, 276-278, 284-286, 292-294, 300-302, 308-310, 316-318, 324-326, 332-334, 340-342, 348-350, or 356-358; b) a transmembrane domain; c) one or more intracellular co-stimulatory signaling domains; and d) a primary signaling domain.

In particular embodiments, the anti-ROR1 antibody or antigen binding fragment that binds the human ROR1 polypeptide is selected from the group consisting of: a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, Nanobody).

In certain embodiments, the anti-ROR1 antibody or antigen binding fragment that binds the human ROR1 polypeptide is an scFv.

In particular embodiments, the anti-ROR1 antibody or antigen binding fragment thereof comprises one or more light chain CDRs as set forth in any one of SEQ ID NOs: 1-3 and/or one or more heavy chain CDRs as set forth in any one of SEQ ID NOs: 4-6.

In some embodiments, the anti-ROR1 antibody or antigen binding fragment thereof comprises one or more light chain CDRs as set forth in any one of SEQ ID NOs: 9-11 and/or one or more heavy chain CDRs as set forth in any one of SEQ ID NOs: 12-14.

In additional embodiments, the anti-ROR1 antibody or antigen binding fragment thereof comprises one or more light chain CDRs as set forth in any one of SEQ ID NOs: 17-19 and/or one or more heavy chain CDRs as set forth in any one of SEQ ID NOs: 20-22.

In some embodiments, the anti-ROR1 antibody or antigen binding fragment thereof comprises one or more light chain CDRs as set forth in any one of SEQ ID NOs: 25-27 and/or one or more heavy chain CDRs as set forth in any one of SEQ ID NOs: 28-30.

In particular embodiments, the anti-ROR1 antibody or antigen binding fragment thereof comprises one or more light chain CDRs as set forth in any one of SEQ ID NOs: 33-35 and/or one or more heavy chain CDRs as set forth in any one of SEQ ID NOs: 36-38.

In particular embodiments, the anti-ROR1 antibody or antigen binding fragment thereof comprises one or more light chain CDRs as set forth in any one of SEQ ID NOs: 41-43 and/or one or more heavy chain CDRs as set forth in any one of SEQ ID NOs: 44-46.

In particular embodiments, the anti-ROR1 antibody or antigen binding fragment thereof comprises one or more light chain CDRs as set forth in any one of SEQ ID NOs: 49-51 and/or one or more heavy chain CDRs as set forth in any one of SEQ ID NOs: 52-54.

In further embodiments, the anti-ROR1 antibody or antigen binding fragment thereof comprises one or more light chain CDRs as set forth in any one of SEQ ID NOs: 57-59 and/or one or more heavy chain CDRs as set forth in any one of SEQ ID NOs: 60-62.

In certain embodiments, the anti-ROR1 antibody or antigen binding fragment thereof comprises one or more light chain CDRs as set forth in any one of SEQ ID NOs: 65-67 and/or one or more heavy chain CDRs as set forth in any one of SEQ ID NOs: 68-70.

In certain embodiments, the anti-ROR1 antibody or antigen binding fragment thereof comprises one or more light chain CDRs as set forth in any one of SEQ ID NOs: 73-75 and/or one or more heavy chain CDRs as set forth in any one of SEQ ID NOs: 76-78.

In some embodiments, the anti-ROR1 antibody or antigen binding fragment thereof comprises one or more light chain CDRs as set forth in any one of SEQ ID NOs: 81-83 and/or one or more heavy chain CDRs as set forth in any one of SEQ ID NOs: 84-86.

In particular embodiments, the anti-ROR1 antibody or antigen binding fragment thereof comprises one or more light chain CDRs as set forth in any one of SEQ ID NOs: 89-91 and/or one or more heavy chain CDRs as set forth in any one of SEQ ID NOs: 92-94.

In certain embodiments, the anti-ROR1 antibody or antigen binding fragment thereof comprises one or more light chain CDRs as set forth in any one of SEQ ID NOs: 97-99 and/or one or more heavy chain CDRs as set forth in any one of SEQ ID NOs: 100-102.

In further embodiments, the anti-ROR1 antibody or antigen binding fragment thereof comprises one or more light chain CDRs as set forth in any one of SEQ ID NOs: 105-107 and/or one or more heavy chain CDRs as set forth in any one of SEQ ID NOs: 108-110.

In further embodiments, the anti-ROR1 antibody or antigen binding fragment thereof comprises one or more light chain CDRs as set forth in any one of SEQ ID NOs: 113-115 and/or one or more heavy chain CDRs as set forth in any one of SEQ ID NOs: 116-118.

In some embodiments, the anti-ROR1 antibody or antigen binding fragment thereof comprises one or more light chain CDRs as set forth in any one of SEQ ID NOs: 121-123 and/or one or more heavy chain CDRs as set forth in any one of SEQ ID NOs: 124-126.

In particular embodiments, the anti-ROR1 antibody or antigen binding fragment thereof comprises one or more light chain CDRs as set forth in any one of SEQ ID NOs: 129-131 and/or one or more heavy chain CDRs as set forth in any one of SEQ ID NOs: 132-134.

In additional embodiments, the anti-ROR1 antibody or antigen binding fragment thereof comprises one or more light chain CDRs as set forth in any one of SEQ ID NOs: 137-139 and/or one or more heavy chain CDRs as set forth in any one of SEQ ID NOs: 140-142.

In particular embodiments, the anti-ROR1 antibody or antigen binding fragment thereof comprises one or more light chain CDRs as set forth in any one of SEQ ID NOs: 145-147 and/or one or more heavy chain CDRs as set forth in any one of SEQ ID NOs: 148-150.

In certain embodiments, the anti-ROR1 antibody or antigen binding fragment thereof comprises one or more light chain CDRs as set forth in any one of SEQ ID NOs: 153-155 and/or one or more heavy chain CDRs as set forth in any one of SEQ ID NOs: 156-158.

In some embodiments, the anti-ROR1 antibody or antigen binding fragment thereof comprises one or more light chain CDRs as set forth in any one of SEQ ID NOs: 161-163 and/or one or more heavy chain CDRs as set forth in any one of SEQ ID NOs: 164-166.

In further embodiments, the anti-ROR1 antibody or antigen binding fragment thereof comprises one or more light chain CDRs as set forth in any one of SEQ ID NOs: 169-171 and/or one or more heavy chain CDRs as set forth in any one of SEQ ID NOs: 172-174.

In particular embodiments, the anti-ROR1 antibody or antigen binding fragment thereof comprises one or more light chain CDRs as set forth in any one of SEQ ID NOs: 177-179 and/or one or more heavy chain CDRs as set forth in any one of SEQ ID NOs: 180-182.

In additional embodiments, the anti-ROR1 antibody or antigen binding fragment thereof comprises one or more light chain CDRs as set forth in any one of SEQ ID NOs: 185-187 and/or one or more heavy chain CDRs as set forth in any one of SEQ ID NOs: 188-190.

In some embodiments, the anti-ROR1 antibody or antigen binding fragment thereof comprises one or more light chain CDRs as set forth in any one of SEQ ID NOs: 193-195 and/or one or more heavy chain CDRs as set forth in any one of SEQ ID NOs: 196-198.

In particular embodiments, the anti-ROR1 antibody or antigen binding fragment thereof comprises one or more light chain CDRs as set forth in any one of SEQ ID NOs: 201-203 and/or one or more heavy chain CDRs as set forth in any one of SEQ ID NOs: 204-206.

In further embodiments, the anti-ROR1 antibody or antigen binding fragment thereof comprises one or more light chain CDRs as set forth in any one of SEQ ID NOs: 209-211 and/or one or more heavy chain CDRs as set forth in any one of SEQ ID NOs: 212-214.

In certain embodiments, the anti-ROR1 antibody or antigen binding fragment thereof comprises one or more light chain CDRs as set forth in any one of SEQ ID NOs: 217-219 and/or one or more heavy chain CDRs as set forth in any one of SEQ ID NOs: 220-222.

In further embodiments, the anti-ROR1 antibody or antigen binding fragment thereof comprises one or more light chain CDRs as set forth in any one of SEQ ID NOs: 225-227 and/or one or more heavy chain CDRs as set forth in any one of SEQ ID NOs: 228-230.

In some embodiments, the anti-ROR1 antibody or antigen binding fragment thereof comprises one or more light chain CDRs as set forth in any one of SEQ ID NOs: 233-235 and/or one or more heavy chain CDRs as set forth in any one of SEQ ID NOs: 236-238.

In some embodiments, the anti-ROR1 antibody or antigen binding fragment thereof comprises one or more light chain CDRs as set forth in any one of SEQ ID NOs: 241-243 and/or one or more heavy chain CDRs as set forth in any one of SEQ ID NOs: 244-246.

In particular embodiments, the anti-ROR1 antibody or antigen binding fragment thereof comprises one or more light chain CDRs as set forth in any one of SEQ ID NOs: 249-251 and/or one or more heavy chain CDRs as set forth in any one of SEQ ID NOs: 252-254.

In further embodiments, the anti-ROR1 antibody or antigen binding fragment thereof comprises one or more light chain CDRs as set forth in any one of SEQ ID NOs: 257-259 and/or one or more heavy chain CDRs as set forth in any one of SEQ ID NOs: 260-262.

In certain embodiments, the anti-ROR1 antibody or antigen binding fragment thereof comprises one or more light chain CDRs as set forth in any one of SEQ ID NOs: 265-267 and/or one or more heavy chain CDRs as set forth in any one of SEQ ID NOs: 268-270.

In additional embodiments, the anti-ROR1 antibody or antigen binding fragment thereof comprises one or more light chain CDRs as set forth in any one of SEQ ID NOs: 273-275 and/or one or more heavy chain CDRs as set forth in any one of SEQ ID NOs: 276-278.

In further embodiments, the anti-ROR1 antibody or antigen binding fragment thereof comprises one or more light chain CDRs as set forth in any one of SEQ ID NOs: 281-283 and/or one or more heavy chain CDRs as set forth in any one of SEQ ID NOs: 284-286.

In particular embodiments, the anti-ROR1 antibody or antigen binding fragment thereof comprises one or more light chain CDRs as set forth in any one of SEQ ID NOs: 289-291 and/or one or more heavy chain CDRs as set forth in any one of SEQ ID NOs: 292-294.

In particular embodiments, the anti-ROR1 antibody or antigen binding fragment thereof comprises one or more light chain CDRs as set forth in any one of SEQ ID NOs: 297-299 and/or one or more heavy chain CDRs as set forth in any one of SEQ ID NOs: 300-302.

In some embodiments, the anti-ROR1 antibody or antigen binding fragment thereof comprises one or more light chain CDRs as set forth in any one of SEQ ID NOs: 305-307 and/or one or more heavy chain CDRs as set forth in any one of SEQ ID NOs: 308-310.

In further embodiments, the anti-ROR1 antibody or antigen binding fragment thereof comprises one or more light chain CDRs as set forth in any one of SEQ ID NOs: 313-315 and/or one or more heavy chain CDRs as set forth in any one of SEQ ID NOs: 316-318.

In certain embodiments, the anti-ROR1 antibody or antigen binding fragment thereof comprises one or more light chain CDRs as set forth in any one of SEQ ID NOs: 321-323 and/or one or more heavy chain CDRs as set forth in any one of SEQ ID NOs: 324-326.

In particular embodiments, the anti-ROR1 antibody or antigen binding fragment thereof comprises one or more light chain CDRs as set forth in any one of SEQ ID NOs: 329-331 and/or one or more heavy chain CDRs as set forth in any one of SEQ ID NOs: 332-334.

In additional embodiments, the anti-ROR1 antibody or antigen binding fragment thereof comprises one or more light chain CDRs as set forth in any one of SEQ ID NOs: 337-339 and/or one or more heavy chain CDRs as set forth in any one of SEQ ID NOs: 340-342.

In further embodiments, the anti-ROR1 antibody or antigen binding fragment thereof comprises one or more light chain CDRs as set forth in any one of SEQ ID NOs: 345-347 and/or one or more heavy chain CDRs as set forth in any one of SEQ ID NOs: 348-350.

In some embodiments, the anti-ROR1 antibody or antigen binding fragment thereof comprises one or more light chain CDRs as set forth in any one of SEQ ID NOs: 353-355 and/or one or more heavy chain CDRs as set forth in any one of SEQ ID NOs: 356-358.

In some embodiments, the anti-ROR1 antibody or antigen binding fragment thereof comprises a variable light chain sequence as set forth in any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, 311, 319, 327, 335, 343, 351, or 359 and/or a variable heavy chain sequence as set forth in any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, 312, 320, 328, 336, 344, 352, and 360.

In certain embodiments, the anti-ROR1 antibody or antigen binding fragment thereof comprises a variable light chain sequence as set forth in SEQ ID NO: 7 and/or a variable heavy chain sequence as set forth in SEQ ID NO: 8.

In further embodiments, the anti-ROR1 antibody or antigen binding fragment thereof comprises a variable light chain sequence as set forth in SEQ ID NO: 15 and/or a variable heavy chain sequence as set forth in SEQ ID NO: 16.

In particular embodiments, the anti-ROR1 antibody or antigen binding fragment thereof comprises a variable light chain sequence as set forth in SEQ ID NO: 23 and/or a variable heavy chain sequence as set forth in SEQ ID NO: 24.

In additional embodiments, the anti-ROR1 antibody or antigen binding fragment thereof comprises a variable light chain sequence as set forth in SEQ ID NO: 31 and/or a variable heavy chain sequence as set forth in SEQ ID NO: 32.

In particular embodiments, the anti-ROR1 antibody or antigen binding fragment thereof comprises a variable light chain sequence as set forth in SEQ ID NO: 39 and/or a variable heavy chain sequence as set forth in SEQ ID NO: 40.

In some embodiments, the anti-ROR1 antibody or antigen binding fragment thereof comprises a variable light chain sequence as set forth in SEQ ID NO: 47 and/or a variable heavy chain sequence as set forth in SEQ ID NO: 48.

In particular embodiments, the anti-ROR1 antibody or antigen binding fragment thereof comprises a variable light chain sequence as set forth in SEQ ID NO: 55 and/or a variable heavy chain sequence as set forth in SEQ ID NO: 56.

In further embodiments, the anti-ROR1 antibody or antigen binding fragment thereof comprises a variable light chain sequence as set forth in SEQ ID NO: 63 and/or a variable heavy chain sequence as set forth in SEQ ID NO: 64.

In certain embodiments, the anti-ROR1 antibody or antigen binding fragment thereof comprises a variable light chain sequence as set forth in SEQ ID NO: 71 and/or a variable heavy chain sequence as set forth in SEQ ID NO: 72.

In additional embodiments, the anti-ROR1 antibody or antigen binding fragment thereof comprises a variable light chain sequence as set forth in SEQ ID NO: 79 and/or a variable heavy chain sequence as set forth in SEQ ID NO: 80.

In additional embodiments, the anti-ROR1 antibody or antigen binding fragment thereof comprises a variable light chain sequence as set forth in SEQ ID NO: 87 and/or a variable heavy chain sequence as set forth in SEQ ID NO: 88.

In some embodiments, the anti-ROR1 antibody or antigen binding fragment thereof comprises a variable light chain sequence as set forth in SEQ ID NO: 95 and/or a variable heavy chain sequence as set forth in SEQ ID NO: 96.

In particular embodiments, the anti-ROR1 antibody or antigen binding fragment thereof comprises a variable light chain sequence as set forth in SEQ ID NO: 103 and/or a variable heavy chain sequence as set forth in SEQ ID NO: 104.

In certain embodiments, the anti-ROR1 antibody or antigen binding fragment thereof comprises a variable light chain sequence as set forth in SEQ ID NO: 111 and/or a variable heavy chain sequence as set forth in SEQ ID NO: 112.

In particular embodiments, the anti-ROR1 antibody or antigen binding fragment thereof comprises a variable light chain sequence as set forth in SEQ ID NO: 119 and/or a variable heavy chain sequence as set forth in SEQ ID NO: 120.

In further embodiments, the anti-ROR1 antibody or antigen binding fragment thereof comprises a variable light chain sequence as set forth in SEQ ID NO: 127 and/or a variable heavy chain sequence as set forth in SEQ ID NO: 128.

In certain embodiments, the anti-ROR1 antibody or antigen binding fragment thereof comprises a variable light chain sequence as set forth in SEQ ID NO: 135 and/or a variable heavy chain sequence as set forth in SEQ ID NO: 136.

In some embodiments, the anti-ROR1 antibody or antigen binding fragment thereof comprises a variable light chain sequence as set forth in SEQ ID NO: 143 and/or a variable heavy chain sequence as set forth in SEQ ID NO: 144.

In particular embodiments, the anti-ROR1 antibody or antigen binding fragment thereof comprises a variable light chain sequence as set forth in SEQ ID NO: 151 and/or a variable heavy chain sequence as set forth in SEQ ID NO: 152.

In additional embodiments, the anti-ROR1 antibody or antigen binding fragment thereof comprises a variable light chain sequence as set forth in SEQ ID NO: 159 and/or a variable heavy chain sequence as set forth in SEQ ID NO: 160.

In further embodiments, the anti-ROR1 antibody or antigen binding fragment thereof comprises a variable light chain sequence as set forth in SEQ ID NO: 167 and/or a variable heavy chain sequence as set forth in SEQ ID NO: 168.

In particular embodiments, the anti-ROR1 antibody or antigen binding fragment thereof comprises a variable light chain sequence as set forth in SEQ ID NO: 175 and/or a variable heavy chain sequence as set forth in SEQ ID NO: 176.

In some embodiments, the anti-ROR1 antibody or antigen binding fragment thereof comprises a variable light chain sequence as set forth in SEQ ID NO: 183 and/or a variable heavy chain sequence as set forth in SEQ ID NO: 184.

In certain embodiments, the anti-ROR1 antibody or antigen binding fragment thereof comprises a variable light chain sequence as set forth in SEQ ID NO: 191 and/or a variable heavy chain sequence as set forth in SEQ ID NO: 192.

In additional embodiments, the anti-ROR1 antibody or antigen binding fragment thereof comprises a variable light chain sequence as set forth in SEQ ID NO: 199 and/or a variable heavy chain sequence as set forth in SEQ ID NO: 200.

In particular embodiments, the anti-ROR1 antibody or antigen binding fragment thereof comprises a variable light chain sequence as set forth in SEQ ID NO: 207 and/or a variable heavy chain sequence as set forth in SEQ ID NO: 208.

In further embodiments, the anti-ROR1 antibody or antigen binding fragment thereof comprises a variable light chain sequence as set forth in SEQ ID NO: 215 and/or a variable heavy chain sequence as set forth in SEQ ID NO: 216.

In some embodiments, the anti-ROR1 antibody or antigen binding fragment thereof comprises a variable light chain sequence as set forth in SEQ ID NO: 223 and/or a variable heavy chain sequence as set forth in SEQ ID NO: 224.

In particular embodiments, the anti-ROR1 antibody or antigen binding fragment thereof comprises a variable light chain sequence as set forth in SEQ ID NO: 231 and/or a variable heavy chain sequence as set forth in SEQ ID NO: 232.

In further embodiments, the anti-ROR1 antibody or antigen binding fragment thereof comprises a variable light chain sequence as set forth in SEQ ID NO: 239 and/or a variable heavy chain sequence as set forth in SEQ ID NO: 240.

In additional embodiments, the anti-ROR1 antibody or antigen binding fragment thereof comprises a variable light chain sequence as set forth in SEQ ID NO: 247 and/or a variable heavy chain sequence as set forth in SEQ ID NO: 248.

In certain embodiments, the anti-ROR1 antibody or antigen binding fragment thereof comprises a variable light chain sequence as set forth in SEQ ID NO: 255 and/or a variable heavy chain sequence as set forth in SEQ ID NO: 256.

In particular embodiments, the anti-ROR1 antibody or antigen binding fragment thereof comprises a variable light chain sequence as set forth in SEQ ID NO: 263 and/or a variable heavy chain sequence as set forth in SEQ ID NO: 264.

In further embodiments, the anti-ROR1 antibody or antigen binding fragment thereof comprises a variable light chain sequence as set forth in SEQ ID NO: 271 and/or a variable heavy chain sequence as set forth in SEQ ID NO: 272.

In some embodiments, the anti-ROR1 antibody or antigen binding fragment thereof comprises a variable light chain sequence as set forth in SEQ ID NO: 279 and/or a variable heavy chain sequence as set forth in SEQ ID NO: 280.

In further embodiments, the anti-ROR1 antibody or antigen binding fragment thereof comprises a variable light chain sequence as set forth in SEQ ID NO: 287 and/or a variable heavy chain sequence as set forth in SEQ ID NO: 288.

In particular embodiments, the anti-ROR1 antibody or antigen binding fragment thereof comprises a variable light chain sequence as set forth in SEQ ID NO: 295 and/or a variable heavy chain sequence as set forth in SEQ ID NO: 296.

In certain embodiments, the anti-ROR1 antibody or antigen binding fragment thereof comprises a variable light chain sequence as set forth in SEQ ID NO: 303 and/or a variable heavy chain sequence as set forth in SEQ ID NO: 304.

In additional embodiments, the anti-ROR1 antibody or antigen binding fragment thereof comprises a variable light chain sequence as set forth in SEQ ID NO: 311 and/or a variable heavy chain sequence as set forth in SEQ ID NO: 312.

In particular embodiments, the anti-ROR1 antibody or antigen binding fragment thereof comprises a variable light chain sequence as set forth in SEQ ID NO: 319 and/or a variable heavy chain sequence as set forth in SEQ ID NO: 320.

In particular embodiments, the anti-ROR1 antibody or antigen binding fragment thereof comprises a variable light chain sequence as set forth in SEQ ID NO: 327 and/or a variable heavy chain sequence as set forth in SEQ ID NO: 328.

In particular embodiments, the anti-ROR1 antibody or antigen binding fragment thereof comprises a variable light chain sequence as set forth in SEQ ID NO: 335 and/or a variable heavy chain sequence as set forth in SEQ ID NO: 336.

In certain embodiments, the anti-ROR1 antibody or antigen binding fragment thereof comprises a variable light chain sequence as set forth in SEQ ID NO: 343 and/or a variable heavy chain sequence as set forth in SEQ ID NO: 344.

In some embodiments, the anti-ROR1 antibody or antigen binding fragment thereof comprises a variable light chain sequence as set forth in SEQ ID NO: 351 and/or a variable heavy chain sequence as set forth in SEQ ID NO: 352.

In additional embodiments, the anti-ROR1 antibody or antigen binding fragment thereof comprises a variable light chain sequence as set forth in SEQ ID NO: 359 and/or a variable heavy chain sequence as set forth in SEQ ID NO: 360.

In further embodiments, the transmembrane domain is from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1.

In additional embodiments, the transmembrane domain is from a polypeptide selected from the group consisting of: CD8α; CD4, CD45, PD1, and CD152.

In some embodiments, the transmembrane domain is from CD8α.

In further embodiments, the transmembrane domain is from PD1.

In particular embodiments, the transmembrane domain is from CD152.

In further embodiments, the one or more co-stimulatory signaling domains are from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

In certain embodiments, the one or more co-stimulatory signaling domains are from a co-stimulatory molecule selected from the group consisting of: CD28, CD134, and CD137.

In some embodiments, the one or more co-stimulatory signaling domains is from CD28.

In some embodiments, the one or more co-stimulatory signaling domains is from CD134.

In some embodiments, the one or more co-stimulatory signaling domains is from CD137.

In particular embodiments, the primary signaling domain is isolated from a polypeptide selected from the group consisting of: FcRγ, FcRβ, CD3γ, CD3δ, CD3ε, CD3ζ, CD22, CD79a, CD79b, and CD66d.

In particular embodiments, the primary signaling domain is isolated from CD3.

In additional embodiments, the CAR further comprises a hinge region polypeptide.

In certain embodiments, the hinge region polypeptide comprises a hinge region of CD8α.

In further embodiments, the hinge region polypeptide comprises a hinge region of PD1.

In particular embodiments, the hinge region polypeptide comprises a hinge region of CD152.

In additional embodiments, the CAR further comprises a spacer region.

In further embodiments, the spacer region polypeptide comprises CH2 and CH3 regions of IgG1, IgG4, or IgD.

In further embodiments, the CAR further comprises a signal peptide.

In particular embodiments, the signal peptide comprises an IgG1 heavy chain signal polypeptide, a CD8α signal polypeptide, or a human GM-CSF receptor alpha signal polypeptide.

In various embodiments, a polypeptide comprising the amino acid sequence of a CAR contemplated herein is provided.

In particular embodiments, a CAR comprises an amino acid sequence set forth in any one of SEQ ID NOs: 386-397.

In particular embodiments, a CAR comprises an amino acid sequence set forth in SEQ ID NO: 386.

In particular embodiments, a CAR comprises an amino acid sequence set forth in SEQ ID NO: 387.

In particular embodiments, a CAR comprises an amino acid sequence set forth in SEQ ID NO: 388.

In particular embodiments, a CAR comprises an amino acid sequence set forth in SEQ ID NO: 389.

In particular embodiments, a CAR comprises an amino acid sequence set forth in SEQ ID NO: 390.

In particular embodiments, a CAR comprises an amino acid sequence set forth in SEQ ID NO: 391.

In particular embodiments, a CAR comprises an amino acid sequence set forth in SEQ ID NO: 392.

In particular embodiments, a CAR comprises an amino acid sequence set forth in SEQ ID NO: 393.

In particular embodiments, a CAR comprises an amino acid sequence set forth in SEQ ID NO: 394.

In particular embodiments, a CAR comprises an amino acid sequence set forth in SEQ ID NO: 395.

In particular embodiments, a CAR comprises an amino acid sequence set forth in SEQ ID NO: 396.

In particular embodiments, a CAR comprises an amino acid sequence set forth in SEQ ID NO: 397.

In various embodiments, a polynucleotide encoding a CAR contemplated herein is provided.

In various embodiments, a vector comprising a polynucleotide encoding a CAR contemplated herein is provided.

In certain embodiments, the vector is an expression vector.

In particular embodiments, the vector is an episomal vector.

In further embodiments, the vector is a viral vector.

In further embodiments, the vector is a retroviral vector.

In particular embodiments, the vector is a lentiviral vector.

In further embodiments, the lentiviral vector is selected from the group consisting essentially of: human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus (SIV).

In particular embodiments, the vector comprises a left (5′) retroviral LTR, a Psi (Ψ) packaging signal, a central polypurine tract/DNA flap (cPPT/FLAP), a retroviral export element; a promoter operably linked to the polynucleotide; and a right (3′) retroviral LTR.

In further embodiments, the vector further comprises a heterologous polyadenylation sequence.

In particular embodiments, the vector further comprises a hepatitis B virus post-transcriptional regulatory element (HPRE) or woodchuck post-transcriptional regulatory element (WPRE).

In additional embodiments, the promoter of the 5′ LTR is replaced with a heterologous promoter.

In further embodiments, the heterologous promoter is a cytomegalovirus (CMV) promoter, a Rous Sarcoma Virus (RSV) promoter, or an Simian Virus 40 (SV40) promoter.

In some embodiments, the 5′ LTR or 3′ LTR is a lentivirus LTR.

In certain embodiments, the 3′ LTR comprises one or more modifications.

In certain embodiments, the 3′ LTR comprises one or more deletions.

In particular embodiments, the 3′ LTR is a self-inactivating (SIN) LTR.

In particular embodiments, the polyadenylation sequence is a bovine growth hormone polyadenylation or signal rabbit β-globin polyadenylation sequence.

In additional embodiments, the polynucleotide comprises an optimized Kozak sequence.

In additional embodiments, the promoter operably linked to the polynucleotide is selected from the group consisting of: a cytomegalovirus immediate early gene promoter (CMV), an elongation factor 1 alpha promoter (EF1-α), a phosphoglycerate kinase-1 promoter (PGK), a ubiquitin-C promoter (UBQ-C), a cytomegalovirus enhancer/chicken beta-actin promoter (CAG), polyoma enhancer/herpes simplex thymidine kinase promoter (MCI), a beta actin promoter (β-ACT), a simian virus 40 promoter (SV40), and a myeloproliferative sarcoma virus enhancer, negative control region deleted, d1587rev primer-binding site substituted (MND) promoter.

In various embodiments, an immune effector cell comprising a vector encoding a CAR contemplated herein is provided.

In particular embodiments, the immune effector cell is selected from the group consisting of: a T lymphocyte and a natural killer (NK) cell.

In some embodiments, the immune effector cell is transduced with a vector contemplated herein and is activated and stimulated in the presence of an inhibitor of the PI3K pathway, thereby maintaining proliferation of the transduced immune effector cells compared to the proliferation of transduced immune effector cells that were activated and stimulated in the absence of the inhibitor of the PI3K pathway.

In particular embodiments, the immune effector cell activated and stimulated in the presence of the inhibitor of PI3K pathway has increased expression of i) one or more markers selected from the group consisting of: CD62L, CD127, CD197, and CD38 or ii) all of the markers CD62L, CD127, CD197, and CD38 compared to an immune effector cell activated and stimulated in the absence of the inhibitor of PI3K pathway.

In particular embodiments, the immune effector cell activated and stimulated in the presence of the inhibitor of PI3K pathway has increased expression of i) one or more markers selected from the group consisting of: CD62L, CD127, CD27, and CD8 or ii) all of the markers CD62L, CD127, CD27, and CD8 compared to an immune effector cell activated and stimulated in the absence of the inhibitor of PI3K pathway.

In one embodiment, the PI3K inhibitor is ZSTK474.

In various embodiments, a composition is provided comprising the immune effector cell contemplated herein and a physiologically acceptable excipient.

In various embodiments, a method of generating an immune effector cell comprising a CAR contemplated herein is provided comprising introducing into an immune effector cell a vector encoding a CAR contemplated herein.

In particular embodiments, the method further comprises stimulating the immune effector cell and inducing the cell to proliferate by contacting the cell with antibodies that bind CD3 and antibodies that bind to CD28; thereby generating a population of immune effector cells.

In certain embodiments, the immune effector cell is stimulated and induced to proliferate before introducing the vector.

In additional embodiments, the immune effector cells comprise T lymphocytes.

In some embodiments, the immune effector cells comprise NK cells.

In particular embodiments, the cells are the activated and stimulated in the presence of an inhibitor of the PI3K pathway, thereby maintaining proliferation of the transduced immune effector cells compared to the proliferation of immune effector cells that are activated and stimulated in the absence of the inhibitor of the PI3K pathway.

In some embodiments, the immune effector cells activated and stimulated in the presence of the inhibitor of PI3K pathway have increased expression of i) one or more markers selected from the group consisting of: CD62L, CD127, CD197, and CD38 or ii) all of the markers CD62L, CD127, CD197, and CD38 compared to immune effector cells activated and stimulated in the absence of the inhibitor of PI3K pathway.

In particular embodiments, the immune effector cell activated and stimulated in the presence of the inhibitor of PI3K pathway has increased expression of i) one or more markers selected from the group consisting of: CD62L, CD127, CD27, and CD8 or ii) all of the markers CD62L, CD127, CD27, and CD8 compared to an immune effector cell activated and stimulated in the absence of the inhibitor of PI3K pathway.

In one embodiment, the PI3K inhibitor is ZSTK474.

In various embodiments, method for increasing the cytotoxicity in cancer cells that express ROR1 in a subject is provided, comprising administering to the subject an amount of a composition contemplated herein sufficient to increase the cytotoxicity in cancer cells that express ROR1 compared to the cytotoxicity of the cancer cells that express ROR1 prior to the administration.

In various embodiments, a method for decreasing the number of cancer cells expressing ROR1 in a subject is provided, comprising administering to the subject an amount of a composition contemplated herein sufficient to decrease the number of cancer cells that express ROR1 compared to the number of the cancer cells that express ROR1 prior to the administration.

In various embodiments, a method of treating a cancer in a subject in need thereof, is provided comprising administering to the subject a therapeutically effect amount of a composition contemplated herein.

In particular embodiments, the cancer is a solid cancer.

In certain embodiments, the cancer is a liquid cancer.

In some embodiments, the cancer is a hematological malignancy.

In further embodiments, the cancer is the cancer is lung cancer, breast cancer, pancreatic cancer, ovarian cancer, prostate cancer, adrenal cancer, melanoma, uterine cancer, testicular cancer, or bladder cancer, non-Hodgkin's lymphoma, acute lymphocytic leukemia (ALL), chronic lymphocytic leukemia (CLL), hairy cell leukemia (HCL), multiple myeloma (MM), acute myeloid leukemia (AML), or chronic myeloid leukemia (CIVIL).

In particular embodiments, the non-Hodgkin's lymphoma is small lymphocytic lymphoma (SLL), diffuse large B cell lymphoma (DLBCL), follicular lymphoma (FL), mantle cell lymphoma (MCL), or marginal zone lymphoma (MZL).

In additional embodiments, the cancer is the cancer is lung cancer, breast cancer, pancreatic cancer, ovarian cancer, prostate cancer, adrenal cancer, melanoma, uterine cancer, testicular cancer, or bladder cancer.

In some embodiments, the cancer is small lymphocytic lymphoma (SLL), diffuse large B cell lymphoma (DLBCL), follicular lymphoma (FL), mantle cell lymphoma (MCL), or marginal zone lymphoma (MZL).

In certain embodiments, the cancer is acute lymphocytic leukemia (ALL), chronic lymphocytic leukemia (CLL), hairy cell leukemia (HCL), multiple myeloma (MM), acute myeloid leukemia (AML), or chronic myeloid leukemia (CIVIL).

In various embodiments, a method for ameliorating at one or more symptoms associated with a cancer expressing ROR1 in a subject is provided, comprising administering to the subject an amount of a composition contemplated herein sufficient to ameliorate at least one symptom associated with cancer cells that express ROR1.

In particular embodiments, the one or more symptoms ameliorated are selected from the group consisting of: weakness, fatigue, shortness of breath, easy bruising and bleeding, frequent infections, enlarged lymph nodes, distended or painful abdomen, bone or joint pain, fractures, unplanned weight loss, poor appetite, night sweats, persistent mild fever, and decreased urination.

### BRIEF DESCRIPTION OF SEVERAL VIEWS OF THE DRAWINGS

FIG. 1 shows a schematic of anti-ROR1 CAR constructs.

FIG. 2A shows the vector copy number (VCN) from three primary T cell cultures transduced with lentivirus encoding an anti-ROR1 CARs.

FIG. 2B shows representative anti-ROR1 CAR expression on T cells measured using flow cytometry.

FIG. 2C shows representative anti-ROR1 CAR expression on T cells measured using flow cytometry.

FIG. 3 shows that anti-ROR1 CAR T cells co cultured with K562 (ROR1⁻), MCF7 (ROR1⁻), A549 (ROR1⁺), or K562 (ROR1⁺) cell lines for 24 hours released IFNγ only in the presence of ROR1 positive cell lines.

FIG. 4A shows that anti-ROR1 CAR T cells cause cytotoxicity of ROR1⁺ target cells during co-culture.

FIG. 4B shows that anti-ROR1 CAR T cells cause cytotoxicity of ROR1⁺ target cells during co-culture.

FIG. 5 shows that anti-ROR1 CAR T cells delay tumor growth in a mouse model of non-small cell lung cancer (NSCLC).

FIG. 6 shows representative anti-ROR1 CAR expression measured using flow cytometry and the vector copy number (VCN) from three primary T cell cultures transduced with lentivirus encoding an anti-ROR1 CARs.

FIG. 7 shows that anti-ROR1 CAR T cells co cultured with T cells alone, K562 (ROR1⁻), MCF7 (ROR1⁻), A549 (ROR1⁻), or NCI-H1915 (ROR1⁻) cell lines for 24 hours released IFNγ only in the presence of ROR1 positive cell lines.

FIG. 8 shows representative anti-ROR1 CAR vector copy number (VCN) from three primary T cell cultures transduced with lentivirus encoding an anti-ROR1 CARs.

FIG. 9 shows representative anti-ROR1 CAR expression measured using flow cytometry from three primary T cell cultures transduced with lentivirus encoding an anti-ROR1 CARs.

FIG. 10 shows that anti-ROR1 CAR T cells co cultured with T cells alone, K562 (ROR1⁻; upper left panel), MCF7 (ROR1⁻; upper right panel), A549 (ROR1⁺; lower left panel), or NCI-H1915 (ROR1⁺; lower right panel) cell lines for 24 hours released IFNγ only in the presence of ROR1 positive cell lines.

FIG. 11 shows antigen-specific cytotoxicity of ROR1⁺ suspension RPMI-8226 tumor cells co-cultured with anti-ROR1 CAR T cells at various effector:target cell ratios after four hours of co-culture.

FIG. 12A shows cytotoxicity of ROR1⁺ A549 adherent tumor cells co-cultured with anti-ROR1 CAR T cells monitored in real time with an iCELLigence instrument.

FIG. 12B shows dose-dependent cytotoxicity of ROR1⁺ A549 adherent tumor cells co-cultured with anti-ROR1 CAR T cells at 10 hours measured with an iCELLigence instrument.

### BRIEF DESCRIPTION OF THE SEQUENCE IDENTIFIERS

SEQ ID NOs: 1-360 set forth amino acid sequences of exemplary light chain CDR sequences, heavy chain CDR sequences, variable domain light chains, and variable domain heavy chains for anti-ROR1 CARs contemplated herein.

SEQ ID NO: 361 sets forth the amino acid sequence of human ROR1.

SEQ ID NOs: 362-372 set for the amino acid sequences of various linkers.

SEQ ID NOs: 373-385 set for the amino acid sequences of protease cleavage sites and self-cleaving polypeptide cleavage sites.

SEQ ID NOs: 386-397 set forth the amino acid sequences of exemplary anti-ROR1 CARs.

### DETAILED DESCRIPTION

### A. Overview

The invention generally relates to improved compositions and methods for preventing or treating cancers that express ROR1 or preventing, treating, or ameliorating at least one symptom associated with an ROR1 expressing cancer. In particular embodiments, the invention relates to improved adoptive cell therapy of cancers that express ROR1 using genetically modified immune effector cells. Genetic approaches offer a potential means to enhance immune recognition and elimination of cancer cells. One promising strategy is to genetically engineer immune effector cells to express chimeric antigen receptors (CAR) that redirect cytotoxicity toward cancer cells.

The improved compositions and methods of adoptive cell therapy contemplated herein, provide genetically modified immune effector cells that can readily be expanded, exhibit long-term persistence in vivo, and demonstrate antigen dependent cytotoxicity to cells expressing receptor tyrosine kinase-like orphan receptor 1 (ROR1, also known as neurotrophic tyrosine kinase, receptor-related 1; NTRKR1).

ROR1 is a transmembrane protein within the receptor tyrosine kinase (RTK) family and is closely related to MUSK and Trk family receptors (Masiakowski and Carroll, 1992; Forrester et al., 1999). The structure of human ROR1 consists of an extracellular immunoglobulin-like (Ig) domain at the amino-terminus, a Frizzled domain (FZD), a Kringle domain (KRD), a transmembrane domain, a tyrosine kinase domain (TKD), a Serine/Threonine-rich domain (Ser/Thr), a proline-rich domain (PRD), and a second Ser/Thr domain at the carboxy-terminus. The ROR family of proteins is evolutionally conserved and shares a high level of homology between orthologs in Mus musculus, Caenorhabditis elegans, Xenopus laevis, Drosophila melanogaster, Aplysia californica, and Gallus gallus (Wilson et al., 1993; Forrester et al., 1999; Oishi et al., 1999; McKay et al., 2001; Hikasa et al., 2002; Stricker et al., 2006). The conservation of RORs across species underlies the importance of the ROR family through a number of processes during evolution.

ROR1 expression is present during normal embryonic and fetal development, it is absent within most mature tissues. A low level of ROR1 expression is seen in adipose tissue and to a lesser degree in the pancreas, lung, and a subset of intermediate B cells (Baskar et al., 2008; Hudecek et al., 2010; Bicocca et al., 2012). A growing literature has established ROR1 as a marker for cancer, including various solid tumors and hematological malignancies. In addition, ROR1 is involved in progression of a number of blood and solid malignancies. ROR1 has also been shown to inhibit apoptosis, potentiate EGFR signaling, and induce epithelial-mesenchymal transition (EMT). Because ROR1 is generally absent in adult tissue, is expressed in various cancers, and plays a role in various pathways of oncogenesis; it represents a potential drug target for cancer therapy.

Strong expression of ROR1 was initially identified in B-Cell chronic lymphocytic leukemia (CLL) (Baskar et al., 2008). Since the discovery of the elevated expression of ROR1 in CLL, increased levels of ROR1 have been described in a variety of hematological malignancies, including acute lymphocytic leukemia (ALL), non-Hodgkin lymphomas (NHL), and myeloid malignancies (Baskar et al., 2008; Daneshmanesh et al., 2008; Barna et al., 2011; Daneshmanesh et al., 2013). Specifically for NHLs, when compared to PBMCs, ROR1 mRNA and/or protein are elevated in all or a subset of primary samples of mantle cell lymphoma (MCL), marginal zone lymphoma (MZL), diffuse large B-cell lymphoma (DLBCL), and follicular lymphoma (Barna et al., 2011; Daneshmanesh et al., 2013).

ROR1 is also highly expressed in a wide variety of solid tumors. Tissue microarray analysis showed expression of ROR1 in primary samples in colon, lung, pancreatic, ovarian, lymphoma, skin, testicular, uterine, prostate, and adrenal cancers (Zhang et al., 2012b). ROR1 is also expressed in human neoplastic breast cancer cells, and absent in stromal cells (Zhang et al., 2012a; Cui et al., 2013). In addition, ROR1 mRNA can be detected in 81.3% of tissue samples and 94% of PBMCs samples from renal cancer patients as determined by RT-PCR (Rabbani et al., 2010). Furthermore, PBMCs from renal cancer patients showed significantly higher ROR1 expression, compared to healthy controls. Furthermore, high expression of ROR1 is associated with higher grade and more aggressive disease.

In various embodiments, CARs comprising anti-ROR1 antibody sequences are highly efficacious; undergo robust in vivo expansion; and recognize cancer cells expressing ROR1 and show cytotoxic activity against the ROR1 expressing cancer cells.

In one embodiment, a CAR comprising an anti-ROR1 antibody or antigen binding fragment, a transmembrane domain, and one or more intracellular signaling domains is provided.

In one embodiment, an immune effector cell is genetically modified to express a CAR. T cells expressing a CAR are referred to herein as CAR T cells or CAR modified T cells.

In various embodiments, genetically modified immune effector cells are administered to a patient with cancer cells expressing ROR1 including, but not limited to solid tumors and hematological malignancies.

The practice of particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

### C. Chimeric Antigen Receptors

In various embodiments, genetically engineered receptors that redirect cytotoxicity of immune effector cells toward cancer cells expressing ROR1 are provided. These genetically engineered receptors referred to herein as chimeric antigen receptors (CARs). CARs are molecules that combine antibody-based specificity for a desired antigen (e.g., ROR1) with a T cell receptor-activating intracellular domain to generate a chimeric protein that exhibits a specific anti-ROR1 cellular immune activity. As used herein, the term, “chimeric,” describes being composed of parts of different proteins or DNAs from different origins.

In particular embodiments, CARs comprise an extracellular domain (also referred to as a binding domain or antigen-specific binding domain) that binds to ROR1, a transmembrane domain, and an intracellular signaling domain. Engagement of the anti-ROR1 antigen binding domain of the CAR with ROR1 on the surface of a target cell results in clustering of the CAR and delivers an activation stimulus to the CAR-containing cell. The main characteristic of CARs are their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors.

In various embodiments, a CAR comprises an extracellular binding domain that comprises an ROR1-specific binding domain; a transmembrane domain; one or more intracellular co-stimulatory signaling domains; and a primary signaling domain.

In particular embodiments, a CAR comprises an extracellular binding domain that comprises an anti-ROR1 antibody or antigen binding fragment thereof; one or more hinge domains or spacer domains; a transmembrane domain including; one or more intracellular co-stimulatory signaling domains; and a primary signaling domain.

1. Binding Domain

In particular embodiments, CARs comprise an extracellular binding domain that comprises an anti-ROR1 antibody or antigen binding fragment thereof that specifically binds to a human ROR1 polypeptide expressed on a target cell, e.g., a cancer cell. As used herein, the terms, “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., ROR1. The binding domain may be derived either from a natural, synthetic, semi-synthetic, or recombinant source.

The terms “specific binding affinity” or “specifically binds” or “specifically bound” or “specific binding” or “specifically targets” as used herein, describe binding of an anti-ROR1 antibody or antigen binding fragment thereof (or a CAR comprising the same) to ROR1 at greater binding affinity than background binding. A binding domain (or a CAR comprising a binding domain or a fusion protein containing a binding domain) “specifically binds” to an ROR1 polypeptide if it binds to or associates with ROR1 with an affinity or Kₐ (i.e., an equilibrium association constant of a particular binding interaction with units of 1/M) of, for example, greater than or equal to about 10⁵ M⁻¹. In certain embodiments, a binding domain (or a fusion protein thereof) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains (or single chain fusion proteins thereof) refers to those binding domains with a Kₐ of at least 10⁷ M⁻¹, at least 10⁸M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (K_{d}) of a particular binding interaction with units of M (e.g., 10⁻⁵ M to 10⁻¹³M, or less). Affinities of binding domain polypeptides and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, N.J., or optical biosensor technology such as the EPIC system or EnSpire that are available from Corning and Perkin Elmer respectively (see also, e.g., Scatchard et al. (1949) Ann. N.Y. Acad. Sci. 51:660; and U.S. Pat. Nos. 5,283,173; 5,468,614, or the equivalent).

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

In particular embodiments, the extracellular binding domain of a CAR comprises an antibody or antigen binding fragment thereof. An “antibody” refers to a binding agent that is a polypeptide comprising at least a light chain or heavy chain immunoglobulin variable region which specifically recognizes and binds an epitope of an antigen, such as a peptide, lipid, polysaccharide, or nucleic acid containing an antigenic determinant, such as those recognized by an immune cell.

An “antigen (Ag)” refers to a compound, composition, or substance that can stimulate the production of antibodies or a T cell response in an animal, including compositions (such as one that includes a cancer-specific protein) that are injected or absorbed into an animal. An antigen reacts with the products of specific humoral or cellular immunity, including those induced by heterologous antigens, such as the disclosed antigens. In particular embodiments, the target antigen is an epitope of an ROR1 polypeptide.

An “epitope” or “antigenic determinant” refers to the region of an antigen to which a binding agent binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation.

Antibodies include antigen binding fragments thereof, such as Camel Ig, Ig NAR, Fab fragments, Fab′ fragments, F(ab)′2 fragments, F(ab)′3 fragments, Fv, single chain Fv proteins (“scFv”), bis-scFv, (scFv)₂, minibodies, diabodies, triabodies, tetrabodies, disulfide stabilized Fv proteins (“dsFv”), and single-domain antibody (sdAb, Nanobody) and portions of full length antibodies responsible for antigen binding. The term also includes genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, Ill.); Kuby, J., Immunology, 3rd Ed., W. H. Freeman & Co., New York, 1997.

As would be understood by the skilled person and as described elsewhere herein, a complete antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ. Mammalian light chains are classified as λ or κ. Immunoglobulins comprising the α, δ, ε, γ, and μ heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains μ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity-determining regions” or “CDRs.” The CDRs can be defined or identified by conventional methods, such as by sequence according to Kabat et al. (Wu, T T and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Chothia, C. and Lesk, A. M., J Mol. Biol., 196(4): 901-917 (1987), Chothia, C. et al, Nature, 342: 877-883 (1989)).

Illustrative examples of rules for predicting light chain CDRs include: CDR-L1 starts at about residue 24, is preceded by a Cys, is about 10-17 residues, and is followed by a Trp (typically Trp-Tyr-Gln, but also, Trp-Leu-Gln, Trp-Phe-Gln, Trp-Tyr-Leu); CDR-L2 starts about 16 residues after the end of CDR-L1, is generally preceded by Ile-Tyr, but also, Val-Tyr, Ile-Lys, Ile-Phe, and is 7 residues; and CDR-L3 starts about 33 residues after the end of CDR-L2, is preceded by a Cys, is 7-11 residues, and is followed by Phe-Gly-XXX-Gly (SEQ ID NO:398; XXX is any amino acid).

Illustrative examples of rules for predicting heavy chain CDRs include: CDR-H1 starts at about residue 26, is preceded by Cys-XXX-XXX-XXX (SEQ ID NO:399), is 10-12 residues and is followed by a Trp (typically Trp-Val, but also, Trp-Ile, Trp-Ala); CDR-H2 starts about 15 residues after the end of CDR-H1, is generally preceded by Leu-Glu-Trp-Ile-Gly (SEQ ID NO:400), or a number of variations, is 16-19 residues, and is followed by Lys/Arg-Leu/Ile/Val/Phe/Thr/Ala-Thr/Ser/Ile/Ala; and CDR-H3 starts about 33 residues after the end of CDR-H2, is preceded by Cys-XXX-XXX (typically Cys-Ala-Arg), is 3 to 25 residues, and is followed by Trp-Gly-XXX-Gly (SEQ ID NO:401).

In one embodiment, light chain CDRs and the heavy chain CDRs are determined according to the Kabat method.

In one embodiment, light chain CDRs and the heavy chain CDR2 and CDR3 are determined according to the Kabat method, and heavy chain CDR1 is determined according to the AbM method, which is a comprise between the Kabat and Chothia methods, see e.g., Whitelegg N & Rees A R, Protein Eng. 2000 December; 13(12):819-24 and Methods Mol Biol. 2004; 248:51-91. Programs for predicting CDRs are publicly available, e.g., AbYsis (www.bioinforg.uk/abysis/).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). Illustrative examples of light chain CDRs that are suitable for constructing anti-ROR1 CARs contemplated in particular embodiments include, but are not limited to the CDR sequences set forth in SEQ ID NOs: 1-3, 9-11, 17-19, 25-27, 33-35, 41-43, 49-51, 57-59, 65-67, 73-75, 81-83, 89-91, 97-99, 105-107, 113-115, 121-123, 129-131, 137-139, 145-147, 153-155, 161-163, 169-171, 177-179, 185-187, 193-195, 201-203, 209-211, 217-219, 225-227, 233-235, 241-243, 249-251, 257-259, 265-267, 273-275, 281-283, 289-291, 297-299, 305-307, 313-315, 321-323, 329-331, 337-339, 345-347, and 353-355. Illustrative examples of heavy chain CDRs that are suitable for constructing anti-ROR1 CARs contemplated in particular embodiments include, but are not limited to the CDR sequences set forth in SEQ ID NOs: 4-6, 12-14, 20-22, 28-30, 36-38, 44-46, 52-54, 60-62, 68-70, 76-78, 84-86, 92-94, 100-102, 108-110, 116-118, 124-126, 132-134, 140-142, 148-150, 156-158, 164-166, 172-174, 180-182, 188-190, 196-198, 204-206, 212-214, 220-222, 228-230, 236-238, 244-246, 252-254, 260-262, 268-270, 276-278, 284-286, 292-294, 300-302, 308-310, 316-318, 324-326, 332-334, 340-342, 348-350, and 356-358.

References to “VL” or “VL” refer to the variable region of an immunoglobulin light chain, including that of an antibody, Fv, scFv, dsFv, Fab, or other antibody fragment as disclosed herein. Illustrative examples of light chain variable regions that are suitable for constructing anti-ROR1 CARs contemplated in particular embodiments include, but are not limited to the light chain variable region sequences set forth in SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, 311, 319, 327, 335, 343, 351, and 359.

References to “VH” or “VH” refer to the variable region of an immunoglobulin heavy chain, including that of an antibody, Fv, scFv, dsFv, Fab, or other antibody fragment as disclosed herein. Illustrative examples of heavy chain variable regions that are suitable for constructing anti-ROR1 CARs contemplated in particular embodiments include, but are not limited to the heavy chain variable region sequences set forth in SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, 312, 320, 328, 336, 344, 352, and 360.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In particular preferred embodiments, a CAR comprises antigen-specific binding domain that is a chimeric antibody or antigen binding fragment thereof.

In preferred embodiments, the antibody is a human antibody (such as a human monoclonal antibody) or fragment thereof that specifically binds to a human ROR1 polypeptide. Human antibodies can be constructed by combining Fv clone variable domain sequence(s) selected from human-derived phage display libraries with known human constant domain sequences(s) as described above. Alternatively, human monoclonal antibodies may be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991). In addition, transgenic animals (e.g., mice) can be used to produce a full repertoire of human antibodies in the absence of endogenous immunoglobulin production. See, e.g., Jakobovits et al., PNAS USA, 90: 2551 (1993); Jakobovits et al., Nature, 362: 255 (1993); Bruggermann et al., Year in Immunol., 7: 33 (1993). Gene shuffling can also be used to derive human antibodies from non-human, e.g., rodent antibodies, where the human antibody has similar affinities and specificities to the starting non-human antibody. See WO 93/06213. Unlike traditional humanization of non-human antibodies by CDR grafting, this technique provides completely human antibodies, which have no FR or CDR residues of non-human origin.

In one embodiment, a CAR comprises a “humanized” antibody. A humanized antibody is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. Humanized or other monoclonal antibodies can have additional conservative amino acid substitutions, which have substantially no effect on antigen binding or other immunoglobulin functions. Humanized antibodies can be constructed by means of genetic engineering (see for example, U.S. Pat. No. 5,585,089).

In particular embodiments, an anti-ROR1 antibody or antigen binding fragment thereof, includes but is not limited to a Camel Ig (a camelid antibody (VHH)), Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, Nanobody).

“Camel Ig” or “camelid VHH” as used herein refers to the smallest known antigen-binding unit of a heavy chain antibody (Koch-Nolte, et al, FASEB J., 21: 3490-3498 (2007)). A “heavy chain antibody” or a “camelid antibody” refers to an antibody that contains two VH domains and no light chains (Riechmann L. et al, J. Immunol. Methods 231:25-38 (1999); WO94/04678; WO94/25591; U.S. Pat. No. 6,005,079).

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three hypervariable regions (HVRs) of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., PNAS USA 90: 6444-6448 (1993). Triabodies and tetrabodies are also described in Hudson et al., Nat. Med. 9:129-134 (2003).

“Single domain antibody” or “sdAb” or “nanobody” refers to an antibody fragment that consists of the variable region of an antibody heavy chain (VH domain) or the variable region of an antibody light chain (VL domain) (Holt, L., et al, Trends in Biotechnology, 21(11): 484-490).

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. For a review of scFv, see, e.g., Pluckthiin, in The Pharmacology of Monoclonal Antibodies, vol. 113, Rosenburg and Moore eds., (Springer-Verlag, New York, 1994), pp. 269-315.

In preferred embodiments, a CAR comprises antigen-specific binding domain that is an scFv and may be a murine, human or humanized scFv. Single chain antibodies may be cloned form the V region genes of a hybridoma specific for a desired target. The production of such hybridomas has become routine. A technique which can be used for cloning the variable region heavy chain (V_{H}) and variable region light chain (V_{L}) has been described, for example, in Orlandi et al., PNAS, 1989; 86: 3833-3837.

In particular embodiments, the antigen-specific binding domain is an scFv that binds a human ROR1 polypeptide.

Illustrative examples of variable light chains that are suitable for constructing anti-ROR1 CARs contemplated in particular embodiments include, but are not limited to the amino acid sequences set forth in SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, 311, 319, 327, 335, 343, 351, and 359.

Illustrative examples of variable heavy chains that are suitable for constructing anti-ROR1 CARs contemplated in particular embodiments include, but are not limited to the amino acid sequences set forth in SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, 312, 320, 328, 336, 344, 352, and 360.

An exemplary ROR1-specific binding domain is an immunoglobulin variable region specific for ROR1 that comprises at least one human framework region. A “human framework region” refers to a wild type (i.e., naturally occurring) framework region of a human immunoglobulin variable region, an altered framework region of a human immunoglobulin variable region with less than about 50% (e.g., preferably less than about 45%, 40%, 30%, 25%, 20%, 15%, 10%, 5%, or 1%) of the amino acids in the region are deleted or substituted (e.g., with one or more amino acid residues of a nonhuman immunoglobulin framework region at corresponding positions), or an altered framework region of a nonhuman immunoglobulin variable region with less than about 50% (e.g., less than 45%, 40%, 30%, 25%, 20%, 15%, 10%, or 5%) of the amino acids in the region deleted or substituted (e.g., at positions of exposed residues and/or with one or more amino acid residues of a human immunoglobulin framework region at corresponding positions) so that, in one aspect, immunogenicity is reduced.

In certain embodiments, a human framework region is a wild type framework region of a human immunoglobulin variable region. In certain other embodiments, a human framework region is an altered framework region of a human immunoglobulin variable region with amino acid deletions or substitutions at one, two, three, four, five, six, seven, eight, nine, ten or more positions. In other embodiments, a human framework region is an altered framework region of a non-human immunoglobulin variable region with amino acid deletions or substitutions at one, two, three, four, five, six, seven, eight, nine, ten or more positions.

In particular embodiments, an ROR1-specific binding domain comprises at least one, two, three, four, five, six, seven or eight human framework regions (FR) selected from human light chain FR1, human heavy chain FR1, human light chain FR2, human heavy chain FR2, human light chain FR3, human heavy chain FR3, human light chain FR4, and human heavy chain FR4.

Human FRs that may be present in an ROR1-specific binding domains also include variants of the exemplary FRs provided herein in which one, two, three, four, five, six, seven, eight, nine, ten or more amino acids of the exemplary FRs have been substituted or deleted.

In certain embodiments, an ROR1-specific binding domain comprises (a) a humanized light chain variable region that comprises a human light chain FR1, a human light chain FR2, a human light chain FR3, and a human light chain FR4, and (b) a humanized heavy chain variable region that comprises a human heavy chain FR1, a human heavy chain FR2, a human heavy chain FR3, and a human heavy chain FR4.

ROR1-specific binding domains provided herein also comprise one, two, three, four, five, or six CDRs. Such CDRs may be nonhuman CDRs or altered nonhuman CDRs selected from CDRL1, CDRL2 and CDRL3 of the light chain and CDRH1, CDRH2 and CDRH3 of the heavy chain. In certain embodiments, an ROR1-specific binding domain comprises (a) a light chain variable region that comprises a light chain CDRL1, a light chain CDRL2, and a light chain CDRL3, and (b) a heavy chain variable region that comprises a heavy chain CDRH1, a heavy chain CDRH2, and a heavy chain CDRH3.

In one embodiment, an ROR1-specific binding domain comprises light chain CDR sequences set forth in SEQ ID NOs: 1-3, 9-11, 17-19, 25-27, 33-35, 41-43, 49-51, 57-59, 65-67, 73-75, 81-83, 89-91, 97-99, 105-107, 113-115, 121-123, 129-131, 137-139, 145-147, 153-155, 161-163, 169-171, 177-179, 185-187, 193-195, 201-203, 209-211, 217-219, 225-227, 233-235, 241-243, 249-251, 257-259, 265-267, 273-275, 281-283, 289-291, 297-299, 305-307, 313-315, 321-323, 329-331, 337-339, 345-347, and 353-355.

In a particular embodiment, an ROR1-specific binding domain comprises light chain CDR sequences with at least 85%, 86%, 87%, 88%, 89%, 90%, 91%, 92%, 93%, 94%, 95%, 96%, 97%, 98%, or 99% amino acid identity to the light chain CDR sequences set forth in SEQ ID NOs: 1-3, 9-11, 17-19, 25-27, 33-35, 41-43, 49-51, 57-59, 65-67, 73-75, 81-83, 89-91, 97-99, 105-107, 113-115, 121-123, 129-131, 137-139, 145-147, 153-155, 161-163, 169-171, 177-179, 185-187, 193-195, 201-203, 209-211, 217-219, 225-227, 233-235, 241-243, 249-251, 257-259, 265-267, 273-275, 281-283, 289-291, 297-299, 305-307, 313-315, 321-323, 329-331, 337-339, 345-347, and 353-355.

In one embodiment, an ROR1-specific binding domain comprises heavy chain CDR sequences set forth in SEQ ID NOs: 4-6, 12-14, 20-22, 28-30, 36-38, 44-46, 52-54, 60-62, 68-70, 76-78, 84-86, 92-94, 100-102, 108-110, 116-118, 124-126, 132-134, 140-142, 148-150, 156-158, 164-166, 172-174, 180-182, 188-190, 196-198, 204-206, 212-214, 220-222, 228-230, 236-238, 244-246, 252-254, 260-262, 268-270, 276-278, 284-286, 292-294, 300-302, 308-310, 316-318, 324-326, 332-334, 340-342, 348-350, and 356-358.

In a particular embodiment, an ROR1-specific binding domain comprises heavy chain CDR sequences with at least 85%, 86%, 87%, 88%, 89%, 90%, 91%, 92%, 93%, 94%, 95%, 96%, 97%, 98%, or 99% amino acid identity to the heavy chain CDR sequences set forth in SEQ ID NOs: 4-6, 12-14, 20-22, 28-30, 36-38, 44-46, 52-54, 60-62, 68-70, 76-78, 84-86, 92-94, 100-102, 108-110, 116-118, 124-126, 132-134, 140-142, 148-150, 156-158, 164-166, 172-174, 180-182, 188-190, 196-198, 204-206, 212-214, 220-222, 228-230, 236-238, 244-246, 252-254, 260-262, 268-270, 276-278, 284-286, 292-294, 300-302, 308-310, 316-318, 324-326, 332-334, 340-342, 348-350, and 356-358.

References to “VL” or “VL” refer to the variable region of an immunoglobulin light chain, including that of an antibody, Fv, scFv, dsFv, Fab, or other antibody fragment as disclosed herein. Illustrative examples of light chain variable regions that are suitable for constructing anti-ROR1 CARs contemplated in particular embodiments include, but are not limited to the light chain variable region sequences set forth in SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, 311, 319, 327, 335, 343, 351, and 359.

References to “VH” or “VH” refer to the variable region of an immunoglobulin heavy chain, including that of an antibody, Fv, scFv, dsFv, Fab, or other antibody fragment as disclosed herein. Illustrative examples of heavy chain variable regions that are suitable for constructing anti-ROR1 CARs contemplated in particular embodiments include, but are not limited to the heavy chain variable region sequences set forth in SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, 312, 320, 328, 336, 344, 352, and 360.

2. Linkers

In certain embodiments, the CARS comprise linker residues between the various domains, e.g., added for appropriate spacing and conformation of the molecule. In particular embodiments the linker is a variable region linking sequence. A “variable region linking sequence,” is an amino acid sequence that connects the V_{H} and V_{L} domains and provides a spacer function compatible with interaction of the two sub-binding domains so that the resulting polypeptide retains a specific binding affinity to the same target molecule as an antibody that comprises the same light and heavy chain variable regions. In particular embodiments, a linker separates one or more heavy or light chain variable domains, hinge domains, transmembrane domains, co-stimulatory domains, and/or primary signaling domains. In particular embodiments, CARS comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G)ₙ; glycine-serine polymers (G₁₋₅S₁₋₅)., where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR in particular embodiments can include linkers that are all or partially flexible, such that the linker can include a flexible linker as well as one or more portions that confer less flexible structure to provide for a desired CAR structure.

Other exemplary linkers include, but are not limited to the following amino acid sequences: GGG; DGGGS (SEQ ID NO: 362); TGEKP (SEQ ID NO: 363) (see, e.g., Liu et al., PNAS 5525-5530 (1997)); GGRR (SEQ ID NO: 364) (Pomerantz et al. 1995, supra); (GGGGS)ₙ wherein=1, 2, 3, 4 or 5 (SEQ ID NO: 365) (Kim et al., PNAS 93, 1156-1160 (1996.); EGKSSGSGSESKVD (SEQ ID NO: 366) (Chaudhary et al., 1990, Proc. Natl. Acad. Sci. U.S.A. 87:1066-1070); KESGSVSSEQLAQFRSLD (SEQ ID NO: 367) (Bird et al., 1988, Science 242:423-426), GGRRGGGS (SEQ ID NO: 368); LRQRDGERP (SEQ ID NO: 369); LRQKDGGGSERP (SEQ ID NO: 370); LRQKd(GGGS)₂ ERP (SEQ ID NO: 371). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. In one embodiment, the linker comprises the following amino acid sequence: GSTSGSGKPGSGEGSTKG (SEQ ID NO: 372) (Cooper et al., Blood, 101(4): 1637-1644 (2003)).

3. Spacer Domain

In particular embodiments, the binding domain of the CAR is followed by one or more “spacer domains,” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In one embodiment, the spacer domain comprises the CH2 and CH3 of IgG1, IgG4, or IgD.

4. Hinge Domain

The binding domain of the CAR is generally followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region.

An “altered hinge region” refers to (a) a naturally occurring hinge region with up to 30% amino acid changes (e.g., up to 25%, 20%, 15%, 10%, or 5% amino acid substitutions or deletions), (b) a portion of a naturally occurring hinge region that is at least 10 amino acids (e.g., at least 12, 13, 14 or 15 amino acids) in length with up to 30% amino acid changes (e.g., up to 25%, 20%, 15%, 10%, or 5% amino acid substitutions or deletions), or (c) a portion of a naturally occurring hinge region that comprises the core hinge region (which may be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a naturally occurring immunoglobulin hinge region may be substituted by one or more other amino acid residues (e.g., one or more serine residues). An altered immunoglobulin hinge region may alternatively or additionally have a proline residue of a wild type immunoglobulin hinge region substituted by another amino acid residue (e.g., a serine residue).

Illustrative hinge domains suitable for use in the CARs described herein include the hinge region derived from the extracellular regions of type 1 membrane proteins such as CD8α, and CD4, which may be wild-type hinge regions from these molecules or may be altered. In another embodiment, the hinge domain comprises a CD8α hinge region.

In one embodiment, the hinge is a PD-1 hinge or CD152 hinge.

5. Transmembrane (TM) Domain

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from (i.e., comprise at least the transmembrane region(s) of the alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1. In a particular embodiment, the TM domain is synthetic and predominantly comprises hydrophobic residues such as leucine and valine.

In one embodiment, the CARs comprise a TM domain derived from, PD1, CD152, or CD8α. In another embodiment, a CAR comprises a TM domain derived from, PD1, CD152, or CD8α and a short oligo- or polypeptide linker, preferably between 1, 2, 3, 4, 5, 6, 7, 8, 9, or 10 amino acids in length that links the TM domain and the intracellular signaling domain of the CAR. A glycine-serine based linker provides a particularly suitable linker.

6. Intracellular Signaling Domain

In particular embodiments, CARs comprise an intracellular signaling domain. An “intracellular signaling domain,” refers to the part of a CAR that participates in transducing the message of effective anti-ROR1 CAR binding to a human ROR1 polypeptide into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of an immune effector cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

It is known that signals generated through the TCR alone are insufficient for full activation of the T cell and that a secondary or co-stimulatory signal is also required. Thus, T cell activation can be said to be mediated by two distinct classes of intracellular signaling domains: primary signaling domains that initiate antigen-dependent primary activation through the TCR (e.g., a TCR/CD3 complex) and co-stimulatory signaling domains that act in an antigen-independent manner to provide a secondary or co-stimulatory signal. In preferred embodiments, a CAR comprises an intracellular signaling domain that comprises one or more “co-stimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs.

Illustrative examples of ITAM containing primary signaling domains that are useful in particular embodiments include those derived from FcRγ, FcRβ, CD3γ, CD3δ, CD3ε, CD3ζ, CD22, CD79a, CD79b, and CD66d. In particular preferred embodiments, a CAR comprises a CD3ζ primary signaling domain and one or more co-stimulatory signaling domains. The intracellular primary signaling and co-stimulatory signaling domains may be linked in any order in tandem to the carboxyl terminus of the transmembrane domain.

In particular embodiments, CARs comprise one or more co-stimulatory signaling domains to enhance the efficacy and expansion of T cells expressing CAR receptors. As used herein, the term, “co-stimulatory signaling domain,” or “co-stimulatory domain”, refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70. In one embodiment, a CAR comprises one or more co-stimulatory signaling domains selected from the group consisting of CD28, CD137, and CD134, and a CD3ζ primary signaling domain.

In another embodiment, a CAR comprises CD28 and CD137 co-stimulatory signaling domains and a CD3ζ primary signaling domain.

In yet another embodiment, a CAR comprises CD28 and CD134 co-stimulatory signaling domains and a CD3ζ primary signaling domain.

In one embodiment, a CAR comprises CD137 and CD134 co-stimulatory signaling domains and a CD3ζ primary signaling domain.

In one embodiment, a CAR comprises a CD137 co-stimulatory signaling domain and a CD3ζ primary signaling domain.

In one embodiment, a CAR comprises a CD134 co-stimulatory signaling domain and a CD3ζ primary signaling domain.

In one embodiment, a CAR comprises a CD28 co-stimulatory signaling domain and a CD3ζ primary signaling domain.

In particular embodiments, CARS comprise an anti-ROR1 antibody or antigen binding fragment thereof that specifically binds to an ROR1 polypeptide expressed on a cancer cell.

In one embodiment, a CAR comprises an anti-ROR1 scFv that binds an ROR1 polypeptide; a transmembrane domain derived from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1; and one or more intracellular co-stimulatory signaling domains from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70; and a primary signaling domain from FcRγ, FcRβ, CD3γ, CD3δ, CD3ε, CD3, CD22, CD79a, CD79b, and CD66d.

In one embodiment, a CAR comprises an anti-ROR1 scFv that binds an ROR1 polypeptide; a hinge domain selected from the group consisting of: IgG1 hinge/CH2/CH3, IgG4 hinge/CH2/CH3, a PD1 hinge, a CD152 hinge, and a CD8α hinge; a transmembrane domain derived from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1; and one or more intracellular co-stimulatory signaling domains from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70; and a primary signaling domain from FcRγ, FcRβ, CD3γ, CD3δ, CD3ε, CD3ζ, CD22, CD79a, CD79b, and CD66d.

In one embodiment, a CAR comprises an anti-ROR1 scFv that binds an ROR1 polypeptide; a hinge domain selected from the group consisting of: IgG1 hinge/CH2/CH3, IgG4 hinge/CH2/CH3, a PD1 hinge, a CD152 hinge, and a CD8α hinge; a transmembrane domain derived from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1; a short oligo- or polypeptide linker, preferably between 1, 2, 3, 4, 5, 6, 7, 8, 9, or 10 amino acids in length that links the TM domain to the intracellular signaling domain of the CAR; and

one or more intracellular co-stimulatory signaling domains from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70; and a primary signaling domain from FcRγ, Fc CD3γ, CD3δ, CD3ε, CD3, CD22, CD79a, CD79b, and CD66d.

In a particular embodiment, a CAR comprises an anti-ROR1 scFv that binds an ROR1 polypeptide; a hinge domain comprising an IgG1 hinge/CH2/CH3 polypeptide and a CD8α polypeptide; a CD8α transmembrane domain comprising a polypeptide linker of about 3 to about 10 amino acids; a CD137 intracellular co-stimulatory signaling domain; and a CD3ζ primary signaling domain.

In a particular embodiment, a CAR comprises an anti-ROR1 scFv that binds an ROR1 polypeptide; a hinge domain comprising a CD8α polypeptide; a CD8α transmembrane domain comprising a polypeptide linker of about 3 to about 10 amino acids; a CD134 intracellular co-stimulatory signaling domain; and a CD3ζ primary signaling domain.

In a particular embodiment, a CAR comprises an anti-ROR1 scFv that binds an ROR1 polypeptide; a hinge domain comprising a CD8α polypeptide; a CD8α transmembrane domain comprising a polypeptide linker of about 3 to about 10 amino acids; a CD28 intracellular co-stimulatory signaling domain; and a CD3ζ primary signaling domain.

In a particular embodiment, a CAR comprises an anti-ROR1 scFv that binds an ROR1 polypeptide; a hinge domain comprising a PD1 hinge, polypeptide; a PD1 or CD152 transmembrane domain comprising a polypeptide linker of about 3 to about 10 amino acids; a CD137 intracellular co-stimulatory signaling domain; and a CD3ζ primary signaling domain.

In a particular embodiment, a CAR comprises an anti-ROR1 scFv that binds an ROR1 polypeptide; a hinge domain comprising a PD1 hinge, polypeptide; a PD1 or CD152 transmembrane domain comprising a polypeptide linker of about 3 to about 10 amino acids; a CD134 intracellular co-stimulatory signaling domain; and a CD3ζ primary signaling domain.

In a particular embodiment, a CAR comprises an anti-ROR1 scFv that binds an ROR1 polypeptide; a hinge domain comprising a PD1 hinge, polypeptide; a PD1 or CD152 transmembrane domain comprising a polypeptide linker of about 3 to about 10 amino acids; a CD28 intracellular co-stimulatory signaling domain; and a CD3ζ primary signaling domain.

In a particular embodiment, a CAR comprises an anti-ROR1 scFv that binds an ROR1 polypeptide; a hinge domain comprising a CD152 hinge, polypeptide; a PD1 or CD152 transmembrane domain comprising a polypeptide linker of about 3 to about 10 amino acids; a CD137 intracellular co-stimulatory signaling domain; and a CD3ζ primary signaling domain.

In a particular embodiment, a CAR comprises an anti-ROR1 scFv that binds an ROR1 polypeptide; a hinge domain comprising a CD152 hinge, polypeptide; a PD1 or CD152 transmembrane domain comprising a polypeptide linker of about 3 to about 10 amino acids; a CD134 intracellular co-stimulatory signaling domain; and a CD3ζ primary signaling domain.

In a particular embodiment, a CAR comprises an anti-ROR1 scFv that binds an ROR1 polypeptide; a hinge domain comprising a CD152 hinge, polypeptide; a PD1 or CD152 transmembrane domain comprising a polypeptide linker of about 3 to about 10 amino acids; a CD28 intracellular co-stimulatory signaling domain; and a CD3ζ primary signaling domain.

Moreover, the design of the CARS contemplated in particular embodiments enable improved expansion, long-term persistence, and cytotoxic properties in T cells expressing the CARs compared to non-modified T cells or T cells modified to express other CARs.

### D. Polypeptides

Various polypeptides are contemplated herein, including, but not limited to, CAR polypeptides and fragments thereof, cells and compositions comprising the same, and vectors that express polypeptides. In preferred embodiments, a polypeptide comprising one or more CARs is provided. In particular embodiments, the CAR is an anti-ROR1 CAR comprising an amino acid sequence as set forth in any one of SEQ ID NOs: 386-397.

“Polypeptide,” “polypeptide fragment,” “peptide” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the CAR polypeptides comprise a signal (or leader) sequence at the N-terminal end of the protein, which co-translationally or post-translationally directs transfer of the protein. Illustrative examples of suitable signal sequences useful in CARs contemplated in particular embodiments include, but are not limited to the IgG1 heavy chain signal polypeptide, a CD8α signal polypeptide, or a human GM-CSF receptor alpha signal polypeptide. Polypeptides can be prepared using any of a variety of well-known recombinant and/or synthetic techniques. Polypeptides contemplated herein, encompass the CARs of the present disclosure, or sequences that have deletions from, additions to, and/or substitutions of one or more amino acid of a CAR contemplated herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of the CARs by introducing one or more substitutions, deletions, additions and/or insertions into a binding domain, hinge, TM domain, co-stimulatory signaling domain or primary signaling domain of a CAR polypeptide. In particular embodiments, polypeptides include polypeptides having at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% amino acid identity to any of the reference sequences contemplated herein, typically where the variant maintains at least one biological activity of the reference sequence.

Polypeptides include “polypeptide fragments.” Polypeptide fragments refer to a polypeptide, which can be monomeric or multimeric that has an amino-terminal deletion, a carboxyl-terminal deletion, and/or an internal deletion or substitution of a naturally-occurring or recombinantly produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments include functional domains, including antigen-binding domains or fragments of antibodies. In the case of an anti-ROR1 antibody, useful fragments include, but are not limited to: a CDR region, a CDR3 region of the heavy or light chain; a variable region of a heavy or light chain; a portion of an antibody chain or variable region including two CDRs; and the like.

The polypeptide may also be fused in-frame or conjugated to a linker or other sequence for ease of synthesis, purification or identification of the polypeptide (e.g., poly-His), or to enhance binding of the polypeptide to a solid support.

As noted above, in particular embodiments, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a polypeptide variant comprises one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 1.

TABLE 1
Amino Acid Codons

| | One | Three | |
|---|---|---|---|
| | letter | letter | |
| Amino Acids | code | code | Codons |
| Alanine | A | Ala | GCA GCC GCG GCU |
| Cysteine | C | Cys | UGC UGU |
| Aspartic acid | D | Asp | GAC GAU |
| Glutamic acid | E | Glu | GAA GAG |
| Phenylalanine | F | Phe | UUC UUU |
| Glycine | G | Gly | GGA GGC GGG GGU |
| Histidine | H | His | CAC CAU |
| Isoleucine | I | Iso | AUA AUC AUU |
| Lysine | K | Lys | AAA AAG |
| Leucine | L | Leu | UUA UUG CUA CUC CUG |
| | | | CUU |
| Methionine | M | Met | AUG |
| Asparagine | N | Asn | AAC AAU |
| Proline | P | Pro | CCA CCC CCG CCU |
| Glutamine | Q | Gln | CAA CAG |
| Arginine | R | Arg | AGA AGG CGA CGC CGG |
| | | | CGU |
| Serine | S | Ser | AGC AGU UCA UCC UCG |
| | | | UCU |
| Threonine | T | Thr | ACA ACC ACG ACU |
| Valine | V | Val | GUA GUC GUG GUU |
| Tryptophan | W | Trp | UGG |
| Tyrosine | Y | Tyr | UAC UAU |

Guidance in determining which amino acid residues can be substituted, inserted, or deleted without abolishing biological activity can be found using computer programs well known in the art, such as DNASTAR, DNA Strider, Geneious, Mac Vector, or Vector NTI software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

In making such changes, the hydropathic index of amino acids may be considered. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In one embodiment, where expression of two or more polypeptides is desired, the polynucleotide sequences encoding them can be separated by and IRES sequence as discussed elsewhere herein. In another embodiment, two or more polypeptides can be expressed as a fusion protein that comprises one or more self-cleaving polypeptide sequences.

Polypeptides contemplated in particular embodiments include fusion polypeptides. In preferred embodiments, fusion polypeptides and polynucleotides encoding fusion polypeptides are provided, e.g., CARs. Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. The polypeptides of the fusion protein can be in any order or a specified order. Fusion polypeptides or fusion proteins can also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as discussed elsewhere herein.

In one embodiment, a fusion partner comprises a sequence that assists in expressing the protein (an expression enhancer) at higher yields than the native recombinant protein. Other fusion partners may be selected so as to increase the solubility of the protein or to enable the protein to be targeted to desired intracellular compartments or to facilitate transport of the fusion protein through the cell membrane.

Fusion polypeptides may further comprise a polypeptide cleavage signal between each of the polypeptide domains described herein. In addition, a polypeptide cleavage site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus Ma proteases (e.g., tobacco etch virus protease), potyvirus HC proteases, potyvirus P1 (P35) proteases, byovirus Ma proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 373), for example, ENLYFQG (SEQ ID NO: 374) and ENLYFQS (SEQ ID NO: 375), wherein X represents any amino acid (cleavage by TEV occurs between Q and G or Q and S).

In a particular embodiment, self-cleaving peptides include those polypeptide sequences obtained from potyvirus and cardiovirus 2A peptides, FMDV (foot-and-mouth disease virus), equine rhinitis A virus, Thosea asigna virus and porcine teschovirus.

In certain embodiments, the self-cleaving polypeptide site comprises a 2A or 2A-like site, sequence or domain (Donnelly et al., 2001. J. Gen. Virol. 82:1027-1041).

TABLE 2
Exemplary 2A sites include the following
sequences:

| | |
|---|---|
| SEQ ID NO: 376 | LLNFDLLKLAGDVESNPGP |
| SEQ ID NO: 377 | TLNFDLLKLAGDVESNPGP |
| SEQ ID NO: 378 | LLKLAGDVESNPGP |
| SEQ ID NO: 379 | NFDLLKLAGDVESNPGP |
| SEQ ID NO: 380 | QLLNFDLLKLAGDVESNPGP |
| SEQ ID NO: 381 | APVKQTLNFDLLKLAGDVESNPGP |
| SEQ ID NO: 382 | VTELLYRMKRAETYCPRPLLAIHPTEARHKQK |
| | IVAPVKQT |
| SEQ ID NO: 383 | LNFDLLKLAGDVESNPGP |
| SEQ ID NO: 384 | LLAIHPTEARHKQKIVAPVKQTLNFDLLKLAG |
| | DVESNPGP |
| SEQ ID NO: 385 | EARHKQKIVAPVKQTLNFDLLKLAGDVESNPG |
| | P |

In preferred embodiments, a polypeptide comprises a CAR polypeptide.

### E. Polynucleotides

In preferred embodiments, a polynucleotide encoding one or more CAR polypeptides is provided. As used herein, the terms “polynucleotide” or “nucleic acid” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. In particular embodiments, polynucleotides include polynucleotides or variants having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% sequence identity to any of the reference sequences contemplated herein. In various illustrative embodiments, polynucleotides include expression vectors, viral vectors, and transfer plasmids, and compositions and cells comprising the same. In various illustrative embodiments, polynucleotides are contemplated that encode a polypeptide, including, but not limited to the polypeptide sequences set forth in SEQ ID NOs: 1-397.

In particular embodiments, polynucleotides are provided that encode at least about 5, 10, 25, 50, 100, 150, 200, 250, 300, 350, 400, 500, 1000, 1250, 1500, 1750, or 2000 or more contiguous amino acid residues of a polypeptide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T AC 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The term “nucleic acid cassette” as used herein refers to genetic sequences within a vector which can express a RNA, and subsequently a protein. The nucleic acid cassette contains the gene of interest, e.g., a CAR. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette contains the sequence of a chimeric antigen receptor used to increase the cytotoxicity of cancer cells that express ROR1. The cassette can be removed and inserted into a plasmid or viral vector as a single unit.

In particular embodiments, polynucleotides include at least one polynucleotide-of-interest. As used herein, the term “polynucleotide-of-interest” refers to a polynucleotide encoding a polypeptide (i.e., a polypeptide-of-interest), inserted into an expression vector that is desired to be expressed. A vector may comprise 1, 2, 3, 4, 5, 6, 7, 8, 9, or 10 polynucleotides-of-interest. In certain embodiments, the polynucleotide-of-interest encodes a polypeptide that provides a therapeutic effect in the treatment or prevention of a disease or disorder. Polynucleotides-of-interest, and polypeptides encoded therefrom, include both polynucleotides that encode wild-type polypeptides, as well as functional variants and fragments thereof. In particular embodiments, a functional variant has at least 80%, at least 90%, at least 95%, or at least 99% identity to a corresponding wild-type reference polynucleotide or polypeptide sequence. In certain embodiments, a functional variant or fragment has at least 50%, at least 60%, at least 70%, at least 80%, or at least 90% of a biological activity of a corresponding wild-type polypeptide.

In one embodiment, the polynucleotide-of-interest does not encode a polypeptide but serves as a template to transcribe miRNA, siRNA, or shRNA, ribozyme, or other inhibitory RNA. In various other embodiments, a polynucleotide comprises a polynucleotide-of-interest encoding a CAR and one or more additional polynucleotides-of-interest including but not limited to an inhibitory nucleic acid sequence including, but not limited to: an siRNA, an miRNA, an snRNA, and a ribozyme.

As used herein, the terms “siRNA” or “short interfering RNA” refer to a short polynucleotide sequence that mediates a process of sequence-specific post-transcriptional gene silencing, translational inhibition, transcriptional inhibition, or epigenetic RNAi in animals (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886). In certain embodiments, an siRNA comprises a first strand and a second strand that have the same number of nucleosides; however, the first and second strands are offset such that the two terminal nucleosides on the first and second strands are not paired with a residue on the complimentary strand. In certain instances, the two nucleosides that are not paired are thymidine resides. The siRNA should include a region of sufficient homology to the target gene, and be of sufficient length in terms of nucleotides, such that the siRNA, or a fragment thereof, can mediate down regulation of the target gene. Thus, an siRNA includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA. Complementarity, or degree of homology with the target strand, is most critical in the antisense strand. While perfect complementarity, particularly in the antisense strand, is often desired, some embodiments include one or more, but preferably 10, 8, 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of the 5′ and/or 3′ terminus. The sense strand need only be sufficiently complementary with the antisense strand to maintain the overall double-strand character of the molecule.

In addition, an siRNA may be modified or include nucleoside analogs. Single stranded regions of an siRNA may be modified or include nucleoside analogs, e.g., the unpaired region or regions of a hairpin structure, e.g., a region which links two complementary regions, can have modifications or nucleoside analogs. Modification to stabilize one or more 3′- or 5′-terminus of an siRNA, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis. Each strand of an siRNA can be equal to or less than 30, 25, 24, 23, 22, 21, or 20 nucleotides in length. The strand is preferably at least 19 nucleotides in length. For example, each strand can be between 21 and 25 nucleotides in length. Preferred siRNAs have a duplex region of 17, 18, 19, 29, 21, 22, 23, 24, or 25 nucleotide pairs, and one or more overhangs of 2-3 nucleotides, preferably one or two 3′ overhangs, of 2-3 nucleotides.

As used herein, the terms “miRNA” or “microRNA” s refer to small non-coding RNAs of 20-22 nucleotides, typically excised from ˜70 nucleotide foldback RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within the 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs encompass both naturally occurring miRNAs as well as artificially designed miRNAs that can specifically target any mRNA sequence. For example, in one embodiment, the skilled artisan can design short hairpin RNA constructs expressed as human miRNA (e.g., miR-30 or miR-21) primary transcripts. This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

As used herein, the terms “shRNA” or “short hairpin RNA” refer to double-stranded structure that is formed by a single self-complementary RNA strand. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Greater than 90% sequence identity, or even 100% sequence identity, between the inhibitory RNA and the portion of the target gene is preferred. In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

As used herein, the term “ribozyme” refers to a catalytically active RNA molecule capable of site-specific cleavage of target mRNA. Several subtypes have been described, e.g., hammerhead and hairpin ribozymes. Ribozyme catalytic activity and stability can be improved by substituting deoxyribonucleotides for ribonucleotides at noncatalytic bases. While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art.

A preferred method of delivery of a polynucleotide-of-interest that comprises an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H1 RNA promoter and the human tRNA-val promoter, or a strong constitutive pol II promoter, as described elsewhere herein.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol.

Polynucleotides can be prepared, manipulated and/or expressed using any of a variety of well-established techniques known and available in the art. In order to express a desired polypeptide, a nucleotide sequence encoding the polypeptide, can be inserted into appropriate vector.

Examples of vectors are plasmid, autonomously replicating sequences, and transposable elements. Additional exemplary vectors include, without limitation, plasmids, phagemids, cosmids, transposons, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, the coding sequences of the chimeric proteins disclosed herein can be ligated into such expression vectors for the expression of the chimeric protein in mammalian cells.

In particular embodiments, the vector is a non-integrating vector, including but not limited to, an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

In particular embodiments, a polynucleotide is introduced into a target or host cell using a transposon vector system. In certain embodiments, the transposon vector system comprises a vector comprising transposable elements and a polynucleotide contemplated herein; and a transposase. In one embodiment, the transposon vector system is a single transposase vector system, see, e.g., International Application No. PCT/US07/18922. Exemplary transposases include, but are not limited to: piggyBac, Sleeping Beauty, Mos1, Tc1/mariner, Tol2, mini-Tol2, Tc3, MuA, Himar I, Frog Prince, and derivatives thereof. The piggyBac transposon and transposase are described, for example, in U.S. Pat. No. 6,962,810, which is incorporated herein by reference in its entirety. The Sleeping Beauty transposon and transposase are described, for example, in Izsvak et al., J. Mol. Biol. 302: 93-102 (2000), which is incorporated herein by reference in its entirety. The Tol2 transposon which was first isolated from the medaka fish Oryzias latipes and belongs to the hAT family of transposons is described in Kawakami et al. (2000). Mini-Tol2 is a variant of Tol2 and is described in Balciunas et al. (2006). The Tol2 and Mini-Tol2 transposons facilitate integration of a transgene into the genome of an organism when co-acting with the Tol2 transposase. The Frog Prince transposon and transposase are described, for example, in Miskey et al., Nucleic Acids Res. 31:6873-6881 (2003).

The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, vectors include, but not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPAS), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter (Challita et al., J Virol. 69(2):748-55 (1995)).

In one embodiment, a vector comprises a MND promoter.

In one embodiment, a vector comprises an EF1a promoter comprising the first intron of the human EF1a gene.

In one embodiment, a vector comprises an EF1a promoter that lacks the first intron of the human EF1a gene.

In a particular embodiment, it may be desirable to express a polynucleotide comprising a CAR from a T cell specific promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site specific DNA recombinase. According to certain embodiments the vector comprises at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The vectors may comprise one or more recombination sites for any of a wide variety of site specific recombinases. It is to be understood that the target site for a site specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

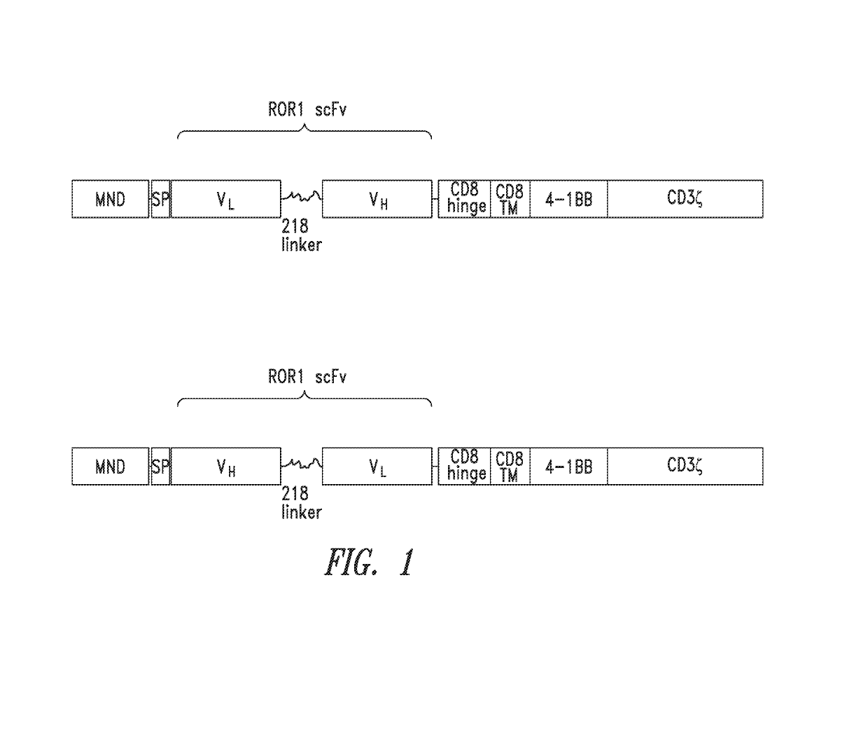

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ, Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO:402), where R is a purine (A or G) (Kozak, 1986. Cell. 44(2):283-92, and Kozak, 1987. Nucleic Acids Res. 15(20):8125-48). In particular embodiments, the vectors comprise polynucleotides that have a consensus Kozak sequence and that encode a desired polypeptide, e.g., a CAR.

In some embodiments, a polynucleotide or cell harboring the polynucleotide utilizes a suicide gene, including an inducible suicide gene to reduce the risk of direct toxicity and/or uncontrolled proliferation. In specific aspects, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

In certain embodiments, vectors comprise gene segments that cause the immune effector cells, e.g., T cells, to be susceptible to negative selection in vivo. By “negative selection” is meant that the infused cell can be eliminated as a result of a change in the in vivo condition of the individual. The negative selectable phenotype may result from the insertion of a gene that confers sensitivity to an administered agent, for example, a compound. Negative selectable genes are known in the art, and include, inter alia the following: the Herpes simplex virus type I thymidine kinase (HSV-I TK) gene (Wigler et al., Cell 11:223, 1977) which confers ganciclovir sensitivity; the cellular hypoxanthine phosphribosyltransferase (HPRT) gene, the cellular adenine phosphoribosyltransferase (APRT) gene, and bacterial cytosine deaminase, (Mullen et al., Proc. Natl. Acad. Sci. USA. 89:33 (1992)).

In some embodiments, genetically modified immune effector cells, such as T cells, comprise a polynucleotide further comprising a positive marker that enables the selection of cells of the negative selectable phenotype in vitro. The positive selectable marker may be a gene which, upon being introduced into the host cell expresses a dominant phenotype permitting positive selection of cells carrying the gene. Genes of this type are known in the art, and include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

Preferably, the positive selectable marker and the negative selectable element are linked such that loss of the negative selectable element necessarily also is accompanied by loss of the positive selectable marker. Even more preferably, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology 11:3374-3378, 1991. In addition, in preferred embodiments, the polynucleotides encoding the chimeric receptors are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra. See also the publications of PCT US91/08442 and PCT/US94/05601, by S. D. Lupton, describing the use of bifunctional selectable fusion genes derived from fusing a dominant positive selectable markers with negative selectable markers.

Preferred positive selectable markers are derived from genes selected from the group consisting of hph, nco, and gpt, and preferred negative selectable markers are derived from genes selected from the group consisting of cytosine deaminase, HSV-I TK, VZV TK, HPRT, APRT and gpt. Especially preferred markers are bifunctional selectable fusion genes wherein the positive selectable marker is derived from hph or neo, and the negative selectable marker is derived from cytosine deaminase or a TK gene or selectable marker.

In various embodiments, the polynucleotide is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, the mRNA encoding a polypeptide is an in vitro transcribed mRNA. As used herein, “in vitro transcribed RNA” refers to RNA, preferably mRNA that has been synthesized in vitro. Generally, the in vitro transcribed RNA is generated from an in vitro transcription vector. The in vitro transcription vector comprises a template that is used to generate the in vitro transcribed RNA.

In particular embodiments, mRNAs may further comprise a comprise a 5′ cap or modified 5′ cap and/or a poly(A) sequence. As used herein, a 5′ cap (also termed an RNA cap, an RNA 7-methylguanosine cap or an RNA m^{7G} cap) is a modified guanine nucleotide that has been added to the “front” or 5′ end of a eukaryotic messenger RNA shortly after the start of transcription. The 5′ cap comprises a terminal group which is linked to the first transcribed nucleotide and recognized by the ribosome and protected from RNases. The capping moiety can be modified to modulate functionality of mRNA such as its stability or efficiency of translation. In a particular embodiment, the mRNA comprises a poly(A) sequence of between about 50 and about 5000 adenines. In one embodiment, the mRNA comprises a poly(A) sequence of between about 100 and about 1000 bases, between about 200 and about 500 bases, or between about 300 and about 400 bases. In one embodiment, the mRNA comprises a poly(A) sequence of about 65 bases, about 100 bases, about 200 bases, about 300 bases, about 400 bases, about 500 bases, about 600 bases, about 700 bases, about 800 bases, about 900 bases, or about 1000 or more bases. poly(A) sequences can be modified chemically or enzymatically to modulate mRNA functionality such as localization, stability or efficiency of translation.

### F. Viral Vectors

In particular embodiments, a cell (e.g., an immune effector cell) is transduced with a retroviral vector, e.g., a lentiviral vector, encoding a CAR. For example, an immune effector cell is transduced with a vector encoding a CAR that comprises a an anti-ROR1 antibody or antigen binding fragment thereof that binds an ROR1 polypeptide, with an intracellular signaling domain of CD3, CD28, 4-1BB, Ox40, or any combinations thereof. Thus, these transduced cells can elicit a CAR-mediated cytotoxic response.

Retroviruses are a common tool for gene delivery (Miller, 2000, Nature. 357: 455-460). In particular embodiments, a retrovirus is used to deliver a polynucleotide encoding a chimeric antigen receptor (CAR) to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), Spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred. In particular embodiments, a lentivirus is used to deliver a polynucleotide comprising a CAR to a cell.

Retroviral vectors and more particularly lentiviral vectors may be used in practicing particular embodiments. Accordingly, the term “retrovirus” or “retroviral vector”, as used herein is meant to include “lentivirus” and “lentiviral vectors” respectively.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

In particular embodiments, the terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles and are present in DNA form in the DNA plasmids.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., 1995. J. of Virology, Vol. 69, No. 4; pp. 2101-2109. Several retroviral vectors use the minimal packaging signal (also referred to as the psi [¹P] sequence) needed for encapsidation of the viral genome. Thus, as used herein, the terms “packaging sequence,” “packaging signal,” “psi” and the symbol “Ψ,” are used in reference to the non-coding sequence required for encapsidation of retroviral RNA strands during viral particle formation.

In various embodiments, vectors comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly A tract. The R region is also defined as being flanked by the U3 and U5 regions. The R region plays a role during reverse transcription in permitting the transfer of nascent DNA from one end of the genome to the other.

As used herein, the term “FLAP element” refers to a nucleic acid whose sequence includes the central polypurine tract and central termination sequences (cPPT and CTS) of a retrovirus, e.g., HIV-1 or HIV-2. In some embodiments, the terms “FLAP element” and “cPPT/FLAP” are used interchangeably to refer to the foregoing FLAP element. Suitable FLAP elements are described in U.S. Pat. No. 6,682,907 and in Zennou, et al., 2000, Cell, 101:173. During HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus. In particular embodiments, the retroviral or lentiviral vector backbones comprise one or more FLAP elements upstream or downstream of the heterologous genes of interest in the vectors. For example, in particular embodiments a transfer plasmid includes a FLAP element. In one embodiment, a vector comprises a FLAP element isolated from HIV-1.

In one embodiment, retroviral or lentiviral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE). Generally, the RNA export element is placed within the 3′ UTR of a gene, and can be inserted as one or multiple copies.

In particular embodiments, expression of heterologous sequences in viral vectors is increased by incorporating post-transcriptional regulatory elements, efficient polyadenylation sites, and optionally, transcription termination signals into the vectors. A variety of post-transcriptional regulatory elements can increase expression of a heterologous nucleic acid at the protein, e.g., woodchuck hepatitis virus post-transcriptional regulatory element (WPRE; Zufferey et al., 1999, J. Virol., 73:2886); the post-transcriptional regulatory element present in hepatitis B virus (HPRE) (Huang et al., Mol. Cell. Biol., 5:3864); and the like (Liu et al., 1995, Genes Dev., 9:1766). In particular embodiments, vectors comprise a post-transcriptional regulatory element such as a WPRE or HPRE.

In particular embodiments, vectors lack or do not comprise a post-transcriptional regulatory element such as a WPRE or HPRE because in some instances these elements increase the risk of cellular transformation and/or do not substantially or significantly increase the amount of mRNA transcript or increase mRNA stability. Therefore, in some embodiments, vectors lack or do not comprise a WPRE or HPRE as an added safety measure.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “poly(A) site” or “poly(A) sequence” as used herein denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Efficient polyadenylation of the recombinant transcript is desirable as transcripts lacking a poly(A) tail are unstable and are rapidly degraded. Illustrative examples of poly(A) signals that can be used in a vector, includes an ideal poly(A) sequence (e.g., AATAAA, ATTAAA, AGTAAA), a bovine growth hormone poly(A) sequence (BGHpA), a rabbit β-globin poly(A) sequence (rβgpA), or another suitable heterologous or endogenous poly(A) sequence known in the art.

In certain embodiments, a retroviral or lentiviral vector further comprises one or more insulator elements. Insulators elements may contribute to protecting lentivirus-expressed sequences, e.g., therapeutic polypeptides, from integration site effects, which may be mediated by cis-acting elements present in genomic DNA and lead to deregulated expression of transferred sequences (i.e., position effect; see, e.g., Burgess-Beusse et al., 2002, Proc. Natl. Acad. Sci., USA, 99:16433; and Zhan et al., 2001, Hum. Genet., 109:471). In some embodiments, transfer vectors comprise one or more insulator element the 3′ LTR and upon integration of the provirus into the host genome, the provirus comprises the one or more insulators at both the 5′ LTR and/or 3′ LTR, by virtue of duplicating the 3′ LTR. Suitable insulators for use in particular embodiments include, but are not limited to, the chicken β-globin insulator (see Chung et al., 1993. Cell 74:505; Chung et al., 1997. PNAS 94:575; and Bell et al., 1999. Cell 98:387, incorporated by reference herein). Examples of insulator elements include, but are not limited to, an insulator from an β-globin locus, such as chicken HS4.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid.

In various embodiments, the vectors comprise a promoter operably linked to a polynucleotide encoding a CAR polypeptide. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase therapeutic gene expression (e.g., poly (A) sequences), and may optionally comprise a WPRE or HPRE.

In a particular embodiment, the transfer vector comprises a left (5′) retroviral LTR; a central polypurine tract/DNA flap (cPPT/FLAP); a retroviral export element; a promoter active in a T cell, operably linked to a polynucleotide encoding CAR polypeptide contemplated herein; and a right (3′) retroviral LTR; and optionally a WPRE or HPRE.

In a particular embodiment, the transfer vector comprises a left (5′) retroviral LTR; a retroviral export element; a promoter active in a T cell, operably linked to a polynucleotide encoding CAR polypeptide contemplated herein; a right (3′) retroviral LTR; and a poly (A) sequence; and optionally a WPRE or HPRE. In another particular embodiment, a lentiviral vector comprises: a left (5′) LTR; a cPPT/FLAP; an RRE; a promoter active in a T cell, operably linked to a polynucleotide encoding CAR polypeptide contemplated herein; a right (3′) LTR; and a polyadenylation sequence; and optionally a WPRE or HPRE.

In a certain embodiment, a lentiviral vector comprises: a left (5′) HIV-1 LTR; a Psi (Ψ) packaging signal; a cPPT/FLAP; an RRE; a promoter active in a T cell, operably linked to a polynucleotide encoding CAR polypeptide contemplated herein; a right (3′) self-inactivating (SIN) HIV-1 LTR; and a rabbit β-globin polyadenylation sequence; and optionally a WPRE or HPRE.

In another embodiment, a vector comprises: at least one LTR; a central polypurine tract/DNA flap (cPPT/FLAP); a retroviral export element; and a promoter active in a T cell, operably linked to a polynucleotide encoding CAR polypeptide contemplated herein; and optionally a WPRE or HPRE.

In particular embodiment, a vector comprises at least one LTR; a cPPT/FLAP; an RRE; a promoter active in a T cell, operably linked to a polynucleotide encoding CAR polypeptide contemplated herein; and a polyadenylation sequence; and optionally a WPRE or HPRE.

In a certain embodiment, a vector comprises at least one SIN HIV-1 LTR; a Psi (Ψ) packaging signal; a cPPT/FLAP; an RRE; a promoter active in a T cell, operably linked to a polynucleotide encoding CAR polypeptide contemplated herein; and a rabbit β-globin polyadenylation sequence; and optionally a WPRE or HPRE.

A “host cell” includes cells electroporated, transfected, infected, or transduced in vivo, ex vivo, or in vitro with a recombinant vector or a polynucleotide. Host cells may include packaging cells, producer cells, and cells infected with viral vectors. In particular embodiments, host cells infected with viral vector are administered to a subject in need of therapy. In certain embodiments, the term “target cell” is used interchangeably with host cell and refers to transfected, infected, or transduced cells of a desired cell type. In preferred embodiments, the target cell is a T cell.

Large scale viral particle production is often necessary to achieve a reasonable viral titer. Viral particles are produced by transfecting a transfer vector into a packaging cell line that comprises viral structural and/or accessory genes, e.g., gag, pol, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. A retroviral/lentiviral transfer vector can be introduced into a packaging cell line, via transfection, transduction or infection, to generate a producer cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120. Preferably, the viral env proteins expressed by packaging cells are encoded on a separate vector from the viral gag and pol genes, as has been previously described.

Illustrative examples of retroviral-derived env genes which can be employed in particular embodiments include, but are not limited to: MLV envelopes, 10A1 envelope, BAEV, FeLV-B, RD114, SSAV, Ebola, Sendai, FPV (Fowl plague virus), and influenza virus envelopes. Similarly, genes encoding envelopes from RNA viruses (e.g., RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples include, but are not limited to, FeLV, VEE, HFVW, WDSV, SFV, Rabies, ALV, BIV, BLV, EBV, CAEV, SNV, ChTLV, STLV, MPMV, SMRV, RAV, FuSV, MH2, AEV, AMV, CT10, and EIAV.

In other embodiments, envelope proteins for pseudotyping a virus include, but are not limited to any of the following virus: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephaliltis causing virus.

In one embodiment, packaging cells are provided, which produce recombinant retrovirus, e.g., lentivirus, pseudotyped with the VSV-G glycoprotein.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells. In a preferred embodiment, lentiviral envelope proteins are pseudotyped with VSV-G. In one embodiment, packaging cells are provided which produce recombinant retrovirus, e.g., lentivirus, pseudotyped with the VSV-G envelope glycoprotein.

As used herein, the term “packaging cell lines” is used in reference to cell lines that do not contain a packaging signal, but do stably or transiently express viral structural proteins and replication enzymes (e.g., gag, pol and env) which are necessary for the correct packaging of viral particles. Any suitable cell line can be employed to prepare packaging cells. Generally, the cells are mammalian cells. In a particular embodiment, the cells used to produce the packaging cell line are human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In preferred embodiments, the packaging cells are 293 cells, 293T cells, or A549 cells. In another preferred embodiment, the cells are A549 cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Methods of preparing viral stock solutions are known in the art and are illustrated by, e.g., Y. Soneoka et al. (1995) NucL Acids Res. 23:628-633, and N. R. Landau et al. (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

The delivery of a gene(s) or other polynucleotide sequence using a retroviral or lentiviral vector by means of viral infection rather than by transfection is referred to as “transduction.” In one embodiment, retroviral vectors are transduced into a cell through infection and provirus integration. In certain embodiments, a target cell, e.g., a T cell, is “transduced” if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

In particular embodiments, host cells transduced with viral vector that expresses one or more polypeptides, are administered to a subject to treat and/or prevent a B cell malignancy. Other methods relating to the use of viral vectors in gene therapy, which may be utilized according to certain embodiments, can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

### G. Genetically Modified Cells

In various embodiments, cells genetically modified to express the CARs contemplated herein, for use in the treatment of cancer are provided. As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a CAR.

In particular embodiments, the CARs contemplated herein are introduced and expressed in immune effector cells so as to redirect their specificity to a target antigen of interest, e.g., an ROR1 polypeptide. An “immune effector cell,” is any cell of the immune system that has one or more effector functions (e.g., cytotoxic cell killing activity, secretion of cytokines, induction of ADCC and/or CDC). The illustrative immune effector cells contemplated in particular embodiments, are T lymphocytes, in particular cytotoxic T cells (CTLs; CD8+ T cells), TILs, and helper T cells (HTLs; CD4+ T cells. In one embodiment, immune effector cells include natural killer (NK) cells. In one embodiment, immune effector cells include natural killer T (NKT) cells.

Immune effector cells can be autologous/autogeneic (“self”) or non-autologous (“non-self,” e.g., allogeneic, syngeneic or xenogeneic).

“Autologous,” as used herein, refers to cells from the same subject.

“Allogeneic,” as used herein, refers to cells of the same species that differ genetically to the cell in comparison.

“Syngeneic,” as used herein, refers to cells of a different subject that are genetically identical to the cell in comparison.

“Xenogeneic,” as used herein, refers to cells of a different species to the cell in comparison. In preferred embodiments, the cells are allogeneic.

Illustrative immune effector cells used with the CARs contemplated in particular embodiments include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺ CD8⁺ T cell, CD4⁻ CD8⁻ T cell, or any other subset of T cells. Other illustrative populations of T cells suitable for use in particular embodiments include naïve T cells and memory T cells.

As would be understood by the skilled person, other cells may also be used as immune effector cells with the CARs as described herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Thus, in particular embodiments, immune effector cell includes progenitors of immune effectors cells such as hematopoietic stem cells (HSCs) contained within the CD34⁺ population of cells derived from cord blood, bone marrow or mobilized peripheral blood which upon administration in a subject differentiate into mature immune effector cells, or which can be induced in vitro to differentiate into mature immune effector cells.

As used herein, immune effector cells genetically engineered to contain ROR1-specific CAR may be referred to as, “ROR1-specific redirected immune effector cells.”

The term, “CD34⁺ cell,” as used herein refers to a cell expressing the CD34 protein on its cell surface. “CD34,” as used herein refers to a cell surface glycoprotein (e.g., sialomucin protein) that often acts as a cell-cell adhesion factor and is involved in T cell entrance into lymph nodes. The CD34⁺ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

Methods for making the immune effector cells which express the CAR contemplated herein are provided in particular embodiments. In one embodiment, the method comprises transfecting or transducing immune effector cells isolated from an individual such that the immune effector cells express one or more CAR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before and/or after being genetically modified (i.e., transduced or transfected to express a CAR contemplated herein).

In particular embodiments, prior to in vitro manipulation or genetic modification of the immune effector cells described herein, the source of cells is obtained from a subject. In particular embodiments, the CAR-modified immune effector cells comprise T cells.

In particular embodiments, PBMCs may be directly genetically modified to express CARs using methods contemplated herein. In certain embodiments, after isolation of PBMC, T lymphocytes are further isolated and in certain embodiments, both cytotoxic and helper T lymphocytes can be sorted into naïve, memory, and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are genetically modified with the chimeric antigen receptors contemplated herein (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7, 175,843; 5,883,223; 6,905,874; 6,797,514; 6,867,041; and U.S. Patent Application Publication No. 20060121005.

In one embodiment, CD34⁺ cells are transduced with a nucleic acid construct contemplated herein. In certain embodiments, the transduced CD34⁺ cells differentiate into mature immune effector cells in vivo following administration into a subject, generally the subject from whom the cells were originally isolated. In another embodiment, CD34⁺ cells may be stimulated in vitro prior to exposure to or after being genetically modified with a CAR as described herein, with one or more of the following cytokines: Flt-3 ligand (FLT3), stem cell factor (SCF), megakaryocyte growth and differentiation factor (TPO), IL-3 and IL-6 according to the methods described previously (Asheuer et al., 2004; Imren, et al., 2004).

In particular embodiments, a population of modified immune effector cells for the treatment of cancer comprises a CAR as disclosed herein. For example, a population of modified immune effector cells are prepared from peripheral blood mononuclear cells (PBMCs) obtained from a patient diagnosed with B cell malignancy described herein (autologous donors). The PBMCs form a heterogeneous population of T lymphocytes that can be CD4⁺, CD8⁺, or CD4⁺ and CD8⁺.

The PBMCs also can include other cytotoxic lymphocytes such as NK cells or NKT cells. An expression vector carrying the coding sequence of a CAR contemplated in particular embodiments is introduced into a population of human donor T cells, NK cells or NKT cells. In particular embodiments, successfully transduced T cells that carry the expression vector can be sorted using flow cytometry to isolate CD3 positive T cells and then further propagated to increase the number of these CAR protein expressing T cells in addition to cell activation using anti-CD3 antibodies and or anti-CD28 antibodies and IL-2 or any other methods known in the art as described elsewhere herein. Standard procedures are used for cryopreservation of T cells expressing the CAR protein T cells for storage and/or preparation for use in a human subject. In one embodiment, the in vitro transduction, culture and/or expansion of T cells are performed in the absence of non-human animal derived products such as fetal calf serum and fetal bovine serum. Since a heterogeneous population of PBMCs is genetically modified, the resultant transduced cells are a heterogeneous population of modified cells comprising an anti-ROR1 CAR as contemplated herein.

In a further embodiment, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells, with a proportion of the modified cells expressing more than one different CAR proteins.

### H. T Cell Manufacturing Methods

In various embodiments, genetically modified T cells are expanded by contact with an agent that stimulates a CD3 TCR complex associated signal and a ligand that stimulates a co-stimulatory molecule on the surface of the T cells.

In particular embodiments, PBMCs or isolated T cells are contacted with a stimulatory agent and costimulatory agent, such as soluble anti-CD3 and anti-CD28 antibodies, or antibodies attached to a bead or other surface, in a culture medium with appropriate cytokines, such as IL-2, IL-7, and/or IL-15.

In particular embodiments, PBMCs or isolated T cells are contacted with a stimulatory agent and costimulatory agent, such as soluble anti-CD3 and anti-CD28 antibodies, or antibodies attached to a bead or other surface, in a culture medium with appropriate cytokines, such as IL-2, IL-7, and/or IL-15 and/or one or more agents that modulate a PI3K/Akt/mTOR cell signaling pathway.

In preferred embodiments, the T cells manufactured by the methods contemplated herein provide improved adoptive immunotherapy compositions. Without wishing to be bound to any particular theory, it is believed that the T cell compositions manufactured by the methods in particular embodiments contemplated herein are imbued with superior properties, including increased survival, expansion in the relative absence of differentiation, and persistence in vivo. In one embodiment, a method of manufacturing T cells comprises contacting the cells with one or more agents that modulate a PI3K cell signaling pathway. In one embodiment, a method of manufacturing T cells comprises contacting the cells with one or more agents that modulate a PI3K/Akt/mTOR cell signaling pathway. In various embodiments, the T cells may be obtained from any source and contacted with the agent during the activation and/or expansion phases of the manufacturing process. The resulting T cell compositions are enriched in developmentally potent T cells that have the ability to proliferate and express one or more of the following biomarkers: CD62L, CCR7, CD28, CD27, CD122, CD127, CD197, CD38, and CD8. In one embodiment, populations of cell comprising T cells, that have been treated with one or more PI3K inhibitors is enriched for a population of CD8⁺ T cells co-expressing one or more or, or all of, the following biomarkers: CD62L, CD127, CD197, and CD38.

In one embodiment, populations of cell comprising T cells, that have been treated with one or more PI3K inhibitors is enriched for a population of CD8⁺ T cells co-expressing one or more or, or all of, the following biomarkers: CD62L, CD127, CD27, and CD8.

In one embodiment, modified T cells comprising maintained levels of proliferation and decreased differentiation are manufactured. In a particular embodiment, T cells are manufactured by stimulating T cells to become activated and to proliferate in the presence of one or more stimulatory signals and an agent that is an inhibitor of a PI3K cell signaling pathway.

The T cells can then be modified to express an anti-ROR1 CARs. In one embodiment, the T cells are modified by transducing the T cells with a viral vector comprising an anti-ROR1 CAR contemplated herein. In a certain embodiment, the T cells are modified prior to stimulation and activation in the presence of an inhibitor of a PI3K cell signaling pathway. In another embodiment, T cells are modified after stimulation and activation in the presence of an inhibitor of a PI3K cell signaling pathway. In a particular embodiment, T cells are modified within 12 hours, 24 hours, 36 hours, or 48 hours of stimulation and activation in the presence of an inhibitor of a PI3K cell signaling pathway.

After T cells are activated, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

In various embodiments, T cell compositions are manufactured in the presence of one or more inhibitors of a PI3K/Akt/mTOR cell signaling pathway. The inhibitors may target one or more activities in the pathway or a single activity. Without wishing to be bound to any particular theory, it is contemplated that treatment or contacting T cells with one or more inhibitors of the PI3K pathway during the stimulation, activation, and/or expansion phases of the manufacturing process preferentially increases young T cells, thereby producing superior therapeutic T cell compositions.

In a particular embodiment, a method for increasing the proliferation of T cells expressing an engineered T cell receptor is provided. Such methods may comprise, for example, harvesting a source of T cells from a subject, stimulating and activating the T cells in the presence of one or more inhibitors of the PI3K pathway, modification of the T cells to express an anti-ROR1 CAR, and expanding the T cells in culture.

In a certain embodiment, a method for producing populations of T cells enriched for expression of one or more of the following biomarkers: CD62L, CCR7, CD28, CD27, CD122, CD127, CD197, CD38, and CD8 is contemplated. In one embodiment, young T cells comprise one or more of, or all of the following biological markers: CD62L, CD127, CD197, and CD38.

In one embodiment, young T cells comprise one or more of, or all of the following biological markers: CD62L, CD127, CD27, and CD8.

In one embodiment, the young T cells lack expression of CD57, CD244, CD160, PD-1, CTLA4, TIM3, and LAG3 are provided. As discussed elsewhere herein, the expression levels young T cell biomarkers is relative to the expression levels of such markers in more differentiated T cells or immune effector cell populations.

In one embodiment, peripheral blood mononuclear cells (PBMCs) are used as the source of T cells in the T cell manufacturing methods contemplated herein. PBMCs form a heterogeneous population of T lymphocytes that can be CD4⁺, CD8⁺, or CD4⁺ and CD8⁺ and can include other mononuclear cells such as monocytes, B cells, NK cells and NKT cells. An expression vector comprising a polynucleotide encoding an engineered TCR or CAR contemplated in particular embodiments are introduced into a population of human donor T cells, NK cells or NKT cells. In a particular embodiment, successfully transduced T cells that carry the expression vector can be sorted using flow cytometry to isolate CD3 positive T cells and then further propagated to increase the number of the modified T cells in addition to cell activation using anti-CD3 antibodies and or anti-CD28 antibodies and IL-2, IL-7, and/or IL-15.

Manufacturing methods contemplated herein may further comprise cryopreservation of modified T cells for storage and/or preparation for use in a human subject. In one embodiment, a method of storing genetically modified murine, human or humanized CAR protein expressing immune effector cells which target an ROR1 expressing cell, comprises cryopreserving the immune effector cells such that the cells remain viable upon thawing. A fraction of the immune effector cells expressing the CAR proteins can be cryopreserved by methods known in the art to provide a permanent source of such cells for the future treatment of patients afflicted with an ROR1 expressing cancer cell. T cells are cryopreserved such that the cells remain viable upon thawing. When needed, the cryopreserved transformed immune effector cells can be thawed, grown and expanded for more such cells. As used herein, “cryopreserving,” refers to the preservation of cells by cooling to sub-zero temperatures, such as (typically) 77 K or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48). The preferred cooling rate is 1° to 3° C./minute. After at least two hours, the T cells have reached a temperature of −80° C. and can be placed directly into liquid nitrogen (−196° C.) for permanent storage such as in a long-term cryogenic storage vessel.

1. T Cells

The manufacture of improved CAR T cell compositions is provided in particular embodiments. T cells used for CAR T cell production may be autologous/autogeneic (“self”) or non-autologous (“non-self,” e.g., allogeneic, syngeneic or xenogeneic). In preferred embodiments, the T cells are obtained from a mammalian subject. In a more preferred embodiment, the T cells are obtained from a primate subject. In the most preferred embodiment, the T cells are obtained from a human subject.

T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL′ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flowthrough centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

In particular embodiments, a population of cells comprising T cells, e.g., PBMCs, is used in the manufacturing methods contemplated herein. In other embodiments, an isolated or purified population of T cells is used in the manufacturing methods contemplated herein. Cells can be isolated from peripheral blood mononuclear cells (PBMCs) by lysing the red blood cells and depleting the monocytes, for example, by centrifugation through a PERCOLL™ gradient. In some embodiments, after isolation of PBMC, both cytotoxic and helper T lymphocytes can be sorted into naïve, memory, and effector T cell subpopulations either before or after activation, expansion, and/or genetic modification.

In particular embodiments, a population of cells comprising T cells, e.g., PBMCs, is used in the manufacturing methods contemplated herein. In other embodiments, an isolated or purified population of T cells is used in the manufacturing methods contemplated herein. Cells can be isolated from peripheral blood mononuclear cells (PBMCs) by lysing the red blood cells and depleting the monocytes, for example, by centrifugation through a PERCOLL™ gradient. In some embodiments, after isolation of PBMC, both cytotoxic and helper T lymphocytes can be sorted into naïve, memory, and effector T cell subpopulations either before or after activation, expansion, and/or genetic modification.

In particular embodiments, the population of immune effector cells is manufactured from PBMC that are genetically modified to express CARs using methods contemplated herein, but that are not subjected to positive or negative selection. In certain embodiments, after isolation of PBMC, T lymphocytes are further isolated and in certain embodiments, both cytotoxic and helper T lymphocytes can be sorted into naïve, memory, and effector T cell subpopulations either before or after genetic modification and/or expansion.

In certain embodiments, specific subpopulation of T cells, expressing one or more of the following markers: CD3, CD4, CD8, CD28, CD45RA, CD45RO, CD62, CD127, and HLA-DR can be further isolated by positive or negative selection techniques. In one embodiment, a specific subpopulation of T cells, expressing one or more of the markers selected from the group consisting of i) CD62L, CCR7, CD28, CD27, CD122, CD127, CD197; ii) CD62L, CD127, CD197, and CD38 or iii) CD62L, CD127, CD27, and CD8, is further isolated by positive or negative selection techniques. In various embodiments, the manufactured T cell compositions do not express or do not substantially express one or more of the following markers: CD57, CD244, CD160, PD-1, CTLA4, TIM3, and LAG3.

In one embodiment, expression of one or more of the markers selected from the group consisting of i) CD62L, CD127, CD197, and CD38 or ii) CD62L, CD127, CD27, and CD8, is increased at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, or more compared to a population of T cells activated and expanded without a PI3K inhibitor. In one embodiment, the T cells comprise CD8⁺ T cells.

In one embodiment, expression of one or more of the markers selected from the group consisting of CD57, CD244, CD160, PD-1, CTLA4, TIM3, and LAG3 is decreased at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, or more compared to a population of T cells activated and expanded with a PI3K inhibitor. In one embodiment, the T cells comprise CD8⁺ T cells.

In one embodiment, the manufacturing methods contemplated herein increase the number CAR T cells comprising one or more markers of naïve or developmentally potent T cells. Without wishing to be bound to any particular theory, the present inventors believe that treating a population of cells comprising T cells with one or more PI3K inhibitors results in an increase an expansion of developmentally potent T cells and provides a more robust and efficacious adoptive CAR T cell immunotherapy compared to existing CAR T cell therapies.

Illustrative examples of markers of naïve or developmentally potent T cells increased in T cells manufactured using the methods contemplated in particular embodiments include, but are not limited to i) CD62L, CD127, CD197, and CD38 or ii) CD62L, CD127, CD27, and CD8. In particular embodiments, naïve T cells do not express do not express or do not substantially express one or more of the following markers: CD57, CD244, CD160, PD-1, BTLA, CD45RA, CTLA4, TIM3, and LAG3.

With respect to T cells, the T cell populations resulting from the various expansion methodologies contemplated herein may have a variety of specific phenotypic properties, depending on the conditions employed. In various embodiments, expanded T cell populations comprise one or more of the following phenotypic markers: CD62L, CD27, CD127, CD197, CD38, CD8, and HLA-DR.

In one embodiment, such phenotypic markers include enhanced expression of one or more of, or all of CD62L, CD127, CD197, and CD38. In particular embodiments, CD8⁺ T lymphocytes characterized by the expression of phenotypic markers of naive T cells including CD62L, CD127, CD197, and CD38 are expanded.

In one embodiment, such phenotypic markers include enhanced expression of one or more of, or all of CD62L, CD127, CD27, and CD8. In particular embodiments, CD8⁺ T lymphocytes characterized by the expression of phenotypic markers of naive T cells including CD62L, CD127, CD27, and CD8 are expanded.

In particular embodiments, T cells characterized by the expression of phenotypic markers of central memory T cells including CD45RO, CD62L, CD127, CD197, and CD38 and negative for granzyme B are expanded. In some embodiments, the central memory T cells are CD45RO⁺, CD62L⁺, CD8⁺ T cells.

In certain embodiments, CD4⁺ T lymphocytes characterized by the expression of phenotypic markers of naïve CD4⁺ cells including CD62L and negative for expression of CD45RA and/or CD45RO are expanded. In some embodiments, CD4⁺ cells characterized by the expression of phenotypic markers of central memory CD4⁺ cells including CD62L and CD45RO positive. In some embodiments, effector CD4⁺ cells are CD62L positive and CD45RO negative.

In certain embodiments, the T cells are isolated from an individual and activated and stimulated to proliferate in vitro prior to being genetically modified to express an anti-ROR1 CAR. In this regard, the T cells may be cultured before and/or after being genetically modified (i.e., transduced or transfected to express an anti-ROR1 CAR contemplated herein).

2. Activation and Expansion

In order to achieve sufficient therapeutic doses of T cell compositions, T cells are often subject to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety. T cells modified to express an anti-ROR1 CAR can be activated and expanded before and/or after the T cells are modified. In addition, T cells may be contacted with one or more agents that modulate a PI3K/Akt/mTOR cell signaling pathway before, during, and/or after activation and/or expansion. In one embodiment, T cells manufactured by the methods contemplated herein undergo one, two, three, four, or five or more rounds of activation and expansion, each of which may include one or more agents that modulate a PI3K/Akt/mTOR cell signaling pathway.

Artificial antigen presenting cells (aAPCs) support ex vivo growth and long-term expansion of functional human CD8⁺ T cells without requiring the addition of exogenous cytokines, in contrast to the use of natural APCs. In particular embodiments, PBMCs or isolated T cells are contacted with a stimulatory agent and costimulatory agent, such as anti-CD3 and anti-CD28 antibodies, generally attached to a bead or other surface, in a culture medium with appropriate cytokines, such as IL-2, IL-7, and/or IL-15.

In other embodiments, artificial APC (aAPC) made by engineering K562, U937, 721.221, T2, and C1R cells to direct the stable expression and secretion, of a variety of costimulatory molecules and cytokines. In a particular embodiment, K32 or U32 aAPCs are used to direct the display of one or more antibody-based stimulatory molecules on the AAPC cell surface. Populations of T cells can be expanded by aAPCs expressing a variety of costimulatory molecules including, but not limited to, CD137L (4-1BBL), CD134L (OX40L), and/or CD80 or CD86. The aAPCs provide an efficient platform to expand genetically modified T cells and to maintain CD28 expression on CD8⁺ T cells. aAPCs provided in WO 03/057171 and US2003/0147869 are hereby incorporated by reference in their entirety.

In one embodiment, a costimulatory ligand is presented on an antigen presenting cell (e.g., an aAPC, dendritic cell, B cell, and the like) that specifically binds a cognate costimulatory molecule on a T cell, thereby providing a signal which, in addition to the primary signal provided by, for instance, binding of a TCR/CD3 complex, mediates a desired T cell response. Suitable costimulatory ligands include, but are not limited to, CD7, B7-1 (CD80), B7-2 (CD86), 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, ILT3, ILT4, an agonist or antibody that binds Tol1 ligand receptor, and a ligand that specifically binds with B7-H3.

In a particular embodiment, a costimulatory ligand comprises an antibody or antigen binding fragment thereof that specifically binds to a costimulatory molecule present on a T cell, including but not limited to, CD27, CD28, 4-1BB, OX40, CD30, CD40, ICOS, lymphocyte function-associated antigen-1 (LFA-1), CD7, LIGHT, NKG2C, B7-H3, and a ligand that specifically binds with CD83.

Suitable costimulatory ligands further include target antigens, which may be provided in soluble form or expressed on APCs or aAPCs that bind engineered TCRs or CARs expressed on modified T cells.

In various embodiments, a method for manufacturing T cells contemplated herein comprises activating a population of cells comprising T cells and expanding the population of T cells. T cell activation can be accomplished by providing a primary stimulation signal through the T cell TCR/CD3 complex or via stimulation of the CD2 surface protein and by providing a secondary costimulation signal through an accessory molecule, e.g., CD28.

The TCR/CD3 complex may be stimulated by contacting the T cell with a suitable CD3 binding agent, e.g., a CD3 ligand or an anti-CD3 monoclonal antibody. Illustrative examples of CD3 antibodies include, but are not limited to, OKT3, G19-4, BC3, and 64.1.

In another embodiment, a CD2 binding agent may be used to provide a primary stimulation signal to the T cells. Illustrative examples of CD2 binding agents include, but are not limited to, CD2 ligands and anti-CD2 antibodies, e.g., the T11.3 antibody in combination with the T11.1 or T11.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986)1 Immunol. 137:1097-1100). Other antibodies which bind to the same epitopes as any of the above described antibodies can also be used. Additional antibodies, or combinations of antibodies, can be prepared and identified by standard techniques as disclosed elsewhere herein.

In addition to the primary stimulation signal provided through the TCR/CD3 complex, or via CD2, induction of T cell responses requires a second, costimulatory signal. In particular embodiments, a CD28 binding agent can be used to provide a costimulatory signal. Illustrative examples of CD28 binding agents include but are not limited to: natural CD 28 ligands, e.g., a natural ligand for CD28 (e.g., a member of the B7 family of proteins, such as B7-1(CD80) and B7-2 (CD86); and anti-CD28 monoclonal antibody or fragment thereof capable of crosslinking the CD28 molecule, e.g., monoclonal antibodies 9.3, B-T3, XR-CD28, KOLT-2, 15E8, 248.23.2, and EX5.3D10.

In one embodiment, the molecule providing the primary stimulation signal, for example a molecule which provides stimulation through the TCR/CD3 complex or CD2, and the costimulatory molecule are coupled to the same surface.

In certain embodiments, binding agents that provide stimulatory and costimulatory signals are localized on the surface of a cell. This can be accomplished by transfecting or transducing a cell with a nucleic acid encoding the binding agent in a form suitable for its expression on the cell surface or alternatively by coupling a binding agent to the cell surface.

In another embodiment, the molecule providing the primary stimulation signal, for example a molecule which provides stimulation through the TCR/CD3 complex or CD2, and the costimulatory molecule are displayed on antigen presenting cells.

In one embodiment, the molecule providing the primary stimulation signal, for example a molecule which provides stimulation through the TCR/CD3 complex or CD2, and the costimulatory molecule are provided on separate surfaces.

In a certain embodiment, one of the binding agents that provide stimulatory and costimulatory signals is soluble (provided in solution) and the other agent(s) is provided on one or more surfaces.

In a particular embodiment, the binding agents that provide stimulatory and costimulatory signals are both provided in a soluble form (provided in solution).

In various embodiments, the methods for manufacturing T cells contemplated herein comprise activating T cells with anti-CD3 and anti-CD28 antibodies.

T cell compositions manufactured by the methods contemplated in particular embodiments comprise T cells activated and/or expanded in the presence of one or more agents that inhibit a PI3K cell signaling pathway. T cells modified to express an anti-ROR1 CAR can be activated and expanded before and/or after the T cells are modified. In particular embodiments, a population of T cells is activated, modified to express an anti-ROR1 CAR, and then cultured for expansion.

In one embodiment, T cells manufactured by the methods contemplated herein comprise an increased number of T cells expressing markers indicative of high proliferative potential and the ability to self-renew but that do not express or express substantially undetectable markers of T cell differentiation. These T cells may be repeatedly activated and expanded in a robust fashion and thereby provide an improved therapeutic T cell composition.

In one embodiment, a population of T cells activated and expanded in the presence of one or more agents that inhibit a PI3K cell signaling pathway is expanded at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, at least 50 fold, at least 100 fold, at least 250 fold, at least 500 fold, at least 1000 fold, or more compared to a population of T cells activated and expanded without a PI3K inhibitor.

In one embodiment, a population of T cells characterized by the expression of markers young T cells are activated and expanded in the presence of one or more agents that inhibit a PI3K cell signaling pathway is expanded at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, at least 50 fold, at least 100 fold, at least 250 fold, at least 500 fold, at least 1000 fold, or more compared the population of T cells activated and expanded without a PI3K inhibitor.

In one embodiment, expanding T cells activated by the methods contemplated herein further comprises culturing a population of cells comprising T cells for several hours (about 3 hours) to about 7 days to about 28 days or any hourly integer value in between. In another embodiment, the T cell composition may be cultured for 14 days. In a particular embodiment, T cells are cultured for about 21 days. In another embodiment, the T cell compositions are cultured for about 2-3 days. Several cycles of stimulation/activation/expansion may also be desired such that culture time of T cells can be 60 days or more.

In particular embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan.

Further illustrative examples of cell culture media include, but are not limited to RPMI 1640, Clicks, AIM-V, DMEM, MEM, a-MEM, F-12, X-Vivo 1 5, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

Illustrative examples of other additives for T cell expansion include, but are not limited to, surfactant, piasmanate, pH buffers such as HEPES, and reducing agents such as N-acetyl-cysteine and 2-mercaptoethanol

Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

3. Agents

In various embodiments, a method for manufacturing T cells is provided that expands undifferentiated or developmentally potent T cells comprising contacting T cells with an agent that modulates a PI3K pathway in the cells. In various embodiments, a method for manufacturing T cells is provided that expands undifferentiated or developmentally potent T cells comprising contacting T cells with an agent that modulates a PI3K/AKT/mTOR pathway in the cells. The cells may be contacted prior to, during, and/or after activation and expansion. The T cell compositions retain sufficient T cell potency such that they may undergo multiple rounds of expansion without a substantial increase in differentiation.

As used herein, the terms “modulate,” “modulator,” or “modulatory agent” or comparable term refer to an agent's ability to elicit a change in a cell signaling pathway. A modulator may increase or decrease an amount, activity of a pathway component or increase or decrease a desired effect or output of a cell signaling pathway. In one embodiment, the modulator is an inhibitor. In another embodiment, the modulator is an activator.

An “agent” refers to a compound, small molecule, e.g., small organic molecule, nucleic acid, polypeptide, or a fragment, isoform, variant, analog, or derivative thereof used in the modulation of a PI3K/AKT/mTOR pathway.

A “small molecule” refers to a composition that has a molecular weight of less than about 5 kD, less than about 4 kD, less than about 3 kD, less than about 2 kD, less than about 1 kD, or less than about 0.5 kD. Small molecules may comprise nucleic acids, peptides, polypeptides, peptidomimetics, peptoids, carbohydrates, lipids, components thereof or other organic or inorganic molecules. Libraries of chemical and/or biological mixtures, such as fungal, bacterial, or algal extracts, are known in the art and can be screened with any of the assays. Examples of methods for the synthesis of molecular libraries can be found in: (Carell et al., 1994a; Carell et al., 1994b; Cho et al., 1993; DeWitt et al., 1993; Gallop et al., 1994; Zuckermann et al., 1994).

An “analog” refers to a small organic compound, a nucleotide, a protein, or a polypeptide that possesses similar or identical activity or function(s) as the compound, nucleotide, protein or polypeptide or compound having the desired activity, but need not necessarily comprise a sequence or structure that is similar or identical to the sequence or structure of the preferred embodiment.

A “derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

In various embodiments, the agent that modulates a PI3K pathway activates a component of the pathway. An “activator,” or “agonist” refers to an agent that promotes, increases, or induces one or more activities of a molecule in a PI3K/AKT/mTOR pathway including, without limitation, a molecule that activates one or more activities of a PI3K.

In various embodiments, the agent that modulates a PI3K pathway inhibits a component of the pathway. An “inhibitor” or “antagonist” refers to an agent that inhibits, decreases, or reduces one or more activities of a molecule in a PI3K/AKT/mTOR pathway including, without limitation, a molecule than inhibits one or more activities of a PI3K. In one embodiment, the inhibitor is a dual molecule inhibitor. In particular embodiment, the inhibitor may inhibit a class of molecules have the same or substantially similar activities (a pan-inhibitor) or may specifically inhibit a molecule's activity (a selective or specific inhibitor). Inhibition may also be irreversible or reversible.

In one embodiment, the inhibitor has an IC50 of at least 1 nM, at least 2 nM, at least 5 nM, at least 10 nM, at least 50 nM, at least 100 nM, at least 200 nM, at least 500 nM, at least 1 μM, at least 10 μM, at least 50 μM, or at least 100 μM. IC50 determinations can be accomplished using any conventional techniques known in the art. For example, an IC50 can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the “IC50” value. Analogously, other inhibitory concentrations can be defined through appropriate determinations of activity.

In various embodiments, T cells are contacted or treated or cultured with one or more modulators of a PI3K/AKT/mTOR pathway at a concentration of at least 1 nM, at least 2 nM, at least 5 nM, at least 10 nM, at least 50 nM, at least 100 nM, at least 200 nM, at least 500 nM, at least 1 μM, at least 10 μM, at least 50 μM, at least 100 μM, or at least 1 M.

In particular embodiments, T cells may be contacted or treated or cultured with one or more modulators of a PI3K/AKT/mTOR pathway for at least 12 hours, 18 hours, at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

The phosphatidyl-inositol-3 kinase/Akt/mammalian target of rapamycin pathway serves as a conduit to integrate growth factor signaling with cellular proliferation, differentiation, metabolism, and survival. PI3Ks are a family of highly conserved intracellular lipid kinases. Class IA PI3Ks are activated by growth factor receptor tyrosine kinases (RTKs), either directly or through interaction with the insulin receptor substrate family of adaptor molecules. This activity results in the production of phosphatidyl-inositol-3,4,5-trisphospate (PIP3) a regulator of the serine/threonine kinase Akt. mTOR acts through the canonical PI3K pathway via 2 distinct complexes, each characterized by different binding partners that confer distinct activities. mTORC1 (mTOR in complex with PRAS40, raptor, and mLST8/GbL) acts as a downstream effector of PI3K/Akt signaling, linking growth factor signals with protein translation, cell growth, proliferation, and survival. mTORC2 (mTOR in complex with rictor, mSIN1, protor, and mLST8) acts as an upstream activator of Akt.

Upon growth factor receptor-mediated activation of PI3K, Akt is recruited to the membrane through the interaction of its pleckstrin homology domain with PIP3, thus exposing its activation loop and enabling phosphorylation at threonine 308 (Thr308) by the constitutively active phosphoinositide-dependent protein kinase 1 (PDK1). For maximal activation, Akt is also phosphorylated by mTORC2, at serine 473 (Ser473) of its C-terminal hydrophobic motif. DNA-PK and HSP have also been shown to be important in the regulation of Akt activity. Akt activates mTORC1 through inhibitory phosphorylation of TSC2, which along with TSC1, negatively regulates mTORC1 by inhibiting the Rheb GTPase, a positive regulator of mTORC1. mTORC1 has 2 well-defined substrates, p70S6K (referred to hereafter as S6K1) and 4E-BP1, both of which critically regulate protein synthesis. Thus, mTORC1 is an important downstream effector of PI3K, linking growth factor signaling with protein translation and cellular proliferation.

a. PI3K Inhibitors

As used herein, the term “PI3K inhibitor” refers to a nucleic acid, peptide, compound, or small organic molecule that binds to and inhibits at least one activity of PI3K. The PI3K proteins can be divided into three classes, class 1 PI3Ks, class 2 PI3Ks, and class 3 PI3Ks. Class 1 PI3Ks exist as heterodimers consisting of one of four p110 catalytic subunits (p110α, p110β, p110δ, and p110γ) and one of two families of regulatory subunits. A PI3K inhibitor preferably targets the class 1 PI3K inhibitors. In one embodiment, a PI3K inhibitor will display selectivity for one or more isoforms of the class 1 PI3K inhibitors (i.e., selectivity for p110α, p110β, p110δ, and p110γ or one or more of p110α, p110β, p110δ, and p110γ). In another aspect, a PI3K inhibitor will not display isoform selectivity and be considered a “pan-PI3K inhibitor.” In one embodiment, a PI3K inhibitor will compete for binding with ATP to the PI3K catalytic domain.

In certain embodiments, a PI3K inhibitor can, for example, target PI3K as well as additional proteins in the PI3K-AKT-mTOR pathway. In particular embodiments, a PI3K inhibitor that targets both mTOR and PI3K can be referred to as either an mTOR inhibitor or a PI3K inhibitor. A PI3K inhibitor that only targets PI3K can be referred to as a selective PI3K inhibitor. In one embodiment, a selective PI3K inhibitor can be understood to refer to an agent that exhibits a 50% inhibitory concentration with respect to PI3K that is at least 10-fold, at least 20-fold, at least 30-fold, at least 50-fold, at least 100-fold, at least 1000-fold, or more, lower than the inhibitor's IC50 with respect to mTOR and/or other proteins in the pathway.

In a particular embodiment, exemplary PI3K inhibitors inhibit PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one embodiment, a PI3K inhibitor inhibits PI3K with an IC50 from about 2 nM to about 100 nm, more preferably from about 2 nM to about 50 nM, even more preferably from about 2 nM to about 15 nM.

Illustrative examples of PI3K inhibitors suitable for use in the T cell manufacturing methods contemplated in particular embodiments include, but are not limited to, BKM120 (class 1 PI3K inhibitor, Novartis), XL147 (class 1 PI3K inhibitor, Exelixis), (pan-PI3K inhibitor, GlaxoSmithKline), and PX-866 (class 1 PI3K inhibitor; p110α, p110β, and p110γ isoforms, Oncothyreon).

Other illustrative examples of selective PI3K inhibitors include, but are not limited to BYL719, GSK2636771, TGX-221, AS25242, CAL-101, ZSTK474, and IPI-145.

Further illustrative examples of pan-PI3K inhibitors include, but are not limited to BEZ235, LY294002, GSK1059615, TG100713, and GDC-0941.

In a preferred embodiment, the PI3K inhibitor is ZSTK474.

b. AKT Inhibitors

As used herein, the term “AKT inhibitor” refers to a nucleic acid, peptide, compound, or small organic molecule that inhibits at least one activity of AKT. AKT inhibitors can be grouped into several classes, including lipid-based inhibitors (e.g., inhibitors that target the pleckstrin homology domain of AKT which prevents AKT from localizing to plasma membranes), ATP-competitive inhibitors, and allosteric inhibitors. In one embodiment, AKT inhibitors act by binding to the AKT catalytic site. In a particular embodiment, Akt inhibitors act by inhibiting phosphorylation of downstream AKT targets such as mTOR In another embodiment, AKT activity is inhibited by inhibiting the input signals to activate Akt by inhibiting, for example, DNA-PK activation of AKT, PDK-1 activation of AKT, and/or mTORC2 activation of Akt.

AKT inhibitors can target all three AKT isoforms, AKT1, AKT2, AKT3 or may be isoform selective and target only one or two of the AKT isoforms. In one embodiment, an AKT inhibitor can target AKT as well as additional proteins in the PI3K-AKT-mTOR pathway. An AKT inhibitor that only targets AKT can be referred to as a selective AKT inhibitor. In one embodiment, a selective AKT inhibitor can be understood to refer to an agent that exhibits a 50% inhibitory concentration with respect to AKT that is at least 10-fold, at least 20-fold, at least 30-fold, at least 50-fold, at least 100-fold, at least 1000-fold, or more lower than the inhibitor's IC50 with respect to other proteins in the pathway.

In a particular embodiment, exemplary AKT inhibitors inhibit AKT with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one embodiment, an AKT inhibits AKT with an IC50 from about 2 nM to about 100 nm, more preferably from about 2 nM to about 50 nM, even more preferably from about 2 nM to about 15 nM.

Illustrative examples of AKT inhibitors for use in combination with auristatin based antibody-drug conjugates include, for example, perifosine (Keryx), MK2206 (Merck), VQD-002 (VioQuest), XL418 (Exelixis), GSK690693, GDC-0068, and PX316 (PROLX Pharmaceuticals).

An illustrative, non-limiting example of a selective Akt1 inhibitor is A-674563.

An illustrative, non-limiting example of a selective Akt2 inhibitor is CCT128930.

In particular embodiments, the Akt inhibitor DNA-PK activation of Akt, PDK-1 activation of Akt, mTORC2 activation of Akt, or HSP activation of Akt.

Illustrative examples of DNA-PK inhibitors include, but are not limited to, NU7441, PI-103, NU7026, PIK-75, and PP-121.

c. mTOR Inhibitors

The terms “mTOR inhibitor” or “agent that inhibits mTOR” refers to a nucleic acid, peptide, compound, or small organic molecule that inhibits at least one activity of an mTOR protein, such as, for example, the serine/threonine protein kinase activity on at least one of its substrates (e.g., p70S6 kinase 1, 4E-BP1, AKT/PKB and eEF2). mTOR inhibitors are able to bind directly to and inhibit mTORC1, mTORC2 or both mTORC1 and mTORC2.

Inhibition of mTORC1 and/or mTORC2 activity can be determined by a reduction in signal transduction of the PI3K/Akt/mTOR pathway. A wide variety of readouts can be utilized to establish a reduction of the output of such signaling pathway. Some non-limiting exemplary readouts include (1) a decrease in phosphorylation of Akt at residues, including but not limited to 5473 and T308; (2) a decrease in activation of Akt as evidenced, for example, by a reduction of phosphorylation of Akt substrates including but not limited to Fox01/O3a T24/32, GSK3a/β; S21/9, and TSC2 T1462; (3) a decrease in phosphorylation of signaling molecules downstream of mTOR, including but not limited to ribosomal S6 S240/244, 70S6K T389, and 4EBP1 T37/46; and (4) inhibition of proliferation of cancerous cells.

In one embodiment, the mTOR inhibitors are active site inhibitors. These are mTOR inhibitors that bind to the ATP binding site (also referred to as ATP binding pocket) of mTOR and inhibit the catalytic activity of both mTORC1 and mTORC2. One class of active site inhibitors suitable for use in the T cell manufacturing methods contemplated in particular embodiments are dual specificity inhibitors that target and directly inhibit both PI3K and mTOR. Dual specificity inhibitors bind to both the ATP binding site of mTOR and PI3K. Illustrative examples of such inhibitors include, but are not limited to: imidazoquinazolines, wortmannin, LY294002, PI-103 (Cayman Chemical), SF1126 (Semafore), BGT226 (Novartis), XL765 (Exelixis) and NVP-BEZ235 (Novartis).

Another class of mTOR active site inhibitors suitable for use in the methods contemplated in particular embodiments selectively inhibit mTORC1 and mTORC2 activity relative to one or more type I phophatidylinositol 3-kinases, e.g., PI3 kinase α, β, γ, or δ. These active site inhibitors bind to the active site of mTOR but not PI3K. Illustrative examples of such inhibitors include, but are not limited to: pyrazolopyrimidines, Torin1 (Guertin and Sabatini), PP242 (2-(4-Amino-1-isopropyl-1H-pyrazolo[3,4-d]pyrimidin-3-yl)-1H-indol-5-ol), PP30, Ku-0063794, WAY-600 (Wyeth), WAY-687 (Wyeth), WAY-354 (Wyeth), and AZD8055 (Liu et al., Nature Review, 8, 627-644, 2009).

In one embodiment, a selective mTOR inhibitor refers to an agent that exhibits a 50% inhibitory concentration (IC50) with respect to mTORC1 and/or mTORC2, that is at least 10-fold, at least 20-fold, at least 50-fold, at least 100-fold, at least 1000-fold, or more, lower than the inhibitor's IC50 with respect to one, two, three, or more type I PI3-kinases or to all of the type I PI3-kinases.

Another class of mTOR inhibitors are referred to herein as “rapalogs”. As used herein the term “rapalogs” refers to compounds that specifically bind to the mTOR FRB domain (FKBP rapamycin binding domain), are structurally related to rapamycin, and retain the mTOR inhibiting properties. The term rapalogs excludes rapamycin. Rapalogs include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Illustrative examples of rapalogs suitable for use in the methods contemplated in particular embodiments include, without limitation, temsirolimus (CC1779), everolimus (RAD001), deforolimus (AP23573), AZD8055 (AstraZeneca), and OSI-027 (OSI).

In one embodiment, the agent is the mTOR inhibitor rapamycin (sirolimus).

In a particular embodiment, exemplary mTOR inhibitors inhibit either mTORC1, mTORC2 or both mTORC1 and mTORC2 with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one aspect, a mTOR inhibitor inhibits either mTORC1, mTORC2 or both mTORC1 and mTORC2 with an IC50 from about 2 nM to about 100 nm, more preferably from about 2 nM to about 50 nM, even more preferably from about 2 nM to about 15 nM.

In one embodiment, exemplary mTOR inhibitors inhibit either PI3K and mTORC1 or mTORC2 or both mTORC1 and mTORC2 and PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one aspect, a mTOR inhibitor inhibits PI3K and mTORC1 or mTORC2 or both mTORC1 and mTORC2 and PI3K with an IC50 from about 2 nM to about 100 nm, more preferably from about 2 nM to about 50 nM, even more preferably from about 2 nM to about 15 nM.

Further illustrative examples of mTOR inhibitors suitable for use in particular embodiments include, but are not limited to AZD8055, INK128, rapamycin, PF-04691502, and everolimus.

mTOR has been shown to demonstrate a robust and specific catalytic activity toward the physiological substrate proteins, p70 S6 ribosomal protein kinase I (p70S6K1) and eIF4E binding protein 1 (4EBP1) as measured by phosphor-specific antibodies in Western blotting.

In one embodiment, the inhibitor of the PI3K/AKT/mTOR pathway is a s6 kinase inhibitor selected from the group consisting of: BI-D1870, H89, PF-4708671, FMK, and AT7867.

### I. Compositions and Formulations

The compositions contemplated herein may comprise one or more polypeptides, polynucleotides, vectors comprising same, genetically modified immune effector cells, etc., as contemplated herein. Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions comprise an amount of CAR-expressing immune effector cells contemplated herein. As used herein, the term “amount” refers to “an amount effective” or “an effective amount” of a genetically modified therapeutic cell, e.g., T cell, to achieve a beneficial or desired prophylactic or therapeutic result, including clinical results.

A “prophylactically effective amount” refers to an amount of a genetically modified therapeutic cell effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mLs or less, even 250 mLs or 100 mLs or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹ or 10¹² cells. In some aspects, particularly since all the infused cells will be redirected to a particular target antigen, lower numbers of cells, in the range of 10⁶/kilogram (10⁶-10¹¹ per patient) may be administered. CAR expressing cell compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIP1α, etc.) as described herein to enhance induction of the immune response.

Generally, compositions comprising the cells activated and expanded as described herein may be utilized in the treatment and prevention of diseases that arise in individuals who are immunocompromised. In particular embodiments, compositions comprising the CAR-modified T cells contemplated herein are used in the treatment of cancer. The CAR-modified T cells may be administered either alone, or as a pharmaceutical composition in combination with carriers, diluents, excipients, and/or with other components such as IL-2 or other cytokines or cell populations. In particular embodiments, pharmaceutical compositions comprise an amount of genetically modified T cells, in combination with one or more pharmaceutically or physiologically acceptable carriers, diluents or excipients.

Pharmaceutical compositions comprising a CAR-expressing immune effector cell population, such as T cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the T cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In one preferred embodiment, compositions comprising T cells contemplated herein are formulated in a solution comprising PlasmaLyte A.

In another preferred embodiment, compositions comprising T cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising cells contemplated herein are formulated in a solution comprising 50:50 PlasmaLyte A to CryoStor CS10.

In a particular embodiment, compositions comprise an effective amount of CAR-expressing immune effector cells, alone or in combination with one or more therapeutic agents. Thus, the CAR-expressing immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated in particular embodiments include cytokines, growth factors, steroids, NSAIDs, DMARDs, anti-inflammatories, chemotherapeutics, radiotherapeutics, therapeutic antibodies, or other active and ancillary agents.

In certain embodiments, compositions comprising CAR-expressing immune effector cells disclosed herein may be administered in conjunction with any number of chemotherapeutic agents. Illustrative examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®., Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on cancers such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A variety of other therapeutic agents may be used in conjunction with the compositions described herein. In one embodiment, the composition comprising CAR-expressing immune effector cells is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Other exemplary NSAIDs are chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

Illustrative examples of therapeutic antibodies suitable for combination with the CAR modified T cells contemplated in particular embodiments, include but are not limited to, bavituximab, bevacizumab (avastin), bivatuzumab, blinatumomab, conatumumab, daratumumab, duligotumab, dacetuzumab, dalotuzumab, elotuzumab (HuLuc63), gemtuzumab, ibritumomab, indatuximab, inotuzumab, lorvotuzumab, lucatumumab, milatuzumab, moxetumomab, ocaratuzumab, ofatumumab, rituximab, siltuximab, teprotumumab, and ublituximab.

In certain embodiments, the compositions described herein are administered in conjunction with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In particular embodiments, a composition comprises CAR T cells contemplated herein that are cultured in the presence of a PI3K inhibitor as disclosed herein and express one or more of the following markers: CD3, CD4, CD8, CD27, CD28, CD45RA, CD45RO, CD62L, CD127, and HLA-DR can be further isolated by positive or negative selection techniques. In one embodiment, a composition comprises a specific subpopulation of T cells, expressing one or more of the markers selected from the group consisting of i) CD62L, CCR7, CD28, CD27, CD122, CD127, CD197; ii) CD62L, CD127, CD197, CD38; and iii) CD62L, CD27, CD127, and CD8, is further isolated by positive or negative selection techniques. In various embodiments, compositions do not express or do not substantially express one or more of the following markers: CD57, CD244, CD160, PD-1, CTLA4, TIM3, and LAG3.

In one embodiment, expression of one or more of the markers selected from the group consisting of CD62L, CD127, CD197, and CD38 is increased at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, or more compared to a population of T cells activated and expanded without a PI3K inhibitor.

In one embodiment, expression of one or more of the markers selected from the group consisting of CD62L, CD127, CD27, and CD8 is increased at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, or more compared to a population of T cells activated and expanded without a PI3K inhibitor.

In one embodiment, expression of one or more of the markers selected from the group consisting of CD57, CD244, CD160, PD-1, CTLA4, TIM3, and LAG3 is decreased at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, or more compared to a population of T cells activated and expanded with a PI3K inhibitor.

### J. Targets Cells and Antigens

Genetically modified immune effector cells redirected to a target cell, e.g., cancer cell, and that comprise CARs having a binding domain that binds to ROR1 on the target cells are provided in particular embodiments. As used herein, the term “cancer” relates generally to a class of diseases or conditions in which abnormal cells divide without control and can invade nearby tissues.

As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” refers to an individual cell of a cancerous growth or tissue. Cancer cells include both solid cancers and liquid cancers. A “tumor” or “tumor cell” refers generally to a swelling or lesion formed by an abnormal growth of cells, which may be benign, pre-malignant, or malignant. Most cancers form tumors, but liquid cancers, e.g., leukemia, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

In one embodiment, the target cell expresses an antigen, e.g., a target antigen that is not substantially found on the surface of other normal (desired) cells.

In one embodiment, the target cell is a bone cell, osteocyte, osteoblast, adipose cell, chondrocyte, chondroblast, muscle cell, skeletal muscle cell, myoblast, myocyte, smooth muscle cell, bladder cell, bone marrow cell, central nervous system (CNS) cell, peripheral nervous system (PNS) cell, glial cell, astrocyte cell, neuron, pigment cell, epithelial cell, skin cell, endothelial cell, vascular endothelial cell, breast cell, colon cell, esophagus cell, gastrointestinal cell, stomach cell, colon cell, head cell, neck cell, gum cell, tongue cell, kidney cell, liver cell, lung cell, nasopharynx cell, ovary cell, follicular cell, cervical cell, vaginal cell, uterine cell, pancreatic cell, pancreatic parenchymal cell, pancreatic duct cell, pancreatic islet cell, prostate cell, penile cell, gonadal cell, testis cell, hematopoietic cell, lymphoid cell, or myeloid cell.

In one embodiment, the target cell is a hematopoietic cell, a skin cell, a breast cell, a lung cell, an adrenal cell, a bladder cell, a colon cell, a pancreatic cell, a prostate cell, a testicular cell, a uterine cell, an ovary cell, a follicular cell, an endothelial cell, an epithelial cell, a lymphoid cell, or a myeloid cell.

In certain embodiments, the target cell is part of the blood, a skin tissue, a breast tissue, a lung tissue, an adrenal tissue, a bladder tissue, a colon tissue, a pancreatic tissue, a prostate tissue, a testicular tissue, a uterine tissue, an ovarian tissue, a follicular tissue, an epithelial tissue, a lymphoid tissue, or a myeloid tissue.

In a particular embodiment, the target cell is a cancer cell or cancer stem cell that expresses ROR1.

In another embodiment, the target cell is a solid cancer cell that expresses ROR1.

Illustrative examples of ROR1 expressing solid tumor target cells that can be targeted by the compositions and methods contemplated in particular embodiments include, but are not limited to those of the following solid cancers: adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor.

In one embodiment, the ROR1 expressing target cell is an epithelial cell derived solid tumor.

In one embodiment, the ROR1 expressing target cell is a lung cancer cell, breast cancer cell, pancreatic cancer cell, ovarian cancer cell, prostate cancer cell, adrenal cancer cell, melanoma cancer cell, uterine cancer cell, testicular cancer cell, or bladder cancer cell.

In a particular embodiment, the target cell is a liquid cancer cell or hematological cancer cell that expresses ROR1.

Illustrative examples of liquid cancers or hematological cancers that may be prevented, treated, or ameliorated with the compositions contemplated in particular embodiments include, but are not limited to: leukemias, lymphomas, and multiple myeloma.

Illustrative examples of cells that can be targeted by anti-ROR1 CARS contemplated in particular embodiments include, but are not limited to those of the following leukemias: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CIVIL), chronic myelomonocytic leukemia (CMML) and polycythemia vera.

Illustrative examples of cells that can be targeted by the compositions and methods contemplated in particular embodiments include, but are not limited to those of the following lymphomas: Hodgkin lymphoma, nodular lymphocyte-predominant Hodgkin lymphoma and Non-Hodgkin lymphoma, including but not limited to B-cell non-Hodgkin lymphomas: Burkitt lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma; and T-cell non-Hodgkin lymphomas: mycosis fungoides, anaplastic large cell lymphoma, Sézary syndrome, and precursor T-lymphoblastic lymphoma.

Illustrative examples of cells that can be targeted by the compositions and methods contemplated in particular embodiments include, but are not limited to those of the following multiple myelomas: overt multiple myeloma, smoldering multiple myeloma (MGUS), plasma cell leukemia, non-secretory myeloma, IgD myeloma, osteosclerotic myeloma, solitary plasmacytoma of bone, and extramedullary plasmacytoma.

In one embodiment, the ROR1 expressing target cell is a CLL cancer cell or a mantle cell lymphoma cancer cell.

In a particular embodiment, the target cell is a cancer cell or cancer stem cell that expresses ROR1.

In another particular embodiment, the target cell is a cancer cell, such as a cell in a patient with cancer.

### K. Therapeutic Methods

The genetically modified immune effector cells contemplated herein provide improved methods of adoptive immunotherapy for use in the prevention, treatment, and amelioration cancers that express ROR1 or for preventing, treating, or ameliorating at least one symptom associated with an ROR1 expressing cancer.

In various embodiments, the genetically modified immune effector cells contemplated herein provide improved methods of adoptive immunotherapy for use in increasing the cytotoxicity in cancer cells that express ROR1 in a subject or for use in decreasing the number of cancer cells expressing ROR1 in a subject.

In particular embodiments, the specificity of a primary immune effector cell is redirected to cells expressing ROR1, e.g., cancer cells, by genetically modifying the primary immune effector cell with a CAR contemplated herein. In various embodiments, a viral vector is used to genetically modify an immune effector cell with a particular polynucleotide encoding a CAR comprising an anti-ROR1 antigen binding domain that binds an ROR1 polypeptide; a hinge domain; a transmembrane (TM) domain, a short oligo- or polypeptide linker, that links the TM domain to the intracellular signaling domain of the CAR; and one or more intracellular co-stimulatory signaling domains; and a primary signaling domain.

In one embodiment, a type of cellular therapy where T cells are genetically modified to express a CAR that targets ROR1 expressing cancer cells, and the CAR T cell is infused to a recipient in need thereof is provided. The infused cell is able to kill disease causing cells in the recipient. Unlike antibody therapies, CAR T cells are able to replicate in vivo resulting in long-term persistence that can lead to sustained cancer therapy.

In one embodiment, the CAR T cells can undergo robust in vivo T cell expansion and can persist for an extended amount of time. In another embodiment, the CAR T cells evolve into specific memory T cells that can be reactivated to inhibit any additional tumor formation or growth.

In particular embodiments, compositions comprising immune effector cells comprising the CARs contemplated herein are used in the treatment of conditions associated with ROR1 expressing cancer cells or cancer stem cells.

Illustrative examples of conditions that can be treated, prevented or ameliorated using the immune effector cells comprising the CARs contemplated in particular embodiments include, but are not limited to: a lung cancer, a breast cancer, a pancreatic cancer, and a bladder cancer.

In particular embodiments, compositions comprising CAR-modified T cells contemplated herein are used in the treatment of solid cancers. In certain embodiments, the solid cancer is selected from the group consisting of: lung cancer, breast cancer, pancreatic cancer, ovarian cancer, prostate cancer, adrenal cancer, melanoma, uterine cancer, testicular cancer, or bladder cancer.

In a particular embodiment, compositions comprising CAR-modified T cells contemplated herein are used in the treatment of liquid or hematological cancers.

In certain embodiments, the liquid or hematological cancer is selected from the group consisting of: leukemias, lymphomas, and multiple myelomas.

In certain embodiments, the liquid or hematological cancer is selected from the group consisting of: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CIVIL), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In certain embodiments, the liquid or hematological cancer is selected from the group consisting of: acute lymphocytic leukemia (ALL), chronic lymphocytic leukemia (CLL), hairy cell leukemia (HCL), multiple myeloma (MM), acute myeloid leukemia (AML), or chronic myeloid leukemia (CIVIL).

In certain embodiments, the liquid or hematological cancer is CLL or mantle cell lymphoma.

In particular embodiments, methods comprising administering a therapeutically effective amount of CAR-expressing immune effector cells contemplated herein or a composition comprising the same, to a patient in need thereof, alone or in combination with one or more therapeutic agents, are provided. In certain embodiments, the cells are used in the treatment of patients at risk for developing a condition associated with cancer cells that express ROR1. Thus, in particular embodiments, methods for the treatment or prevention or amelioration of at least one symptom of cancer comprising administering to a subject in need thereof, a therapeutically effective amount of the CAR-modified cells contemplated herein.

As used herein, the terms “individual” and “subject” are often used interchangeably and refer to any animal that exhibits a symptom of a disease, disorder, or condition that can be treated with the gene therapy vectors, cell-based therapeutics, and methods contemplated elsewhere herein. In preferred embodiments, a subject includes any animal that exhibits symptoms of a disease, disorder, or condition related to cancer that can be treated with the gene therapy vectors, cell-based therapeutics, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have an ROR1 expressing cancer, have been diagnosed with an ROR1 expressing cancer, or are at risk or having an ROR1 expressing cancer.

As used herein, the term “patient” refers to a subject that has been diagnosed with a particular disease, disorder, or condition that can be treated with the gene therapy vectors, cell-based therapeutics, and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

By “enhance” or “promote,” or “increase” or “expand” refers generally to the ability of a composition contemplated herein, e.g., a genetically modified T cell or vector encoding a CAR, to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a substantially similar or comparable physiological response (i.e., downstream effects) in a cell, as compared to the response caused by either vehicle, a control molecule/composition, or the response in a particular cell lineage. A comparable response is one that is not significantly different or measurable different from the reference response.

In one embodiment, a method of treating cancer in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In one embodiment, the amount of immune effector cells, e.g., T cells, in the composition administered to a subject is at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells.

In particular embodiments, about 1×10⁷ T cells to about 1×10⁹ T cells, about 2×10⁷ T cells to about 0.9×10⁹ T cells, about 3×10⁷ T cells to about 0.8×10⁹ T cells, about 4×10⁷ T cells to about 0.7×10⁹ T cells, about 5×10⁷ T cells to about 0.6×10⁹ T cells, or about 5×10⁷ T cells to about 0.5×10⁹ T cells are administered to a subject.

In one embodiment, the amount of immune effector cells, e.g., T cells, in the composition administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight.

In particular embodiments, about 1×10⁶ T cells/kg of bodyweight to about 1×10⁸ T cells/kg of bodyweight, about 2×10⁶ T cells/kg of bodyweight to about 0.9×10⁸ T cells/kg of bodyweight, about 3×10⁶ T cells/kg of bodyweight to about 0.8×10⁸ T cells/kg of bodyweight, about 4×10⁶ T cells/kg of bodyweight to about 0.7×10⁸ T cells/kg of bodyweight, about 5×10⁶ T cells/kg of bodyweight to about 0.6×10⁸ T cells/kg of bodyweight, or about 5×10⁶ T cells/kg of bodyweight to about 0.5×10⁸ T cells/kg of bodyweight are administered to a subject.

One of ordinary skill in the art would recognize that multiple administrations of the compositions contemplated herein may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In certain embodiments, it may be desirable to administer activated immune effector cells to a subject and then subsequently redraw blood (or have an apheresis performed), activate immune effector cells therefrom, and reinfuse the patient with these activated and expanded immune effector cells. This process can be carried out multiple times every few weeks. In certain embodiments, immune effector cells can be activated from blood draws of from 10 cc to 400 cc. In certain embodiments, immune effector cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or 400 cc or more. Not to be bound by theory, using this multiple blood draw/multiple reinfusion protocol may serve to select out certain populations of immune effector cells.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a subject in need thereof is administered an effective amount of a composition to increase a cellular immune response to a B cell related condition in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

In the case of T cell-mediated killing, CAR-ligand binding initiates CAR signaling to the T cell, resulting in activation of a variety of T cell signaling pathways that induce the T cell to produce or release proteins capable of inducing target cell apoptosis by various mechanisms. These T cell-mediated mechanisms include (but are not limited to) the transfer of intracellular cytotoxic granules from the T cell into the target cell, T cell secretion of pro-inflammatory cytokines that can induce target cell killing directly (or indirectly via recruitment of other killer effector cells), and up regulation of death receptor ligands (e.g. FasL) on the T cell surface that induce target cell apoptosis following binding to their cognate death receptor (e.g. Fas) on the target cell.

In one embodiment, a method of treating a subject diagnosed with an ROR1 expressing cancer is provided comprising removing immune effector cells from a subject diagnosed with an ROR1 expressing cancer, genetically modifying said immune effector cells with a vector comprising a nucleic acid encoding a CAR contemplated herein, thereby producing a population of modified immune effector cells, and administering the population of modified immune effector cells to the same subject. In a preferred embodiment, the immune effector cells comprise T cells.

In certain embodiments, methods for stimulating an immune effector cell mediated immune modulator response to a target cell population in a subject are provided comprising the steps of administering to the subject an immune effector cell population expressing a nucleic acid construct encoding a CAR molecule.

The methods for administering the cell compositions contemplated in particular embodiments includes any method which is effective to result in reintroduction of ex vivo genetically modified immune effector cells that either directly express a CAR in the subject or on reintroduction of the genetically modified progenitors of immune effector cells that on introduction into a subject differentiate into mature immune effector cells that express the CAR. One method comprises transducing peripheral blood T cells ex vivo with a nucleic acid construct contemplated herein and returning the transduced cells into the subject.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Construction of Anti-ROR1 CARs

CARs containing humanized anti-ROR1 scFv antibodies were designed to contain an MND promoter operably linked to anti-ROR1 scFv, a hinge and transmembrane domain from CD8α and a CD137 co-stimulatory domains followed by the intracellular signaling domain of the CD3 chain. FIG. 1. The anti-ROR1 CARs comprise a CD8α signal peptide (SP) sequence for the surface expression on immune effector cells. Table 3 shows the Identity, Genbank Reference, Source Name and Citation for the various nucleotide segments of an exemplary anti-ROR1 CAR lentiviral vector.

TABLE 3

| Nucleotides | Identity | GenBank Reference | Source Name | Citation |
|---|---|---|---|---|
| 1-185 | pUC19 plasmid | Accession #L09137.2 | pUC19 | New England |
| | backbone | nt 1-185 | | Biolabs |
| 185-222 | Linker | Not applicable | Synthetic | Not applicable |
| 223-800 | CMV | Not Applicable | pHCMV | (1994) PNAS |
| | | | | 91: 9564-68 |
| 801-1136 | R, U5, PBS, and | Accession #M19921.2 | pNL4-3 | Maldarelli, et. al. |
| | packaging sequences | nt 454-789 | | (1991) |
| | | | | J Virol: |
| | | | | 65(11): 5732-43 |
| 1137-1139 | Gag start codon (ATG) | Not Applicable | Synthetic | Not applicable |
| | changed to stop codon | | | |
| | (TAG) | | | |
| 1140-1240 | HIV-1 gag sequence | Accession #M19921.2 | pNL4-3 | Maldarelli, et. al. |
| | | nt 793-893 | | (1991) |
| | | | | J Virol: |
| | | | | 65(11): 5732-43 |
| 1241-1243 | HIV-1 gag sequence | Not Applicable | Synthetic | Not applicable |
| | changed to a second | | | |
| | stop codon | | | |
| 1244-1595 | HIV-1 gag sequence | Accession #M19921.2 | pNL4-3 | Maldarelli, et. al. |
| | | nt 897-1248 | | (1991) |
| | | | | J Virol: |
| | | | | 65(11): 5732-43 |
| 1596-1992 | HIV-1 pol | Accession #M19921.2 | pNL4-3 | Maldarelli, et. al. |
| | cPPT/CTS | nt 4745-5125 | | (1991) |
| | | | | J Virol: |
| | | | | 65(11): 5732-43 |
| 1993-2517 | HIV-1, isolate HXB3 | Accession #M14100.1 | PgTAT-CMV | Malim, M. H. |
| | env region (RRE) | nt 1875-2399 | | Nature (1988) |
| | | | | 335: 181-183 |
| 2518-2693 | HIV-1 env sequences | Accession #M19921.2 | pNL4-3 | Maldarelli, et. al. |
| | S/A | nt 8290-8470 | | (1991) |
| | | | | J Virol: |
| | | | | 65(11): 5732-43 |
| 2694-2708 | Linker | Not applicable | Synthetic | Not applicable |
| 2709-3096 | MND | Not applicable | rSPA.mPro.M | Challita et al. |
| | | | ND | (1995) |
| | | | | J. Virol. 69: |
| | | | | 748-755 |
| 3097-3125 | Linker | Not applicable | Synthetic | Not applicable |
| 3126-3188 | Signal peptide | | Synthetic | Not applicable |
| variable | Anti-ROR1 scFv | Not applicable | Synthetic | Not applicable |
| 3927-3935 | Linker | Not applicable | Synthetic | Not applicable |
| 3936-4142 | CD8a hinge and TM | Accession # | Synthetic | Milone et al |
| | | NM_001768 | | (2009) |
| | | | | Mol Ther |
| | | | | 17(8): 1453-64 |
| 4143-4268 | CD137 (4-1BB) | Accession # | Synthetic | Milone et al |
| | signaling domain | NM_001561 | | (2009) |
| | | | | Mol Ther |
| | | | | 17(8): 1453-64 |
| 4269-4607 | CD3-ζ signaling | Accession # | Synthetic | Milone et al |
| | domain | NM_000734 | | (2009) |
| | | | | Mol Ther |
| | | | | 17(8): 1453-64 |
| 4608-4718 | HIV-1 ppt and part of | Accession #M19921.2 | pNL4-3 | Maldarelli, et. al. |
| | U3 | nt 9005-9110 | | (1991) |
| | | | | J Virol: |
| | | | | 65(11): 5732-43 |
| 4719-4835 | HIV-1 part of U3 | Accession #M19921.2 | pNL4-3 | Maldarelli, et. al. |
| | (399 bp deletion) and | nt 9511-9627 | | (1991) |
| | R | | | J Virol: |
| | | | | 65(11): 5732-43 |
| 4836-4859 | Synthetic polyA | Not applicable | Synthetic | Levitt, N. Genes |
| | | | | & Dev (1989) |
| | | | | 3: 1019-1025 |
| 4860-4878 | Linker | Not applicable | Synthetic | Not Applicable¹ |
| 4879-7351 | pUC19 backbone | Accession #L09137.2 | pUC19 | New England |
| | | nt 2636-2686 | | Biolabs |
| | | | | (Attached) |

### Example 2

### Evaluation of Human Anti-ROR1 CAR T Cells

Chimeric antigen receptors (CAR) specific to Receptor Tyrosine Kinase-Like Orphan Receptor 1 (ROR1) (SEQ ID NOs: 386-389) were evaluated for transduction efficiency, CAR expression, and ROR1 biological activity.

Anti-ROR1 CAR molecules were constructed using sequences from scFvs isolated from a human phage display library. CAR T cells were generated after lentiviral transduction of primary human T cells. Four CAR candidates were selected for further studies after an initial high throughput screen comprised of in vitro assays that assess CAR expression and ROR1-specific T cell activity. Anti-ROR1 CAR2 comprises the variable chains set forth in SEQ ID NOs: 7 and 8; anti-ROR1 CAR4 comprises the variable chains set forth in SEQ ID NOs: 63 and 64; anti-ROR1 CARE comprises the variable chains set forth in SEQ ID NOs: 15 and 16; and anti-ROR1 CAR15 comprises the variable chains set forth in SEQ ID NOs: 159 and 160. The anti-ROR1 CART cells were further analyzed for transduction efficiency, CAR expression, and ROR1 biological activity.

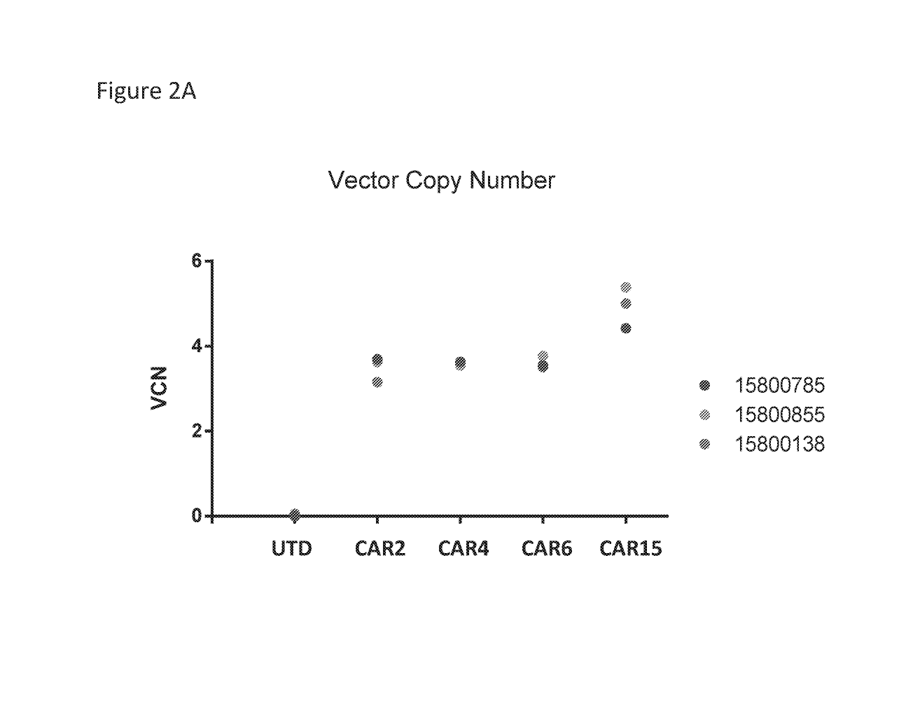

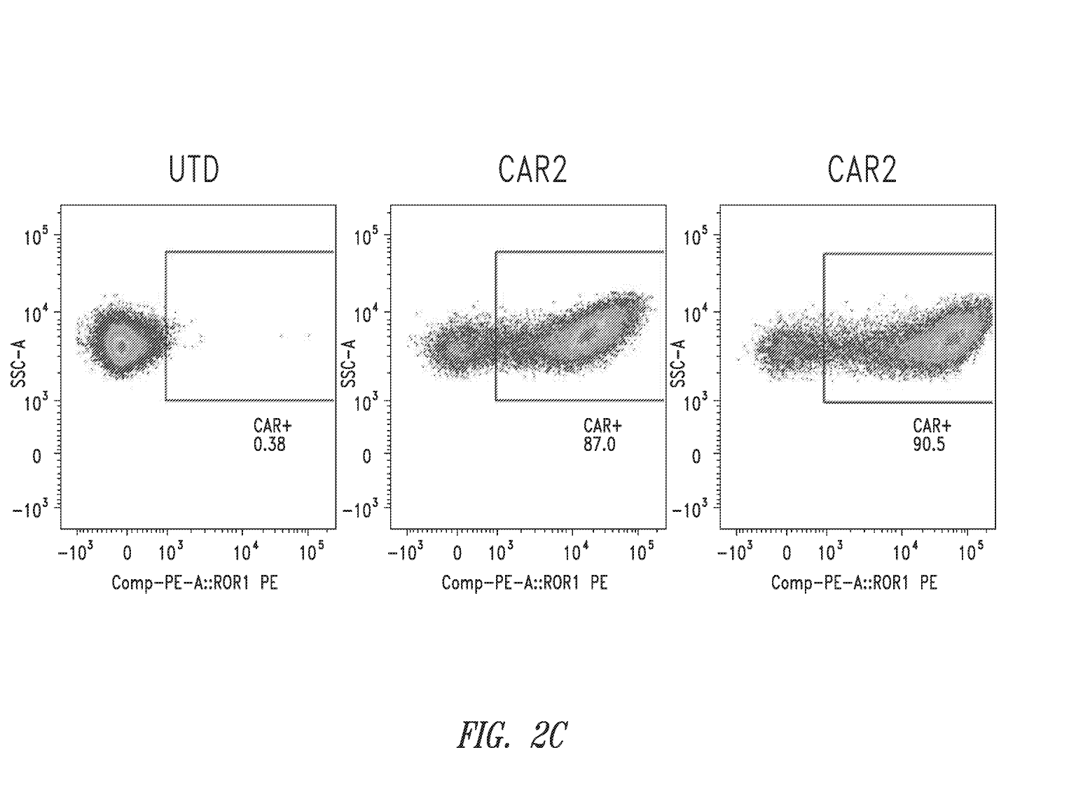

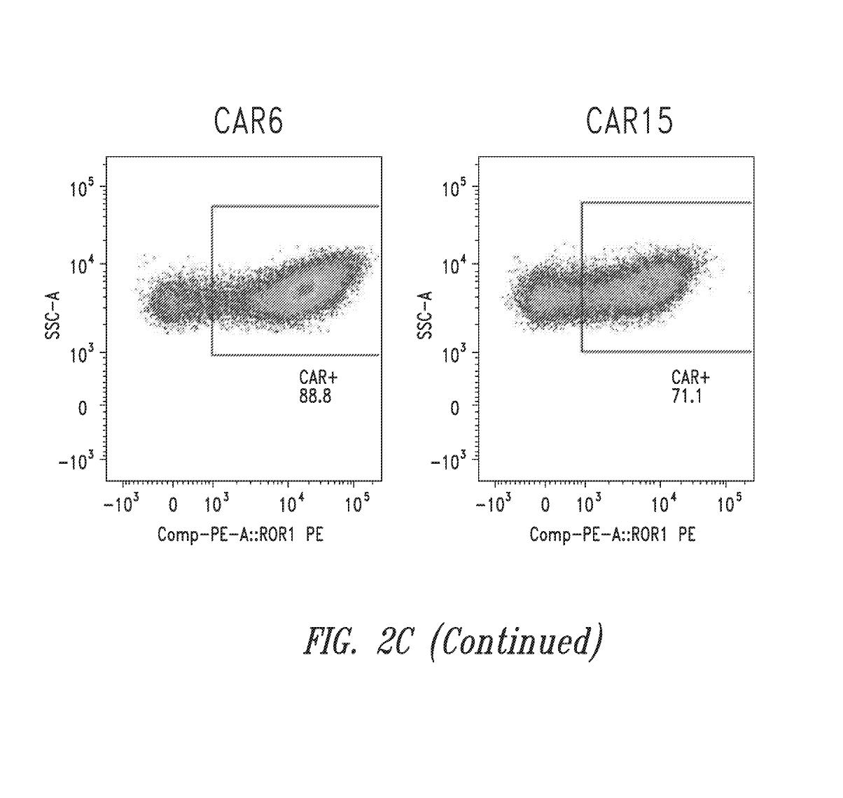

CAR T cells were produced using a system directly scalable to large clinical manufacturing processes. Briefly, peripheral blood mononuclear cells (PBMC) were cultured in static flasks in media containing IL-2 (CellGenix) and antibodies specific for CD3 and CD28 (Miltenyi Biotec). 2×10⁸ transducing units of lentivirus encoding anti-ROR1 CARs were added one day after culture initiation. The anti-ROR1 CAR T cells were maintained in log-phase by adding fresh media containing IL-2 for a total of ten days of culture. At the end of culture, the anti-ROR1 CAR T cells were interrogated for transduction efficiency. The number of integrated lentiviral particles was determined by q-PCR and presented as vector copy number (VCN). VCN from three primary T cell cultures processed in parallel are shown in FIG. 2A.

Expression of anti-ROR1 CAR on T cells was examined using flow cytometry. Primary human T cells engineered with lentiviruses expressing anti-ROR1 CARs were stained with recombinant human ROR1-IgG1-Fc conjugated to mouse anti-human IgG-PE (Southern Biotech). This reagent specifically identified T cells expressing anti-ROR1 CARs. A representative dot plot is shown in FIG. 2C and CAR expression from primary T cell cultures processed in parallel are shown in FIG. 2B.

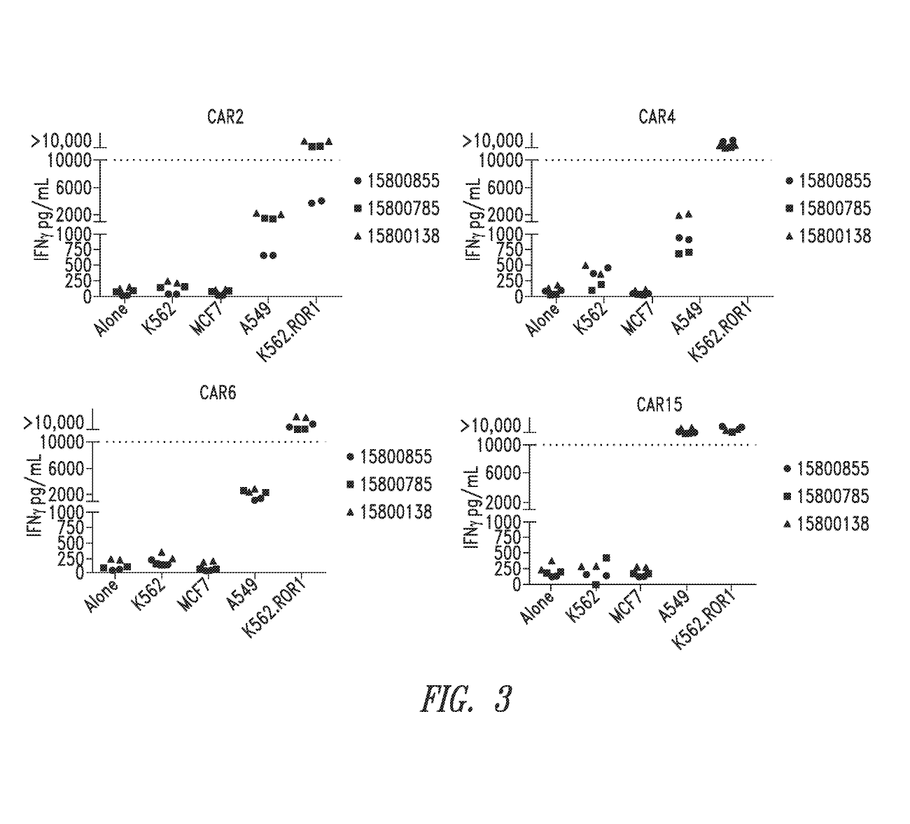

The biological activity of anti-ROR1 CAR T cells to ROR1-positive and ROR1-negative cell lines was assessed using an interferon-gamma (IFNγ) release assay. Anti-ROR1 CAR T cells were co cultured with K562 (ROR1⁻), MCF7 (ROR1⁻), A549 (ROR1⁺), or K562 (ROR1⁺) cell lines for 24 hours. Anti-ROR1 CAR T cells released IFNγ only in the presence of ROR1 positive cell lines. FIG. 3.

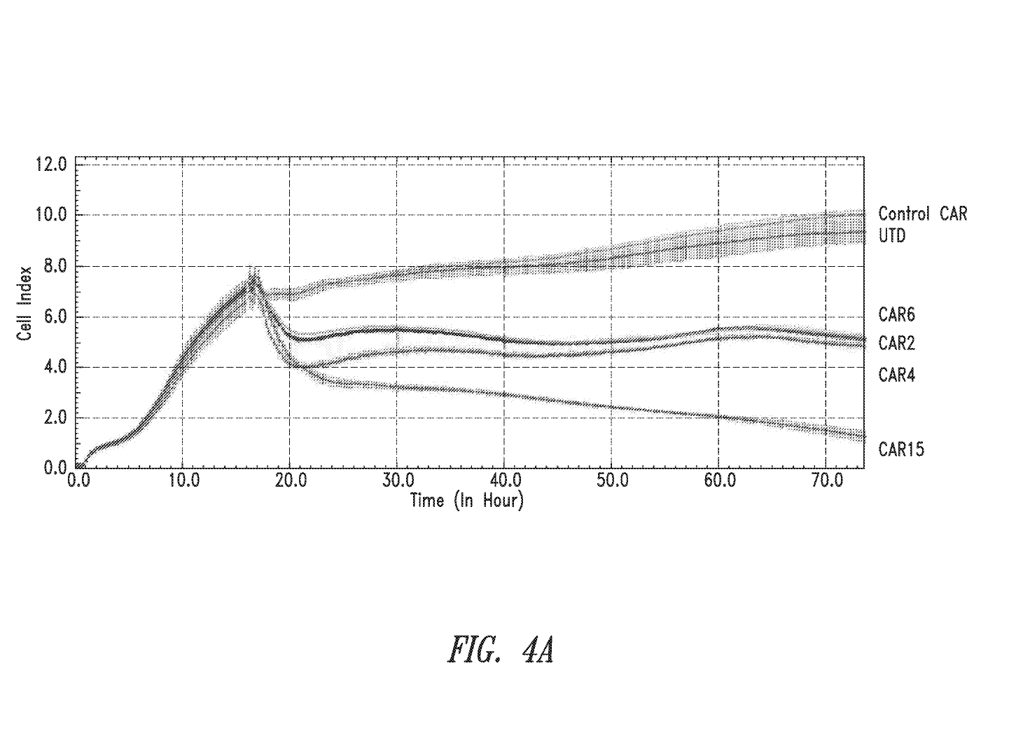

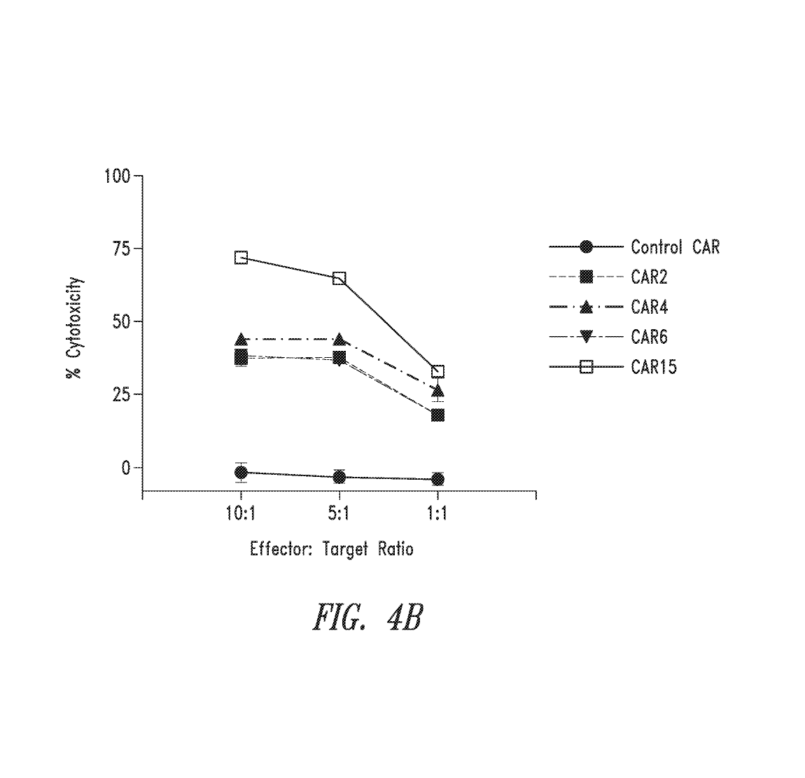

Cytolysis caused by anti-ROR1 CAR T cells was evaluated using co-culture assays. A549 (ROR1⁺) cell health after co-culture with anti-ROR1 or control CAR T cells was monitored real time with an iCELLigence instrument capable of monitoring electrical impedance of live cells. All anti-ROR1 CAR T cells caused cell death, the most cytotoxicity was associated with CAR15. FIGS. 4A and 4B.

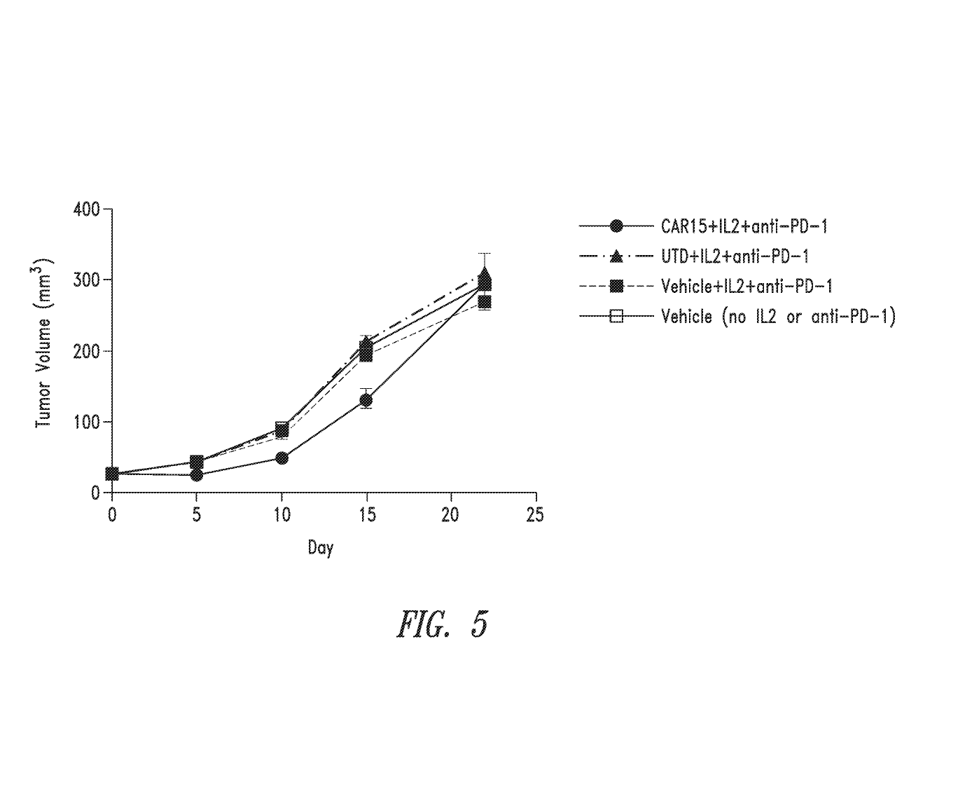

A mouse model of non-small cell lung cancer (NSCLC) was used to test the anti-tumor activity of the anti-ROR1 CART cells. NOD scid gamma (NSG) mice with ˜100 mm³ experimental sub-cutaneous human NSCLC (A549) tumors were treated with anti-ROR1 CAR15 T cells, untransduced T cells from the same donor, or vehicle (PBS). All treatment groups were supplemented with IL-2 (four days starting on the day of cell transfer) and an anti-PD-1 antibody (days 0, 6, and 12 after cell transfer). A549 growth was monitored with calipers. In two independent experiments, a measurable delay in tumor growth was observed. FIG. 5.

### Example 3

### Evaluation of Additional Human Anti-ROR1 CAR T Cells

Chimeric antigen receptors (CAR) specific to Receptor Tyrosine Kinase-Like Orphan Receptor 1 (ROR1) (SEQ ID NOs: 390-394) were evaluated for transduction efficiency, CAR expression, and ROR1 biological activity.

Anti-ROR1 CAR molecules were constructed using sequences from scFvs isolated from a human phage display library. CAR T cells were generated after lentiviral transduction of primary human T cells. Five CAR candidates were selected for further studies after an initial high throughput screen comprised of in vitro assays that assess CAR expression and ROR1-specific T cell activity. Anti-ROR1 CAR50 comprises the variable chains set forth in SEQ ID NOs: 255 and 256; anti-ROR1 CAR53 comprises the variable chains set forth in SEQ ID NOs: 271 and 272; anti-ROR1 CAR54 comprises the variable chains set forth in SEQ ID NOs: 279 and 280; anti-ROR1 CAR60 comprises the variable chains set forth in SEQ ID NOs: 319 and 320; and anti-ROR1 CAR66 comprises the variable chains set forth in SEQ ID NOs: 351 and 352. The anti-ROR1 CART cells were further analyzed for transduction efficiency, CAR expression, and ROR1 biological activity.

CAR T cells were produced using a system directly scalable to large clinical manufacturing processes. Briefly, peripheral blood mononuclear cells (PBMC) were cultured in static flasks in media containing IL-2 (CellGenix) and antibodies specific for CD3 and CD28 (Miltenyi Biotec). 2×10⁸ transducing units of lentivirus encoding anti-ROR1 CARs were added one day after culture initiation. The anti-ROR1 CAR T cells were maintained in log-phase by adding fresh media containing IL-2 for a total of ten days of culture. At the end of culture, the anti-ROR1 CAR T cells were interrogated for CAR expression and transduction efficiency.

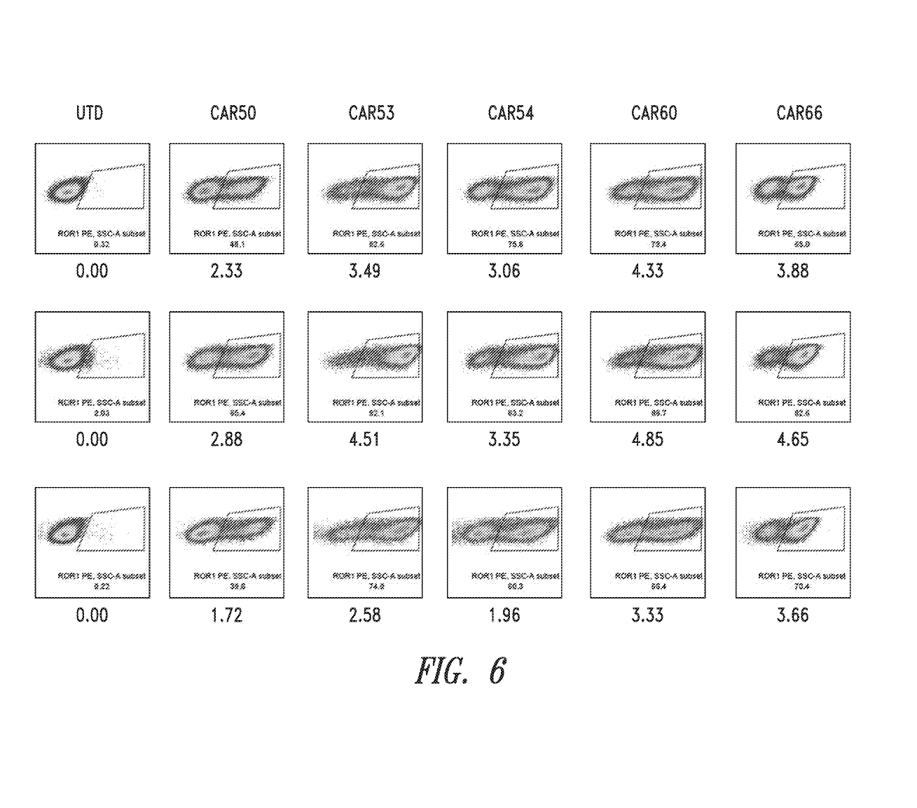

Expression of anti-ROR1 CAR on T cells was examined using flow cytometry. Primary human T cells engineered with lentiviruses expressing anti-ROR1 CARs were stained with recombinant human ROR1-IgG1-Fc conjugated to mouse anti-human IgG-PE (Southern Biotech). This reagent specifically identified T cells expressing anti-ROR1 CARs. A representative FACS plots showing CAR expression from primary T cell cultures processed in parallel are shown in FIG. 6.

The number of integrated lentiviral particles was determined by q-PCR and presented as vector copy number (VCN). VCN from three primary T cell cultures processed in parallel are shown in FIG. 6 (VCN values are values below each FACS plot).

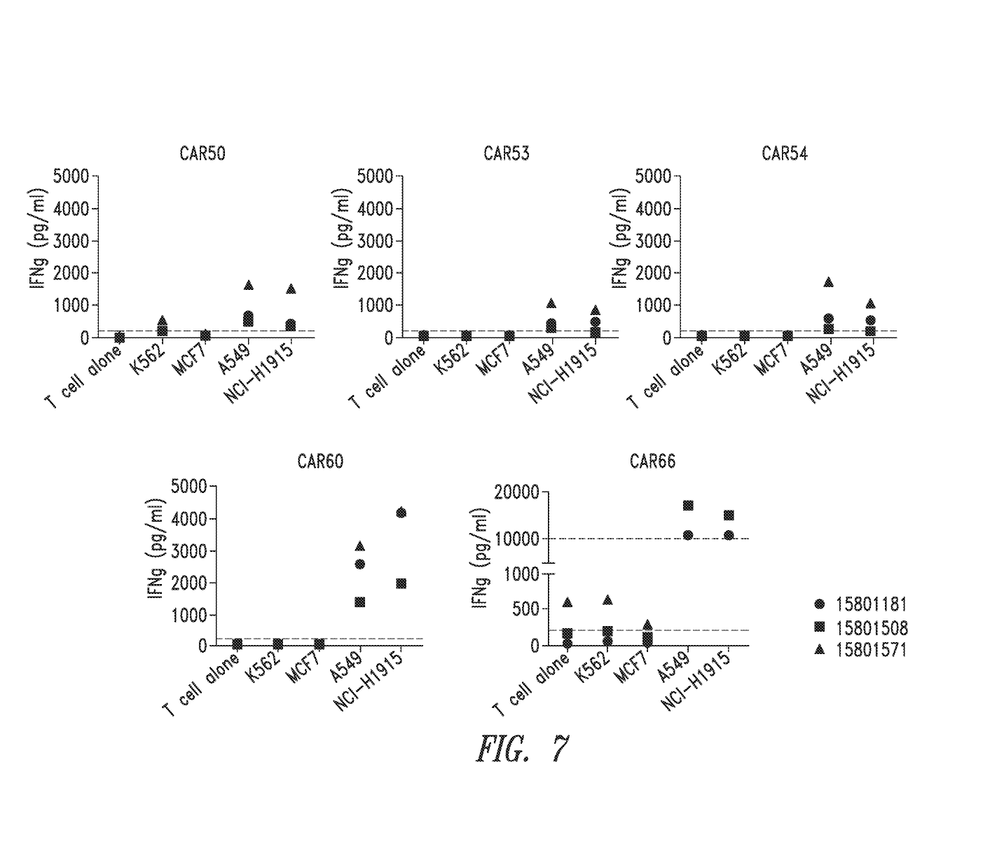

The biological activity of anti-ROR1 CAR T cells to ROR1-positive and ROR1-negative cell lines was assessed using an interferon-gamma (IFNγ) release assay. Anti-ROR1 CAR T cells were co cultured with vehicle, K562 (ROR1⁻), MCF7 (ROR1⁻), A549 (ROR1⁺), or NCI-H1915 (ROR1⁺) cell lines for 24 hours. Anti-ROR1 CAR T cells released IFNγ only in the presence of ROR1 positive cell lines. FIG. 7.

### Example 4

### Evaluation of Additional Human Anti-ROR1 CAR T Cells

Chimeric antigen receptors (CAR) specific to Receptor Tyrosine Kinase-Like Orphan Receptor 1 (ROR1) (SEQ ID NOs: 395-397) were evaluated for transduction efficiency, CAR expression, and ROR1 biological activity.

Anti-ROR1 CAR molecules were constructed using sequences from scFvs isolated from a human phage display library. CAR T cells were generated after lentiviral transduction of primary human T cells. Three CAR candidates were selected for further studies after an initial high throughput screen comprised of in vitro assays that assess CAR expression and ROR1-specific T cell activity. Anti-ROR1 CAR42 comprises the variable chains set forth in SEQ ID NOs: 199 and 200; anti-ROR1 CAR45 comprises the variable chains set forth in SEQ ID NOs: 103 and 104; and anti-ROR1 CAR46 comprises the variable chains set forth in SEQ ID NOs: 223 and 224. The anti-ROR1 CAR T cells were further analyzed for transduction efficiency, CAR expression, and ROR1 biological activity. Anti-ROR1 CAR2 was used as a positive control.

CAR T cells were produced using a system directly scalable to large clinical manufacturing processes. Briefly, peripheral blood mononuclear cells (PBMC) were cultured in static flasks in media containing IL-2 (CellGenix) and antibodies specific for CD3 and CD28 (Miltenyi Biotec). 2×10⁸ transducing units of lentivirus encoding anti-ROR1 CARs were added one day after culture initiation. The anti-ROR1 CAR T cells were maintained in log-phase by adding fresh media containing IL-2 for a total of ten days of culture. At the end of culture, the anti-ROR1 CAR T cells were interrogated for transduction efficiency and CAR expression.

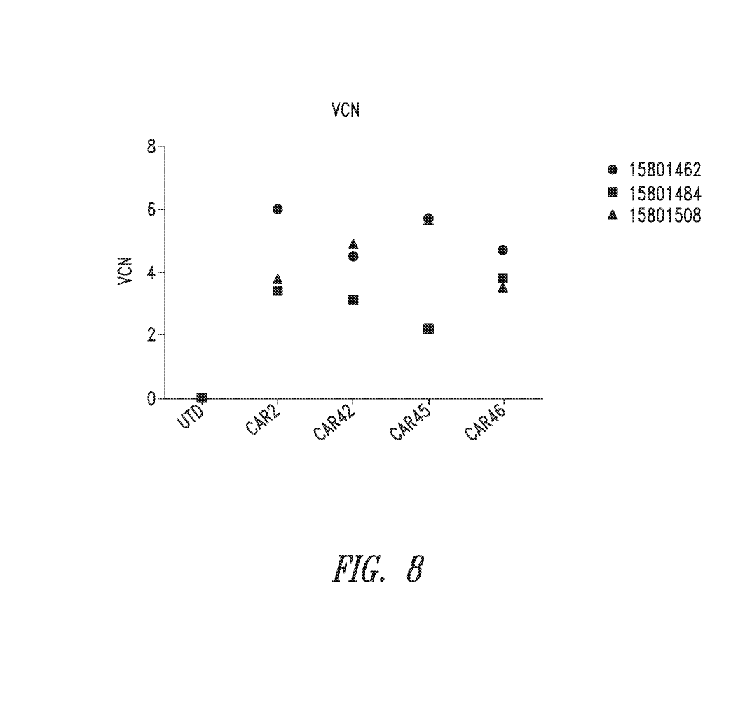

The number of integrated lentiviral particles was determined by q-PCR and presented as vector copy number (VCN). VCN from three primary T cell cultures processed in parallel are shown in FIG. 8.

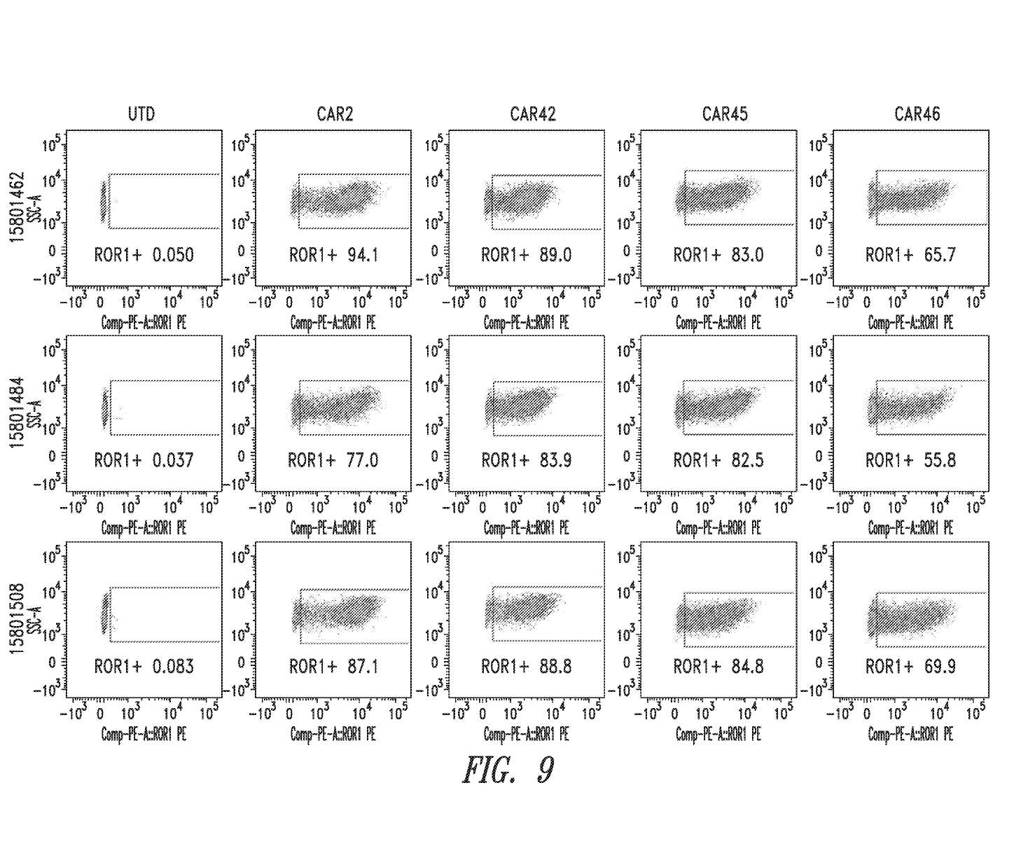

Expression of anti-ROR1 CAR on T cells was examined using flow cytometry. Primary human T cells engineered with lentiviruses expressing anti-ROR1 CARs were stained with recombinant human ROR1-IgG1-Fc conjugated to mouse anti-human IgG-PE (Southern Biotech). This reagent specifically identified T cells expressing anti-ROR1 CARs. A representative FACS plots showing CAR expression from primary T cell cultures processed in parallel are shown in FIG. 9.

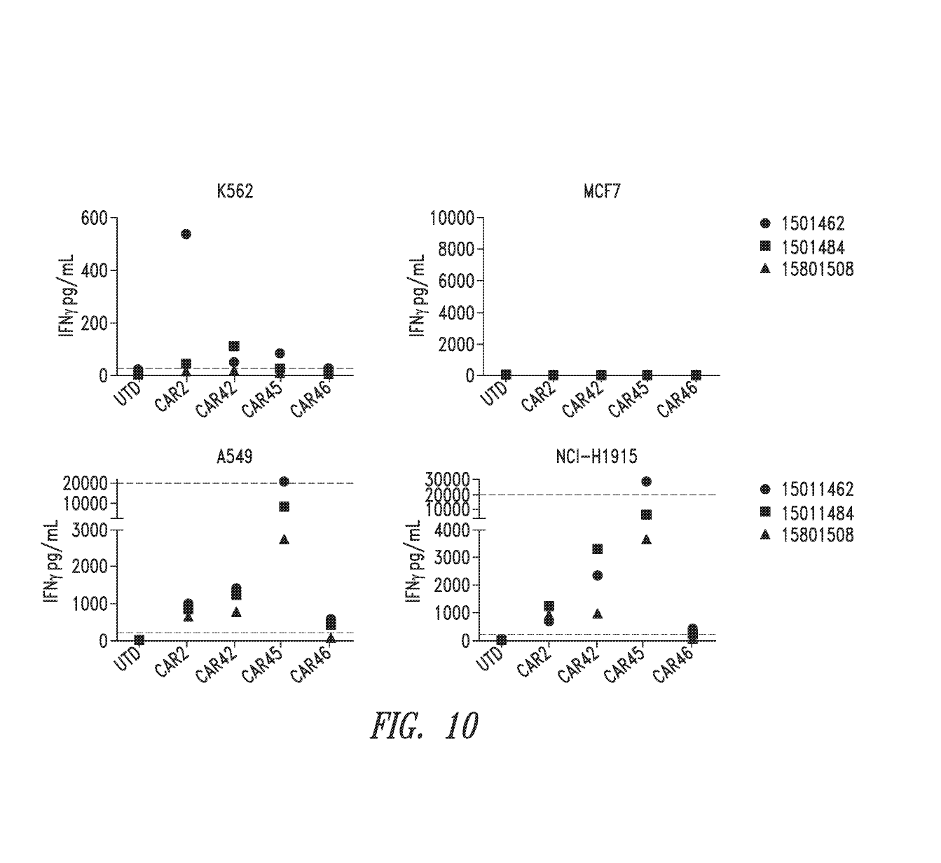

The biological activity of anti-ROR1 CAR T cells to ROR1-positive and ROR1-negative cell lines was assessed using an interferon-gamma (IFNγ) release assay. Anti-ROR1 CAR T cells were co cultured with vehicle, K562 (ROR1⁻; upper left panel), MCF7 (ROR1⁻; upper right panel), A549 (ROR1⁺; lower left panel), or NCI-H1915 (ROR1⁺; lower right panel) cell lines for 24 hours. Anti-ROR1 CAR T cells released IFNγ only in the presence of ROR1 positive cell lines. FIG. 10.

### Example 5

### Anti-ROR1 CAR T Cells Effect Antigen-Specific Cytotoxicity

Anti-ROR1 CAR T cells were produced using a system directly scalable to large clinical manufacturing processes. Briefly, peripheral blood mononuclear cells (PBMC) were cultured in static flasks in media containing IL-2 (CellGenix) and antibodies specific for CD3 and CD28 (Miltenyi Biotec). 2×10⁸ transducing units of lentivirus encoding anti-ROR1 CARs were added one day after culture initiation. The anti-ROR1 CAR T cells were maintained in log-phase by adding fresh media containing IL-2 for a total of ten days of culture. At the end of culture, the anti-ROR1 CAR T cells were assayed for antigen-specific cytotoxicity in two independent assays.

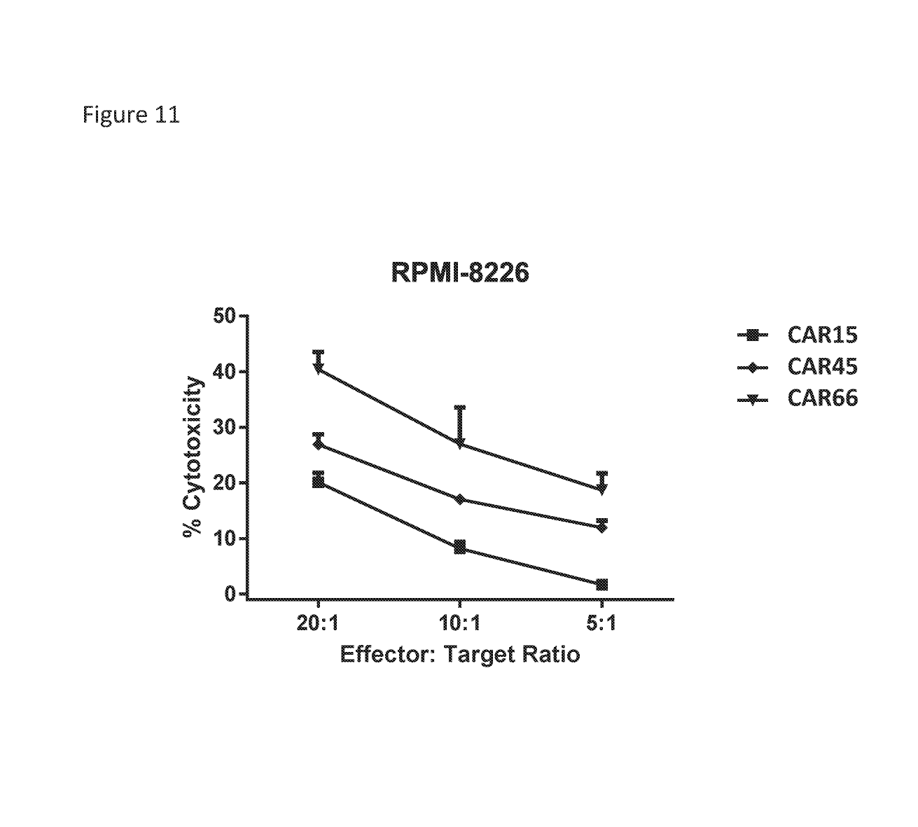

Antigen-specific cytotoxicity of ROR1⁺RPMI-8226 suspension tumor cells was assayed using a flow cytometric assay. RPMI-8226 tumor cells were labeled with CFSE fluorescent dye and mixed with an equal number of ROR1 negative K562 cells that were labeled with CellTrace Violet fluorescent dye. These target cells were then incubated with either untransduced (UTD) or anti-ROR1 CAR T cells (CAR15, CAR45, CAR66) at various effector:target cell ratios for 4 hours. The cytotoxicity of ROR1⁺ cells was measured by a decrease in the number of CFSE-labeled ROR1⁺ tumor cells relative to the CellTrace Violet-labeled ROR1 negative tumor cells after 4 hours of co-culture. FIG. 11.

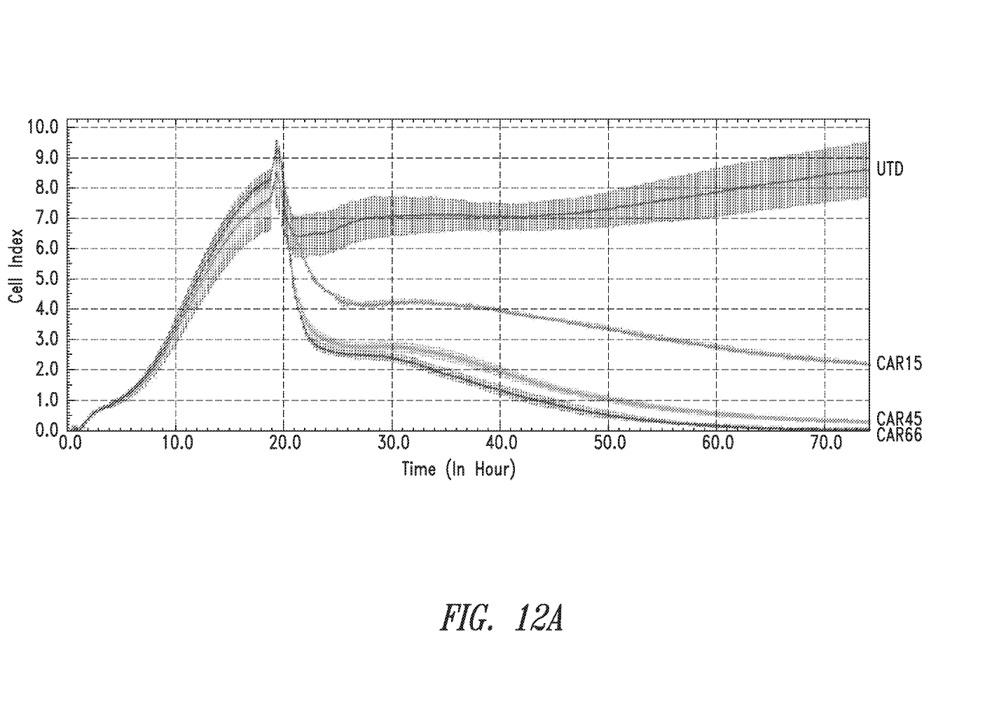

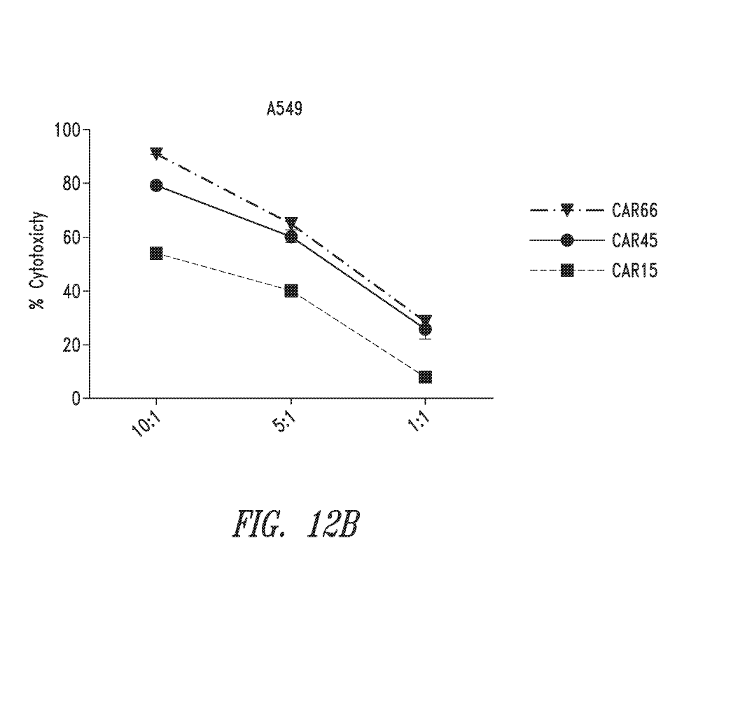

Cytotoxicity of ROR1⁺ A549 adherent tumor cells co-cultured with anti-ROR1 CAR T cells (CAR15, CAR45, CAR66) was monitored in real time with an iCELLigence instrument, which measures electrical impedance of live cells. A549 cells were seeded to a 96 well plate and cultured overnight while measuring impedance. The next day, untransduced (UTD) or anti-ROR1 CAR T cells were added at various effector:target ratios and impedance measurements were collected for a total of 55 more hours. Anti-ROR1 CAR T cells, but not untransduced cells, induced cytotoxicity of the A549 cells, as measured by a decreased impedance signal over time. FIG. 12A. The dose-dependent cytotoxicity of A549 cells co-cultured with anti-ROR1 CAR T cells at 10 hours is shown in FIG. 12B.

### Example 6

### Anti-ROR1 CAR T Cells in an In Vivo Tumor Model

Anti-ROR1 CAR T cells are produced using a system directly scalable to large clinical manufacturing processes. Briefly, peripheral blood mononuclear cells (PBMC) are cultured in static flasks in media containing IL-2 (CellGenix) and antibodies specific for CD3 and CD28 (Miltenyi Biotec). 2×10⁸ transducing units of lentivirus encoding anti-ROR1 CARs are added one day after culture initiation. The anti-ROR1 CAR T cells are maintained in log-phase by adding fresh media containing IL-2 for a total of ten days of culture. At the end of culture, the anti-ROR1 CAR T cells are assayed in an in vivo mantle cell lymphoma model.

JeKo-1 mantle lymphoma cells are labeled with a firefly luciferase gene and injected into NOD scid IL-2 receptor gamma chain knockout mice (NSG) by intravenous injection. After tumors are allowed to form, 1×10⁷ anti-ROR1 CAR T cells or untransduced control T cells are injected into tumor bearing mice. Tumor growth is monitored by bioluminescence using a Xenogen-IVIS Imaging system.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A chimeric antigen receptor (CAR) comprising: an extracellular domain that comprises:
a) an anti-receptor tyrosine kinase-like orphan receptor 1 (ROR1) antibody or antigen binding fragment thereof that binds one or more epitopes of a human ROR1 polypeptide, wherein the anti-ROR1 antibody or antigen binding fragment thereof comprises a variable light chain sequence comprising CDRL1-CDRL3 sequences set forth in SEQ ID NOs: 1-3, 9-11, 17-19, 25-27, 33-35, 41-43, 49-51, 57-59, 65-67, 73-75, 81-83, 89-91, 97-99, 105-107, 113-115, 121-123, 129-131, 137-139, 145-147, 153-155, 161-163, 169-171, 177-179, 185-187, 193-195, 201-203, 209-211, 217-219, 225-227, 233-235, 241-243, 249-251, 257-259, 265-267, 273-275, 281-283, 289-291, 297-299, 305-307, 313-315, 321-323, 329-331, 337-339, 345-347, or 353-355, and a variable heavy chain sequence comprising CDRH1-CDRH3 sequences set forth in SEQ ID NOs: 4-6, 12-14, 20-22, 28-30, 36-38, 44-46, 52-54, 60-62, 68-70, 76-78, 84-86, 92-94, 100-102, 108-110, 116-118, 124-126, 132-134, 140-142, 148-150, 156-158, 164-166, 172-174, 180-182, 188-190, 196-198, 204-206, 212-214, 220-222, 228-230, 236-238, 244-246, 252-254, 260-262, 268-270, 276-278, 284-286, 292-294, 300-302, 308-310, 316-318, 324-326, 332-334, 340-342, 348-350, or 356-358;
b) a transmembrane domain;
c) one or more intracellular co-stimulatory signaling domains; and
d) a primary signaling domain.

2. The CAR of claim 1, wherein the anti-ROR1 antibody or antigen binding fragment that binds the human ROR1 polypeptide is selected from the group consisting of: a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, Nanobody).

3. The CAR of claim 2, wherein the anti-ROR1 antibody or antigen binding fragment that binds the human ROR1 polypeptide is an scFv.

4. The CAR of any one of claims 1 to 3, wherein the anti-ROR1 antibody or antigen binding fragment thereof comprises one or more light chain CDRs as set forth in any one of SEQ ID NOs: 1-3 and/or one or more heavy chain CDRs as set forth in any one of SEQ ID NOs: 4-6.

5. The CAR of any one of claims 1 to 3, wherein the anti-ROR1 antibody or antigen binding fragment thereof comprises one or more light chain CDRs as set forth in any one of SEQ ID NOs: 9-11 and/or one or more heavy chain CDRs as set forth in any one of SEQ ID NOs: 12-14.

6. The CAR of any one of claims 1 to 3, wherein the anti-ROR1 antibody or antigen binding fragment thereof comprises one or more light chain CDRs as set forth in any one of SEQ ID NOs: 17-19 and/or one or more heavy chain CDRs as set forth in any one of SEQ ID NOs: 20-22.

7. The CAR of any one of claims 1 to 3, wherein the anti-ROR1 antibody or antigen binding fragment thereof comprises one or more light chain CDRs as set forth in any one of SEQ ID NOs: 25-27 and/or one or more heavy chain CDRs as set forth in any one of SEQ ID NOs: 28-30.

8. The CAR of any one of claims 1 to 3, wherein the anti-ROR1 antibody or antigen binding fragment thereof comprises one or more light chain CDRs as set forth in any one of SEQ ID NOs: 33-35 and/or one or more heavy chain CDRs as set forth in any one of SEQ ID NOs: 36-38.

9. The CAR of any one of claims 1 to 3, wherein the anti-ROR1 antibody or antigen binding fragment thereof comprises one or more light chain CDRs as set forth in any one of SEQ ID NOs: 41-43 and/or one or more heavy chain CDRs as set forth in any one of SEQ ID NOs: 44-46.

10. The CAR of any one of claims 1 to 3, wherein the anti-ROR1 antibody or antigen binding fragment thereof comprises one or more light chain CDRs as set forth in any one of SEQ ID NOs: 49-51 and/or one or more heavy chain CDRs as set forth in any one of SEQ ID NOs:
52-54.

11. The CAR of any one of claims 1 to 3, wherein the anti-ROR1 antibody or antigen binding fragment thereof comprises one or more light chain CDRs as set forth in any one of SEQ ID NOs: 57-59 and/or one or more heavy chain CDRs as set forth in any one of SEQ ID NOs: 60-62.

12. The CAR of any one of claims 1 to 3, wherein the anti-ROR1 antibody or antigen binding fragment thereof comprises one or more light chain CDRs as set forth in any one of SEQ ID NOs: 65-67 and/or one or more heavy chain CDRs as set forth in any one of SEQ ID NOs: 68-70.

13. The CAR of any one of claims 1 to 3, wherein the anti-ROR1 antibody or antigen binding fragment thereof comprises one or more light chain CDRs as set forth in any one of SEQ ID NOs: 73-75 and/or one or more heavy chain CDRs as set forth in any one of SEQ ID NOs: 76-78.

14. The CAR of any one of claims 1 to 3, wherein the anti-ROR1 antibody or antigen binding fragment thereof comprises one or more light chain CDRs as set forth in any one of SEQ ID NOs: 81-83 and/or one or more heavy chain CDRs as set forth in any one of SEQ ID NOs: 84-86.

15. The CAR of any one of claims 1 to 3, wherein the anti-ROR1 antibody or antigen binding fragment thereof comprises one or more light chain CDRs as set forth in any one of SEQ ID NOs: 89-91 and/or one or more heavy chain CDRs as set forth in any one of SEQ ID NOs: 92-94.

16. The CAR of any one of claims 1 to 3, wherein the anti-ROR1 antibody or antigen binding fragment thereof comprises one or more light chain CDRs as set forth in any one of SEQ ID NOs: 97-99 and/or one or more heavy chain CDRs as set forth in any one of SEQ ID NOs: 100-102.

17. The CAR of any one of claims 1 to 3, wherein the anti-ROR1 antibody or antigen binding fragment thereof comprises one or more light chain CDRs as set forth in any one of SEQ ID NOs: 105-107 and/or one or more heavy chain CDRs as set forth in any one of SEQ ID NOs: 108-110.

18. The CAR of any one of claims 1 to 3, wherein the anti-ROR1 antibody or antigen binding fragment thereof comprises one or more light chain CDRs as set forth in any one of SEQ ID NOs: 113-115 and/or one or more heavy chain CDRs as set forth in any one of SEQ ID NOs: 116-118.

19. The CAR of any one of claims 1 to 3, wherein the anti-ROR1 antibody or antigen binding fragment thereof comprises one or more light chain CDRs as set forth in any one of SEQ ID NOs: 121-123 and/or one or more heavy chain CDRs as set forth in any one of SEQ ID NOs: 124-126.

20. The CAR of any one of claims 1 to 3, wherein the anti-ROR1 antibody or antigen binding fragment thereof comprises one or more light chain CDRs as set forth in any one of SEQ ID NOs: 129-131 and/or one or more heavy chain CDRs as set forth in any one of SEQ ID NOs: 132-134.

21. The CAR of any one of claims 1 to 3, wherein the anti-ROR1 antibody or antigen binding fragment thereof comprises one or more light chain CDRs as set forth in any one of SEQ ID NOs: 137-139 and/or one or more heavy chain CDRs as set forth in any one of SEQ ID NOs: 140-142.

22. The CAR of any one of claims 1 to 3, wherein the anti-ROR1 antibody or antigen binding fragment thereof comprises one or more light chain CDRs as set forth in any one of SEQ ID NOs: 145-147 and/or one or more heavy chain CDRs as set forth in any one of SEQ ID NOs: 148-150.

23. The CAR of any one of claims 1 to 3, wherein the anti-ROR1 antibody or antigen binding fragment thereof comprises one or more light chain CDRs as set forth in any one of SEQ ID NOs: 153-155 and/or one or more heavy chain CDRs as set forth in any one of SEQ ID NOs: 156-158.

24. The CAR of any one of claims 1 to 3, wherein the anti-ROR1 antibody or antigen binding fragment thereof comprises one or more light chain CDRs as set forth in any one of SEQ ID NOs: 161-163 and/or one or more heavy chain CDRs as set forth in any one of SEQ ID NOs: 164-166.

25. The CAR of any one of claims 1 to 3, wherein the anti-ROR1 antibody or antigen binding fragment thereof comprises one or more light chain CDRs as set forth in any one of SEQ ID NOs: 169-171 and/or one or more heavy chain CDRs as set forth in any one of SEQ ID NOs: 172-174.

26. The CAR of any one of claims 1 to 3, wherein the anti-ROR1 antibody or antigen binding fragment thereof comprises one or more light chain CDRs as set forth in any one of SEQ ID NOs: 177-179 and/or one or more heavy chain CDRs as set forth in any one of SEQ ID NOs: 180-182.

27. The CAR of any one of claims 1 to 3, wherein the anti-ROR1 antibody or antigen binding fragment thereof comprises one or more light chain CDRs as set forth in any one of SEQ ID NOs: 185-187 and/or one or more heavy chain CDRs as set forth in any one of SEQ ID NOs: 188-190.

28. The CAR of any one of claims 1 to 3, wherein the anti-ROR1 antibody or antigen binding fragment thereof comprises one or more light chain CDRs as set forth in any one of SEQ ID NOs: 193-195 and/or one or more heavy chain CDRs as set forth in any one of SEQ ID NOs: 196-198.

29. The CAR of any one of claims 1 to 3, wherein the anti-ROR1 antibody or antigen binding fragment thereof comprises one or more light chain CDRs as set forth in any one of SEQ ID NOs: 201-203 and/or one or more heavy chain CDRs as set forth in any one of SEQ ID NOs: 204-206.

30. The CAR of any one of claims 1 to 3, wherein the anti-ROR1 antibody or antigen binding fragment thereof comprises one or more light chain CDRs as set forth in any one of SEQ ID NOs: 209-211 and/or one or more heavy chain CDRs as set forth in any one of SEQ ID NOs: 212-214.

31. The CAR of any one of claims 1 to 3, wherein the anti-ROR1 antibody or antigen binding fragment thereof comprises one or more light chain CDRs as set forth in any one of SEQ ID NOs: 217-219 and/or one or more heavy chain CDRs as set forth in any one of SEQ ID NOs: 220-222.

32. The CAR of any one of claims 1 to 3, wherein the anti-ROR1 antibody or antigen binding fragment thereof comprises one or more light chain CDRs as set forth in any one of SEQ ID NOs: 225-227 and/or one or more heavy chain CDRs as set forth in any one of SEQ ID NOs: 228-230.

33. The CAR of any one of claims 1 to 3, wherein the anti-ROR1 antibody or antigen binding fragment thereof comprises one or more light chain CDRs as set forth in any one of SEQ ID NOs: 233-235 and/or one or more heavy chain CDRs as set forth in any one of SEQ ID NOs: 236-238.

34. The CAR of any one of claims 1 to 3, wherein the anti-ROR1 antibody or antigen binding fragment thereof comprises one or more light chain CDRs as set forth in any one of SEQ ID NOs: 241-243 and/or one or more heavy chain CDRs as set forth in any one of SEQ ID NOs: 244-246.

35. The CAR of any one of claims 1 to 3, wherein the anti-ROR1 antibody or antigen binding fragment thereof comprises one or more light chain CDRs as set forth in any one of SEQ ID NOs: 249-251 and/or one or more heavy chain CDRs as set forth in any one of SEQ ID NOs: 252-254.

36. The CAR of any one of claims 1 to 3, wherein the anti-ROR1 antibody or antigen binding fragment thereof comprises one or more light chain CDRs as set forth in any one of SEQ ID NOs: 257-259 and/or one or more heavy chain CDRs as set forth in any one of SEQ ID NOs: 260-262.

37. The CAR of any one of claims 1 to 3, wherein the anti-ROR1 antibody or antigen binding fragment thereof comprises one or more light chain CDRs as set forth in any one of SEQ ID NOs: 265-267 and/or one or more heavy chain CDRs as set forth in any one of SEQ ID NOs: 268-270.

38. The CAR of any one of claims 1 to 3, wherein the anti-ROR1 antibody or antigen binding fragment thereof comprises one or more light chain CDRs as set forth in any one of SEQ ID NOs: 273-275 and/or one or more heavy chain CDRs as set forth in any one of SEQ ID NOs: 276-278.

39. The CAR of any one of claims 1 to 3, wherein the anti-ROR1 antibody or antigen binding fragment thereof comprises one or more light chain CDRs as set forth in any one of SEQ ID NOs: 281-283 and/or one or more heavy chain CDRs as set forth in any one of SEQ ID NOs: 284-286.

40. The CAR of any one of claims 1 to 3, wherein the anti-ROR1 antibody or antigen binding fragment thereof comprises one or more light chain CDRs as set forth in any one of SEQ ID NOs: 289-291 and/or one or more heavy chain CDRs as set forth in any one of SEQ ID NOs: 292-294.

41. The CAR of any one of claims 1 to 3, wherein the anti-ROR1 antibody or antigen binding fragment thereof comprises one or more light chain CDRs as set forth in any one of SEQ ID NOs: 297-299 and/or one or more heavy chain CDRs as set forth in any one of SEQ ID NOs: 300-302.

42. The CAR of any one of claims 1 to 3, wherein the anti-ROR1 antibody or antigen binding fragment thereof comprises one or more light chain CDRs as set forth in any one of SEQ ID NOs: 305-307 and/or one or more heavy chain CDRs as set forth in any one of SEQ ID NOs: 308-310.

43. The CAR of any one of claims 1 to 3, wherein the anti-ROR1 antibody or antigen binding fragment thereof comprises one or more light chain CDRs as set forth in any one of SEQ ID NOs: 313-315 and/or one or more heavy chain CDRs as set forth in any one of SEQ ID NOs: 316-318.

44. The CAR of any one of claims 1 to 3, wherein the anti-ROR1 antibody or antigen binding fragment thereof comprises one or more light chain CDRs as set forth in any one of SEQ ID NOs: 321-323 and/or one or more heavy chain CDRs as set forth in any one of SEQ ID NOs: 324-326.

45. The CAR of any one of claims 1 to 3, wherein the anti-ROR1 antibody or antigen binding fragment thereof comprises one or more light chain CDRs as set forth in any one of SEQ ID NOs: 329-331 and/or one or more heavy chain CDRs as set forth in any one of SEQ ID NOs: 332-334.

46. The CAR of any one of claims 1 to 3, wherein the anti-ROR1 antibody or antigen binding fragment thereof comprises one or more light chain CDRs as set forth in any one of SEQ ID NOs: 337-339 and/or one or more heavy chain CDRs as set forth in any one of SEQ ID NOs: 340-342.

47. The CAR of any one of claims 1 to 3, wherein the anti-ROR1 antibody or antigen binding fragment thereof comprises one or more light chain CDRs as set forth in any one of SEQ ID NOs: 345-347 and/or one or more heavy chain CDRs as set forth in any one of SEQ ID NOs: 348-350.

48. The CAR of any one of claims 1 to 3, wherein the anti-ROR1 antibody or antigen binding fragment thereof comprises one or more light chain CDRs as set forth in any one of SEQ ID NOs: 353-355 and/or one or more heavy chain CDRs as set forth in any one of SEQ ID NOs: 356-358.

49. The CAR of any one of claims 1 to 3, wherein the anti-ROR1 antibody or antigen binding fragment thereof comprises a variable light chain sequence as set forth in any one of SEQ ID NOs: 7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, 311, 319, 327, 335, 343, 351, or 359 and/or a variable heavy chain sequence as set forth in any one of SEQ ID NOs: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304, 312, 320, 328, 336, 344, 352, and 360.

50. The CAR of any one of claims 1 to 3, wherein the anti-ROR1 antibody or antigen binding fragment thereof comprises a variable light chain sequence as set forth in SEQ ID NO: 7 and/or a variable heavy chain sequence as set forth in SEQ ID NO: 8.

51. The CAR of any one of claims 1 to 3, wherein the anti-ROR1 antibody or antigen binding fragment thereof comprises a variable light chain sequence as set forth in SEQ ID NO: 15 and/or a variable heavy chain sequence as set forth in SEQ ID NO: 16.

52. The CAR of any one of claims 1 to 3, wherein the anti-ROR1 antibody or antigen binding fragment thereof comprises a variable light chain sequence as set forth in SEQ ID NO: 23 and/or a variable heavy chain sequence as set forth in SEQ ID NO: 24.

53. The CAR of any one of claims 1 to 3, wherein the anti-ROR1 antibody or antigen binding fragment thereof comprises a variable light chain sequence as set forth in SEQ ID NO: 31 and/or a variable heavy chain sequence as set forth in SEQ ID NO: 32.

54. The CAR of any one of claims 1 to 3, wherein the anti-ROR1 antibody or antigen binding fragment thereof comprises a variable light chain sequence as set forth in SEQ ID NO: 39 and/or a variable heavy chain sequence as set forth in SEQ ID NO: 40.

55. The CAR of any one of claims 1 to 3, wherein the anti-ROR1 antibody or antigen binding fragment thereof comprises a variable light chain sequence as set forth in SEQ ID NO: 47 and/or a variable heavy chain sequence as set forth in SEQ ID NO: 48.

56. The CAR of any one of claims 1 to 3, wherein the anti-ROR1 antibody or antigen binding fragment thereof comprises a variable light chain sequence as set forth in SEQ ID NO: 55 and/or a variable heavy chain sequence as set forth in SEQ ID NO: 56.

57. The CAR of any one of claims 1 to 3, wherein the anti-ROR1 antibody or antigen binding fragment thereof comprises a variable light chain sequence as set forth in SEQ ID NO: 63 and/or a variable heavy chain sequence as set forth in SEQ ID NO: 64.

58. The CAR of any one of claims 1 to 3, wherein the anti-ROR1 antibody or antigen binding fragment thereof comprises a variable light chain sequence as set forth in SEQ ID NO: 71 and/or a variable heavy chain sequence as set forth in SEQ ID NO: 72.

59. The CAR of any one of claims 1 to 3, wherein the anti-ROR1 antibody or antigen binding fragment thereof comprises a variable light chain sequence as set forth in SEQ ID NO: 79 and/or a variable heavy chain sequence as set forth in SEQ ID NO: 80.

60. The CAR of any one of claims 1 to 3, wherein the anti-ROR1 antibody or antigen binding fragment thereof comprises a variable light chain sequence as set forth in SEQ ID NO: 87 and/or a variable heavy chain sequence as set forth in SEQ ID NO: 88.

61. The CAR of any one of claims 1 to 3, wherein the anti-ROR1 antibody or antigen binding fragment thereof comprises a variable light chain sequence as set forth in SEQ ID NO: 95 and/or a variable heavy chain sequence as set forth in SEQ ID NO: 96.

62. The CAR of any one of claims 1 to 3, wherein the anti-ROR1 antibody or antigen binding fragment thereof comprises a variable light chain sequence as set forth in SEQ ID NO: 103 and/or a variable heavy chain sequence as set forth in SEQ ID NO: 104.

63. The CAR of any one of claims 1 to 3, wherein the anti-ROR1 antibody or antigen binding fragment thereof comprises a variable light chain sequence as set forth in SEQ ID NO: 111 and/or a variable heavy chain sequence as set forth in SEQ ID NO: 112.

64. The CAR of any one of claims 1 to 3, wherein the anti-ROR1 antibody or antigen binding fragment thereof comprises a variable light chain sequence as set forth in SEQ ID NO: 119 and/or a variable heavy chain sequence as set forth in SEQ ID NO: 120.

65. The CAR of any one of claims 1 to 3, wherein the anti-ROR1 antibody or antigen binding fragment thereof comprises a variable light chain sequence as set forth in SEQ ID NO: 127 and/or a variable heavy chain sequence as set forth in SEQ ID NO: 128.

66. The CAR of any one of claims 1 to 3, wherein the anti-ROR1 antibody or antigen binding fragment thereof comprises a variable light chain sequence as set forth in SEQ ID NO: 135 and/or a variable heavy chain sequence as set forth in SEQ ID NO: 136.

67. The CAR of any one of claims 1 to 3, wherein the anti-ROR1 antibody or antigen binding fragment thereof comprises a variable light chain sequence as set forth in SEQ ID NO: 143 and/or a variable heavy chain sequence as set forth in SEQ ID NO: 144.

68. The CAR of any one of claims 1 to 3, wherein the anti-ROR1 antibody or antigen binding fragment thereof comprises a variable light chain sequence as set forth in SEQ ID NO: 151 and/or a variable heavy chain sequence as set forth in SEQ ID NO: 152.

69. The CAR of any one of claims 1 to 3, wherein the anti-ROR1 antibody or antigen binding fragment thereof comprises a variable light chain sequence as set forth in SEQ ID NO: 159 and/or a variable heavy chain sequence as set forth in SEQ ID NO: 160.

70. The CAR of any one of claims 1 to 3, wherein the anti-ROR1 antibody or antigen binding fragment thereof comprises a variable light chain sequence as set forth in SEQ ID NO: 167 and/or a variable heavy chain sequence as set forth in SEQ ID NO: 168.

71. The CAR of any one of claims 1 to 3, wherein the anti-ROR1 antibody or antigen binding fragment thereof comprises a variable light chain sequence as set forth in SEQ ID NO: 175 and/or a variable heavy chain sequence as set forth in SEQ ID NO: 176.

72. The CAR of any one of claims 1 to 3, wherein the anti-ROR1 antibody or antigen binding fragment thereof comprises a variable light chain sequence as set forth in SEQ ID NO: 183 and/or a variable heavy chain sequence as set forth in SEQ ID NO: 184.

73. The CAR of any one of claims 1 to 3, wherein the anti-ROR1 antibody or antigen binding fragment thereof comprises a variable light chain sequence as set forth in SEQ ID NO: 191 and/or a variable heavy chain sequence as set forth in SEQ ID NO: 192.

74. The CAR of any one of claims 1 to 3, wherein the anti-ROR1 antibody or antigen binding fragment thereof comprises a variable light chain sequence as set forth in SEQ ID NO: 199 and/or a variable heavy chain sequence as set forth in SEQ ID NO: 200.

75. The CAR of any one of claims 1 to 3, wherein the anti-ROR1 antibody or antigen binding fragment thereof comprises a variable light chain sequence as set forth in SEQ ID NO: 207 and/or a variable heavy chain sequence as set forth in SEQ ID NO: 208.

76. The CAR of any one of claims 1 to 3, wherein the anti-ROR1 antibody or antigen binding fragment thereof comprises a variable light chain sequence as set forth in SEQ ID NO: 215 and/or a variable heavy chain sequence as set forth in SEQ ID NO: 216.

77. The CAR of any one of claims 1 to 3, wherein the anti-ROR1 antibody or antigen binding fragment thereof comprises a variable light chain sequence as set forth in SEQ ID NO: 223 and/or a variable heavy chain sequence as set forth in SEQ ID NO: 224.

78. The CAR of any one of claims 1 to 3, wherein the anti-ROR1 antibody or antigen binding fragment thereof comprises a variable light chain sequence as set forth in SEQ ID NO: 231 and/or a variable heavy chain sequence as set forth in SEQ ID NO: 232.

79. The CAR of any one of claims 1 to 3, wherein the anti-ROR1 antibody or antigen binding fragment thereof comprises a variable light chain sequence as set forth in SEQ ID NO: 239 and/or a variable heavy chain sequence as set forth in SEQ ID NO: 240.

80. The CAR of any one of claims 1 to 3, wherein the anti-ROR1 antibody or antigen binding fragment thereof comprises a variable light chain sequence as set forth in SEQ ID NO: 247 and/or a variable heavy chain sequence as set forth in SEQ ID NO: 248.

81. The CAR of any one of claims 1 to 3, wherein the anti-ROR1 antibody or antigen binding fragment thereof comprises a variable light chain sequence as set forth in SEQ ID NO: 255 and/or a variable heavy chain sequence as set forth in SEQ ID NO: 256.

82. The CAR of any one of claims 1 to 3, wherein the anti-ROR1 antibody or antigen binding fragment thereof comprises a variable light chain sequence as set forth in SEQ ID NO: 263 and/or a variable heavy chain sequence as set forth in SEQ ID NO: 264.

83. The CAR of any one of claims 1 to 3, wherein the anti-ROR1 antibody or antigen binding fragment thereof comprises a variable light chain sequence as set forth in SEQ ID NO: 271 and/or a variable heavy chain sequence as set forth in SEQ ID NO: 272.

84. The CAR of any one of claims 1 to 3, wherein the anti-ROR1 antibody or antigen binding fragment thereof comprises a variable light chain sequence as set forth in SEQ ID NO: 279 and/or a variable heavy chain sequence as set forth in SEQ ID NO: 280.

85. The CAR of any one of claims 1 to 3, wherein the anti-ROR1 antibody or antigen binding fragment thereof comprises a variable light chain sequence as set forth in SEQ ID NO: 287 and/or a variable heavy chain sequence as set forth in SEQ ID NO: 288.

86. The CAR of any one of claims 1 to 3, wherein the anti-ROR1 antibody or antigen binding fragment thereof comprises a variable light chain sequence as set forth in SEQ ID NO: 295 and/or a variable heavy chain sequence as set forth in SEQ ID NO: 296.

87. The CAR of any one of claims 1 to 3, wherein the anti-ROR1 antibody or antigen binding fragment thereof comprises a variable light chain sequence as set forth in SEQ ID NO: 303 and/or a variable heavy chain sequence as set forth in SEQ ID NO: 304.

88. The CAR of any one of claims 1 to 3, wherein the anti-ROR1 antibody or antigen binding fragment thereof comprises a variable light chain sequence as set forth in SEQ ID NO: 311 and/or a variable heavy chain sequence as set forth in SEQ ID NO: 312.

89. The CAR of any one of claims 1 to 3, wherein the anti-ROR1 antibody or antigen binding fragment thereof comprises a variable light chain sequence as set forth in SEQ ID NO: 319 and/or a variable heavy chain sequence as set forth in SEQ ID NO: 320.

90. The CAR of any one of claims 1 to 3, wherein the anti-ROR1 antibody or antigen binding fragment thereof comprises a variable light chain sequence as set forth in SEQ ID NO: 327 and/or a variable heavy chain sequence as set forth in SEQ ID NO: 328.

91. The CAR of any one of claims 1 to 3, wherein the anti-ROR1 antibody or antigen binding fragment thereof comprises a variable light chain sequence as set forth in SEQ ID NO: 335 and/or a variable heavy chain sequence as set forth in SEQ ID NO: 336.

92. The CAR of any one of claims 1 to 3, wherein the anti-ROR1 antibody or antigen binding fragment thereof comprises a variable light chain sequence as set forth in SEQ ID NO: 343 and/or a variable heavy chain sequence as set forth in SEQ ID NO: 344.

93. The CAR of any one of claims 1 to 3, wherein the anti-ROR1 antibody or antigen binding fragment thereof comprises a variable light chain sequence as set forth in SEQ ID NO: 351 and/or a variable heavy chain sequence as set forth in SEQ ID NO: 352.

94. The CAR of any one of claims 1 to 3, wherein the anti-ROR1 antibody or antigen binding fragment thereof comprises a variable light chain sequence as set forth in SEQ ID NO: 359 and/or a variable heavy chain sequence as set forth in SEQ ID NO: 360.

95. The CAR of any one of claims 1 to 94, wherein the transmembrane domain is from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, and PD1.

96. The CAR of any one of claims 1 to 95, wherein the transmembrane domain is from a polypeptide selected from the group consisting of: CD8α; CD4, CD45, PD1, and CD152.

97. The CAR of any one of claims 1 to 96, wherein the transmembrane domain is from CD8α.

98. The CAR of any one of claims 1 to 96, wherein the transmembrane domain is from PD1.

99. The CAR of any one of claims 1 to 96, wherein the transmembrane domain is from CD152.

100. The CAR of any one of claims 1 to 99, wherein the one or more co-stimulatory signaling domains are from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

101. The CAR of any one of claims 1 to 100, wherein the one or more co-stimulatory signaling domains are from a co-stimulatory molecule selected from the group consisting of: CD28, CD134, and CD137.

102. The CAR of any one of claims 1 to 101, wherein the one or more co-stimulatory signaling domains is from CD28.

103. The CAR of any one of claims 1 to 101, wherein the one or more co-stimulatory signaling domains is from CD134.

104. The CAR of any one of claims 1 to 101, wherein the one or more co-stimulatory signaling domains is from CD137.

105. The CAR of any one of claims 1 to 104, wherein the primary signaling domain isolated from a polypeptide selected from the group consisting of: FcRγ, Fc CD3γ, CD3δ, CD3ε, CD3ζ, CD22, CD79a, CD79b, and CD66d.

106. The CAR of any one of claims 1 to 105, wherein the primary signaling domain isolated from a CD3ζ.

107. The CAR of any one of claims 1 to 106, further comprising a hinge region polypeptide.

108. The CAR of claim 107, wherein the hinge region polypeptide comprises a hinge region of CD8α.

109. The CAR of claim 107, wherein the hinge region polypeptide comprises a hinge region of PD1.

110. The CAR of claim 107, wherein the hinge region polypeptide comprises a hinge region of CD152.

111. The CAR of any one of claims 1 to 110, further comprising a spacer region.

112. The CAR of claim 111, wherein the spacer region polypeptide comprises CH2 and CH3 regions of IgG1, IgG4, or IgD.

113. The CAR of any one of claims 1 to 112, further comprising a signal peptide.

114. The CAR of claim 113, wherein the signal peptide comprises an IgG1 heavy chain signal polypeptide, a CD8α signal polypeptide, or a human GM-CSF receptor alpha signal polypeptide.

115. The CAR of any one of claims 1 to 114, wherein the CAR comprises the amino acid sequence set forth in SEQ ID NOs: 386-397.

116. The CAR of any one of claims 1 to 114, wherein the CAR comprises the amino acid sequence set forth in SEQ ID NO: 386.

117. The CAR of any one of claims 1 to 114, wherein the CAR comprises the amino acid sequence set forth in SEQ ID NO: 387.

118. The CAR of any one of claims 1 to 114, wherein the CAR comprises the amino acid sequence set forth in SEQ ID NO: 388.

119. The CAR of any one of claims 1 to 114, wherein the CAR comprises the amino acid sequence set forth in SEQ ID NO: 389.

120. The CAR of any one of claims 1 to 114, wherein the CAR comprises the amino acid sequence set forth in SEQ ID NO: 390.

121. The CAR of any one of claims 1 to 114, wherein the CAR comprises the amino acid sequence set forth in SEQ ID NO: 391.

122. The CAR of any one of claims 1 to 114, wherein the CAR comprises the amino acid sequence set forth in SEQ ID NO: 392.

123. The CAR of any one of claims 1 to 114, wherein the CAR comprises the amino acid sequence set forth in SEQ ID NO: 393.

124. The CAR of any one of claims 1 to 114, wherein the CAR comprises the amino acid sequence set forth in SEQ ID NO: 394.

125. The CAR of any one of claims 1 to 114, wherein the CAR comprises the amino acid sequence set forth in SEQ ID NO: 395.

126. The CAR of any one of claims 1 to 114, wherein the CAR comprises the amino acid sequence set forth in SEQ ID NO: 396.

127. The CAR of any one of claims 1 to 114, wherein the CAR comprises the amino acid sequence set forth in SEQ ID NO: 397.

128. A polypeptide comprising the amino acid sequence of the CAR of any one of claims 1-127.

129. A polynucleotide encoding a CAR of any one of claims 1 to 127.

130. A vector comprising the polynucleotide of claim 129.

131. The vector of claim 130, wherein the vector is an expression vector.

132. The vector of claim 130 or claim 131, wherein the vector is an episomal vector.

133. The vector of any one of claims 130 to 132, wherein the vector is a viral vector.

134. The vector of any one of claims 130 to 133, wherein the vector is a retroviral vector.

135. The vector of any one of claims 130 to 134, wherein the vector is a lentiviral vector.

136. The vector of claim 135, wherein the lentiviral vector is selected from the group consisting essentially of: human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (Hy); bovine immune deficiency virus (BIV); and simian immunodeficiency virus (SIV).

137. The vector according to any one of claims 134 to 136, comprising a left (5′) retroviral LTR, a Psi (Ψ) packaging signal, a central polypurine tract/DNA flap (cPPT/FLAP), a retroviral export element; a promoter operably linked to the polynucleotide of claim 121; and a right (3′) retroviral LTR.

138. The vector of claim 137, further comprising a heterologous polyadenylation sequence.

139. The vector of claim 137 or claim 138, further comprising a hepatitis B virus post-transcriptional regulatory element (HPRE) or woodchuck post-transcriptional regulatory element (WPRE).

140. The vector of any one of claims 134 to 139, wherein the promoter of the 5′ LTR is replaced with a heterologous promoter.

141. The vector of claim 140, wherein the heterologous promoter is a cytomegalovirus (CMV) promoter, a Rous Sarcoma Virus (RSV)promoter, or an Simian Virus 40 (SV40) promoter.

142. The vector of any one of claims 137 to 141, wherein the 5′ LTR or 3′ LTR is a lentivirus LTR.

143. The vector of any one of claims 137 to 141, wherein the 3′ LTR comprises one or more modifications.

144. The vector of any one of claims 137 to 143, wherein the 3′ LTR comprises one or more deletions.

145. The vector of any one of claims 137 to 144, wherein the 3′ LTR is a self-inactivating (SIN) LTR.

146. The vector of any one of claims 138 to 145, wherein the polyadenylation sequence is a bovine growth hormone polyadenylation or signal rabbit β-globin polyadenylation sequence.

147. The vector of any one of claims 140 to 145, wherein the polynucleotide of claim 64 comprises an optimized Kozak sequence.

148. The vector of any one of claims 137 to 147, wherein the promoter operably linked to the polynucleotide of claim 129 is selected from the group consisting of: a cytomegalovirus immediate early gene promoter (CMV), an elongation factor 1 alpha promoter (EF1-α), a phosphoglycerate kinase-1 promoter (PGK), a ubiquitin-C promoter (UBQ-C), a cytomegalovirus enhancer/chicken beta-actin promoter (CAG), polyoma enhancer/herpes simplex thymidine kinase promoter (MC1), a beta actin promoter (β-ACT), a simian virus 40 promoter (SV40), and a myeloproliferative sarcoma virus enhancer, negative control region deleted, d1587rev primer-binding site substituted (MND) promoter.

149. An immune effector cell comprising the vector of any one of claims 130 to 148.

150. The immune effector cell of claim 149, wherein the immune effector cell is selected from the group consisting of: a T lymphocyte and a natural killer (NK) cell.

151. The immune effector cell of claim 149 or 150, wherein the immune effector cell is transduced with the vector of any one of claims 130 to 148 and is activated and stimulated in the presence of an inhibitor of the PI3K pathway, thereby maintaining proliferation of the transduced immune effector cells compared to the proliferation of transduced immune effector cells that were activated and stimulated in the absence of the inhibitor of the PI3K pathway.

152. The immune effector cell of claim 151, wherein the immune effector cell activated and stimulated in the presence of the inhibitor of PI3K pathway has increased expression of i) one or more markers selected from the group consisting of: CD62L, CD127, CD197, and CD38 or ii) all of the markers CD62L, CD127, CD197, and CD38 compared to an immune effector cell activated and stimulated in the absence of the inhibitor of PI3K pathway.

153. The immune effector cell of claim 151, wherein the immune effector cell activated and stimulated in the presence of the inhibitor of PI3K pathway has increased expression of i) one or more markers selected from the group consisting of: CD62L, CD127, CD27, and CD8 or ii) all of the markers CD62L, CD127, CD27, and CD8 compared to an immune effector cell activated and stimulated in the absence of the inhibitor of PI3K pathway.

154. The immune effector cell of any one of claims 151-153, wherein the PI3K inhibitor is ZSTK474.

155. A composition comprising the immune effector cell of any one of claims 149-154 and a physiologically acceptable excipient.

156. A method of generating an immune effector cell comprising a CAR according to any one of claims 1 to 127 comprising introducing into an immune effector cell the vector of any one of claims 130 to 148.

157. The method of claim 156, further comprising stimulating the immune effector cell and inducing the cell to proliferate by contacting the cell with antibodies that bind CD3 and antibodies that bind to CD28; thereby generating a population of immune effector cells.

158. The method of claim 157, wherein the immune effector cell is stimulated and induced to proliferate before introducing the vector.

159. The method of claim 158, wherein the immune effector cells comprise T lymphocytes.

160. The method of claim 158, wherein the immune effector cells comprise NK cells.

161. The method of any one of claims 156 to 160, wherein the immune effector cell is activated and stimulated in the presence of the inhibitor of PI3K pathway has increased expression of i) one or more markers selected from the group consisting of: CD62L, CD127, CD197, and CD38 or ii) all of the markers CD62L, CD127, CD197, and CD38 compared to an immune effector cell activated and stimulated in the absence of the inhibitor of PI3K pathway.

162. The method of any one of claims 156 to 160, wherein the immune effector cell is activated and stimulated in the presence of the inhibitor of PI3K pathway has increased expression of i) one or more markers selected from the group consisting of: CD62L, CD127, CD27, and CD8 or ii) all of the markers CD62L, CD127, CD27, and CD8 compared to an immune effector cell activated and stimulated in the absence of the inhibitor of PI3K pathway.

163. The method of any one of claims 156 to 162, wherein the PI3K inhibitor is ZSTK474.

164. A method for increasing the cytotoxicity in cancer cells that express ROR1 in a subject, comprising administering to the subject an amount of the composition of claim 155 sufficient to increase the cytotoxicity in cancer cells that express ROR1 compared to the cytotoxicity of the cancer cells that express ROR1 prior to the administration.

165. A method for decreasing the number of cancer cells expressing ROR1 in a subject, comprising administering to the subject an amount of the composition of claim 155 sufficient to decrease the number of cancer cells that express ROR1 compared to the number of the cancer cells that express ROR1 prior to the administration.

166. A method of treating a cancer in a subject in need thereof, comprising administering to the subject a therapeutically effect amount of the composition of claim 155.

167. The method of any one of claims 164 to 167, wherein the cancer is a solid cancer.

168. The method of any one of claims 164 to 167, wherein the cancer is a liquid cancer.

169. The method of any one of claims 164 to 167, wherein the cancer is a hematological malignancy.

170. The method of any one of claims 164 to 167, wherein the cancer is lung cancer, breast cancer, pancreatic cancer, ovarian cancer, prostate cancer, adrenal cancer, melanoma, uterine cancer, testicular cancer, bladder cancer, non-Hodgkin's lymphoma, acute lymphocytic leukemia (ALL), chronic lymphocytic leukemia (CLL), hairy cell leukemia (HCL), multiple myeloma (MM), acute myeloid leukemia (AML), or chronic myeloid leukemia (CML).

171. The method of any one of claims 164 to 167, wherein the cancer is lung cancer, breast cancer, pancreatic cancer, ovarian cancer, prostate cancer, adrenal cancer, melanoma, uterine cancer, testicular cancer, or bladder cancer.

172. The method of any one of claims 164 to 167, wherein the cancer is small lymphocytic lymphoma (SLL), diffuse large B cell lymphoma (DLBCL), follicular lymphoma (FL), mantle cell lymphoma (MCL), or marginal zone lymphoma (MZL).

173. The method of any one of claims 164 to 167, wherein the cancer is acute lymphocytic leukemia (ALL), chronic lymphocytic leukemia (CLL), hairy cell leukemia (HCL), multiple myeloma (MM), acute myeloid leukemia (AML), or chronic myeloid leukemia (CML).

174. A method for ameliorating at one or more symptoms associated with a cancer expressing ROR1 in a subject, comprising administering to the subject an amount of the composition of claim 155 sufficient to ameliorate at least one symptom associated with cancer cells that express ROR1.

175. The method of claim 174, wherein the one or more symptoms ameliorated are selected from the group consisting of: weakness, fatigue, shortness of breath, easy bruising and bleeding, frequent infections, enlarged lymph nodes, distended or painful abdomen, bone or joint pain, fractures, unplanned weight loss, poor appetite, night sweats, persistent mild fever, and decreased urination.


SEQUENCE LISTING (402 sequences)

SEQ ID NO: 1 - protein, 11 aa, Homo sapiens
QASQDISNYLN

SEQ ID NO: 2 - protein, 7 aa, Homo sapiens
DASNLET

SEQ ID NO: 3 - protein, 9 aa, Homo sapiens
QQYDNLPLT

SEQ ID NO: 4 - protein, 10 aa, Homo sapiens
GFTFSDYYMS

SEQ ID NO: 5 - protein, 17 aa, Homo sapiens
YISDSTNTIYYADSVKG

SEQ ID NO: 6 - protein, 10 aa, Homo sapiens
AVGAGEGFDH

SEQ ID NO: 7 - protein, 108 aa, Homo sapiens
AIRMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIKR

SEQ ID NO: 8 - protein, 119 aa, Homo sapiens
EVQLVESGGGLVKPGGSLRLSCAASGFTFSDYYMSWIRQAPGKGLEWVSYISDSTNTIYYADSVKGRFTVSRDNPKNSLYLQMISLRAEDTAVYYCARAVGAGEGFDHWGQGTLVTVSS

SEQ ID NO: 9 - protein, 11 aa, Homo sapiens
QASQDISNYLN

SEQ ID NO: 10 - protein, 7 aa, Homo sapiens
DASNLET

SEQ ID NO: 11 - protein, 9 aa, Homo sapiens
QQYDNLPLT

SEQ ID NO: 12 - protein, 10 aa, Homo sapiens
GFTFSDYYMG

SEQ ID NO: 13 - protein, 17 aa, Homo sapiens
YISDRAHTIYDTDSVKG

SEQ ID NO: 14 - protein, 10 aa, Homo sapiens
AVGAGEGFDY

SEQ ID NO: 15 - protein, 108 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKLEIKR

SEQ ID NO: 16 - protein, 119 aa, Homo sapiens
EVQLVESGGGLVKPGGSLRLSCAASGFTFSDYYMGWVRQAPGKGLKWLSYISDRAHTIYDTDSVKGRFTISRDDAKSSLYLRMNNLRVEDTAVYYCARAVGAGEGFDYWGQGTLVTVSS

SEQ ID NO: 17 - protein, 11 aa, Homo sapiens
QASQDISNYLN

SEQ ID NO: 18 - protein, 7 aa, Homo sapiens
AASSLQS

SEQ ID NO: 19 - protein, 9 aa, Homo sapiens
QQSYSTPFT

SEQ ID NO: 20 - protein, 10 aa, Homo sapiens
GYTFNNYGFS

SEQ ID NO: 21 - protein, 17 aa, Homo sapiens
WISVYNGNTNYAQKLQG

SEQ ID NO: 22 - protein, 16 aa, Homo sapiens
DYYSDSSGYWDDAFDI

SEQ ID NO: 23 - protein, 108 aa, Homo sapiens
AIRMTQSPSSLSASVGDRVTITCQASQDISNYLNWYQQKPGKAPKLLIYAASSLQSGVPSRFSGSGSGTDFTLTISSLQPEDFATYYCQQSYSTPFTFGPGTKVDIKR

SEQ ID NO: 24 - protein, 125 aa, Homo sapiens
EVQLVQSGAEVKKPGASVKVSCKASGYTFNNYGFSWVRQAPGQGLEWMGWISVYNGNTNYAQKLQGRVTMTTDTSTSTAYMELRSLRSDDTAVYYCARDYYSDSSGYWDDAFDIWGQGTMVTVSS

SEQ ID NO: 25 - protein, 11 aa, Homo sapiens
RASQGISSWLA

SEQ ID NO: 26 - protein, 7 aa, Homo sapiens
AASSLQS

SEQ ID NO: 27 - protein, 9 aa, Homo sapiens
QQANSFPLT

SEQ ID NO: 28 - protein, 10 aa, Homo sapiens
GYSFSRYWIG

SEQ ID NO: 29 - protein, 17 aa, Homo sapiens
IIYPRDSDTRYSPSFQG

SEQ ID NO: 30 - protein, 10 aa, Homo sapiens
PVVTAGAFDI

SEQ ID NO: 31 - protein, 108 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKR

SEQ ID NO: 32 - protein, 119 aa, Homo sapiens
EVQLVQSGAEVKKPGESLKISCKGSGYSFSRYWIGWVRQMPGKGLEWMGIIYPRDSDTRYSPSFQGQVTISADKSISTAYLQWSSLKASDTAMYYCATPVVTAGAFDIWGQGTMVTVSS

SEQ ID NO: 33 - protein, 11 aa, Homo sapiens
RASQSISSHLN

SEQ ID NO: 34 - protein, 7 aa, Homo sapiens
AASSLQS

SEQ ID NO: 35 - protein, 9 aa, Homo sapiens
QQSYSTPFT

SEQ ID NO: 36 - protein, 10 aa, Homo sapiens
GHTFSNYGIS

SEQ ID NO: 37 - protein, 17 aa, Homo sapiens
WISTYNGNTNYAQKLQG

SEQ ID NO: 38 - protein, 16 aa, Homo sapiens
DYYSDSSGYWDDAFDI

SEQ ID NO: 39 - protein, 108 aa, Homo sapiens
AIQMTQSPSSLSASVGDRVTITCRASQSISSHLNWYQQKPGKAPKLLIYAASSLQSGVPSRFSGSGSGTDFTLTISSLQPEDFATYYCQQSYSTPFTFGPGTKVDIKR

SEQ ID NO: 40 - protein, 125 aa, Homo sapiens
QVQLQQSGAEVKKPGASVKVSCKASGHTFSNYGISWVRQAPGQGLEWMGWISTYNGNTNYAQKLQGRVTMTTDTSTSTAYMELRSLRSDDTAVYYCARDYYSDSSGYWDDAFDIWGQGTTVTVSS

SEQ ID NO: 41 - protein, 11 aa, Homo sapiens
QASQDVRNYLN

SEQ ID NO: 42 - protein, 7 aa, Homo sapiens
DATNLES

SEQ ID NO: 43 - protein, 9 aa, Homo sapiens
QQYDNLPLS

SEQ ID NO: 44 - protein, 10 aa, Homo sapiens
GFTFSDYYMG

SEQ ID NO: 45 - protein, 17 aa, Homo sapiens
YISDRAHTIYDTDSVKG

SEQ ID NO: 46 - protein, 10 aa, Homo sapiens
AVGAGEGFDY

SEQ ID NO: 47 - protein, 108 aa, Homo sapiens
DIQLTQSPSSLSASVGDRVTITCQASQDVRNYLNWYQQKPGKAPNLLIYDATNLESGVPSRFSGSGSGTDFTFTISSLQPEDFATYYCQQYDNLPLSFGGGTKVEIKR

SEQ ID NO: 48 - protein, 119 aa, Homo sapiens
EAQLVESGGGLVKPGGSLRLSCAASGFTFSDYYMGWVRQAPGKGLKWLSYISDRAHTIYDTDSVKGRFTISRDDAKSSLYLRMNNLRVEDTAVYYCARAVGAGEGFDYWGQGTLVTVSS

SEQ ID NO: 49 - protein, 11 aa, Homo sapiens
RASQSVSSNLA

SEQ ID NO: 50 - protein, 7 aa, Homo sapiens
GASTRAT

SEQ ID NO: 51 - protein, 10 aa, Homo sapiens
QQYNNWPPYT

SEQ ID NO: 52 - protein, 12 aa, Homo sapiens
GFSLSSFGVAVG

SEQ ID NO: 53 - protein, 16 aa, Homo sapiens
LIYWDDDKRYSPSLKT

SEQ ID NO: 54 - protein, 15 aa, Homo sapiens
KGGIATTGSPNWFDP

SEQ ID NO: 55 - protein, 109 aa, Homo sapiens
EIVLTQSPDTLSLSPGERATLSCRASQSVSSNLAWYQQKPGQAPRLLIYGASTRATGIPARFSGSGSGTEFTLTISSLQSEDFAVYYCQQYNNWPPYTFGQGTKVEIKR

SEQ ID NO: 56 - protein, 125 aa, Homo sapiens
QVTLKESGPTLVKPTQTLTLTCTFSGFSLSSFGVAVGWFRQPPGKALEWLGLIYWDDDKRYSPSLKTRLTITKDTSKNQVVLTMTNMDPVDTATYYCAHKGGIATTGSPNWFDPWGQGTLVTVSS

SEQ ID NO: 57 - protein, 11 aa, Homo sapiens
RASQSVSSNLA

SEQ ID NO: 58 - protein, 7 aa, Homo sapiens
GASTRAT

SEQ ID NO: 59 - protein, 10 aa, Homo sapiens
QQYNNWPPYT

SEQ ID NO: 60 - protein, 12 aa, Homo sapiens
GFSLNSFGVAVG

SEQ ID NO: 61 - protein, 16 aa, Homo sapiens
LIYWDDDRRYFPSLEG

SEQ ID NO: 62 - protein, 16 aa, Homo sapiens
TSPMVQGIANYYAMDV

SEQ ID NO: 63 - protein, 109 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPPYTFGQGTKVEIKR

SEQ ID NO: 64 - protein, 126 aa, Homo sapiens
QVTLKESGPTLVKPTQTLTLTCTFSGFSLNSFGVAVGWFRQPPGKALEWLGLIYWDDDRRYFPSLEGRLSITKDASDNNVVLTMMNVDPADTATYYCARTSPMVQGIANYYAMDVWGQGTTVTVSS

SEQ ID NO: 65 - protein, 11 aa, Homo sapiens
GGTNIGSESVH

SEQ ID NO: 66 - protein, 7 aa, Homo sapiens
DDTDRPS

SEQ ID NO: 67 - protein, 11 aa, Homo sapiens
QVWDSVSDRYV

SEQ ID NO: 68 - protein, 12 aa, Homo sapiens
GGSISRSDGYWG

SEQ ID NO: 69 - protein, 16 aa, Homo sapiens
SIYDTGTTYYSPSLKS

SEQ ID NO: 70 - protein, 14 aa, Homo sapiens
MGGLRSSSSDAFHT

SEQ ID NO: 71 - protein, 109 aa, Homo sapiens
QSVLTQPPSVSVAPGETARITCGGTNIGSESVHWYQQRPGQAPVLVVYDDTDRPSGIPERFSGSNSGNTATLTISRVEAGDGADYYCQVWDSVSDRYVFGTGTKVTVLG

SEQ ID NO: 72 - protein, 124 aa, Homo sapiens
QLQLQESGPGVVKPSGTLSLTCTVSGGSISRSDGYWGWVRQPPGKGLEWIGSIYDTGTTYYSPSLKSRLIISVDTSKNQFSLTLNSVTAADTAVYYCASMGGLRSSSSDAFHTWGPGTMVTVSS

SEQ ID NO: 73 - protein, 14 aa, Homo sapiens
TGTSSDVGGYNYVS

SEQ ID NO: 74 - protein, 7 aa, Homo sapiens
DVSNRPS

SEQ ID NO: 75 - protein, 11 aa, Homo sapiens
SSYTSSSTLWV

SEQ ID NO: 76 - protein, 10 aa, Homo sapiens
GYSFTNYWLG

SEQ ID NO: 77 - protein, 17 aa, Homo sapiens
IIYPGDSDTRYSPSFRG

SEQ ID NO: 78 - protein, 11 aa, Homo sapiens
LNLATHTAFDI

SEQ ID NO: 79 - protein, 112 aa, Homo sapiens
QSVLTQPASVSGSPGQSITISCTGTSSDVGGYNYVSWYQQHPGKAPKLMIYDVSNRPSGVSNRFSGSKSGNTASLTISGLQAEDEADYYCSSYTSSSTLWVFGGGTKLTVLG

SEQ ID NO: 80 - protein, 120 aa, Homo sapiens
QVQLVQSGAEVKKPGESLKISCKDSGYSFTNYWLGWVRQMPGKGLEWMGIIYPGDSDTRYSPSFRGQVTISADKSISTAYLQWSSLKASDTAMYYCARLNLATHTAFDIWGQGTTVTVSS

SEQ ID NO: 81 - protein, 14 aa, Homo sapiens
TGTSSDVGGYNYVS

SEQ ID NO: 82 - protein, 7 aa, Homo sapiens
DVSKRPS

SEQ ID NO: 83 - protein, 10 aa, Homo sapiens
GSFTSSITYV

SEQ ID NO: 84 - protein, 10 aa, Homo sapiens
GYTFTSYYMH

SEQ ID NO: 85 - protein, 17 aa, Homo sapiens
IINPSGGSTSYAQKFQG

SEQ ID NO: 86 - protein, 11 aa, Homo sapiens
GGYTGWSPSDP

SEQ ID NO: 87 - protein, 111 aa, Homo sapiens
QSALTQPASVSGSPGQSITISCTGTSSDVGGYNYVSWYQQHPGKAPKFMIYDVSKRPSGVSNRFSGSKSGNTASLTISGLQAEDEADYYCGSFTSSITYVFGTGTKVTVLG

SEQ ID NO: 88 - protein, 120 aa, Homo sapiens
QMQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGYTGWSPSDPWGQGTLVTVSS

SEQ ID NO: 89 - protein, 14 aa, Homo sapiens
TGTSSDVGGYNYVS

SEQ ID NO: 90 - protein, 7 aa, Homo sapiens
DVSNRPS

SEQ ID NO: 91 - protein, 9 aa, Homo sapiens
SSYTSSSTL

SEQ ID NO: 92 - protein, 10 aa, Homo sapiens
GFTFSSYSMN

SEQ ID NO: 93 - protein, 17 aa, Homo sapiens
SISSSSSYIYYADSVKG

SEQ ID NO: 94 - protein, 12 aa, Homo sapiens
GLGGWTHDAFDI

SEQ ID NO: 95 - protein, 110 aa, Homo sapiens
QSALTQPASVSGSPGQSITISCTGTSSDVGGYNYVSWYQQHPGKAPKLMIFDVSNRPSGVSNRFSGSKSGNTASLTISGLQAEDEADYYCSSYTSSSTLFGGGTKLTVLG

SEQ ID NO: 96 - protein, 121 aa, Homo sapiens
EVQLVQSGGDLVQPGGSLRLSCAASGFTFSSYSMNWVRQAPGKGLEWVSSISSSSSYIYYADSVKGRFTISRDNAKNSLYLQMNSLRAEDTAVYYCARGLGGWTHDAFDIWGQGTTVTVSS

SEQ ID NO: 97 - protein, 13 aa, Homo sapiens
PGSSSNIGSNYVY

SEQ ID NO: 98 - protein, 7 aa, Homo sapiens
RNNQRPS

SEQ ID NO: 99 - protein, 11 aa, Homo sapiens
GTWDSSLSAYV

SEQ ID NO: 100 - protein, 10 aa, Homo sapiens
GYTFSRYYIH

SEQ ID NO: 101 - protein, 17 aa, Homo sapiens
LINPGGGSTNYAQKFQG

SEQ ID NO: 102 - protein, 12 aa, Homo sapiens
DYGTIDARRFDF

SEQ ID NO: 103 - protein, 111 aa, Homo sapiens
QSALTQPPSASGTPGQRVTISCPGSSSNIGSNYVYWYQQLPGTAPKLLIYRNNQRPSGVPDRFSGSKSGTSATLGITGLQTGDEADYYCGTWDSSLSAYVFGTGTKVTVLG

SEQ ID NO: 104 - protein, 121 aa, Homo sapiens
QVQLQQSGAEVKKPGASVKVSCKASGYTFSRYYIHWVRRAPGQGLEWMGLINPGGGSTNYAQKFQGRVTMTRDTSTNTVYLELSSLRSDDTAVYYCARDYGTIDARRFDFWGQGTLVTVSS

SEQ ID NO: 105 - protein, 14 aa, Homo sapiens
TGTSSDVGGYNYVS

SEQ ID NO: 106 - protein, 7 aa, Homo sapiens
DVSNRPS

SEQ ID NO: 107 - protein, 11 aa, Homo sapiens
SSYTSSSIPWV

SEQ ID NO: 108 - protein, 10 aa, Homo sapiens
GYSFTSYWIG

SEQ ID NO: 109 - protein, 17 aa, Homo sapiens
IIYPGDSDTRYSPSFQG

SEQ ID NO: 110 - protein, 11 aa, Homo sapiens
LSSSSYDAFDI

SEQ ID NO: 111 - protein, 112 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSIPWVFGGGTKLTVLG

SEQ ID NO: 112 - protein, 120 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSSSSYDAFDIWGQGTMVTVSS

SEQ ID NO: 113 - protein, 14 aa, Homo sapiens
TGTSSDFGDYDYVS

SEQ ID NO: 114 - protein, 7 aa, Homo sapiens
DVSDRPS

SEQ ID NO: 115 - protein, 10 aa, Homo sapiens
SSFTTSSTLV

SEQ ID NO: 116 - protein, 10 aa, Homo sapiens
GGSFKTHGIS

SEQ ID NO: 117 - protein, 17 aa, Homo sapiens
WINPNSGGALYVDNFQG

SEQ ID NO: 118 - protein, 11 aa, Homo sapiens
GMADLIDVFDI

SEQ ID NO: 119 - protein, 116 aa, Homo sapiens
QSALTQPALTQPASVSGSPGQSITISCTGTSSDFGDYDYVSWYQQHPGKAPKLMIYDVSDRPSGVSNRFSGSKSGNTASLTISGLQAEDEADYFCSSFTTSSTLVFGGGTKLTVLG

SEQ ID NO: 120 - protein, 120 aa, Homo sapiens
EVQLVQSGAELKKPGSSVRVSCKTSGGSFKTHGISWVRQAPGQGLEWMGWINPNSGGALYVDNFQGRATMTRDTSINTAYMELRSLSSDDTAVYYCARGMADLIDVFDIWGQGTMVTVSS

SEQ ID NO: 121 - protein, 14 aa, Homo sapiens
TGTSRDVGGYDYVS

SEQ ID NO: 122 - protein, 7 aa, Homo sapiens
DVSRRPS

SEQ ID NO: 123 - protein, 10 aa, Homo sapiens
SSYTSSSTRV

SEQ ID NO: 124 - protein, 10 aa, Homo sapiens
GFTFSSYSMN

SEQ ID NO: 125 - protein, 17 aa, Homo sapiens
SISSSSSYIYYADSVKG

SEQ ID NO: 126 - protein, 12 aa, Homo sapiens
GLGGWTHDAFDI

SEQ ID NO: 127 - protein, 111 aa, Homo sapiens
QSALTQPASVSGSPGQSITISCTGTSRDVGGYDYVSWYQQYPGNAPKLMIYDVSRRPSGVSHRFSASKSGNTASLTISGLQTEDEADYYCSSYTSSSTRVFGGGTKVTVLG

SEQ ID NO: 128 - protein, 121 aa, Homo sapiens
EVQLVQSGGGVVQPGGPLRLSCAASGFTFSSYSMNWVRQAPGKGLEWVSSISSSSSYIYYADSVKGRFTISRDNAKNSLYLQMNSLRAEDTAVYYCARGLGGWTHDAFDIWGQGTMVTVSS

SEQ ID NO: 129 - protein, 14 aa, Homo sapiens
TGTSSDVGGYNYVS

SEQ ID NO: 130 - protein, 7 aa, Homo sapiens
DVSKRPS

SEQ ID NO: 131 - protein, 11 aa, Homo sapiens
SSYTSSSTSVV

SEQ ID NO: 132 - protein, 10 aa, Homo sapiens
GFTFSSYSMN

SEQ ID NO: 133 - protein, 17 aa, Homo sapiens
SISSSSSYIYYADSVKG

SEQ ID NO: 134 - protein, 12 aa, Homo sapiens
GLGGWTHDAFDI

SEQ ID NO: 135 - protein, 112 aa, Homo sapiens
QSALTQPASVSGSPGQSITISCTGTSSDVGGYNYVSWYQQHPGKAPKLMIYDVSKRPSGVSNRFSGSKSGNTASLTISGLQAEDEADYYCSSYTSSSTSVVFGGGTKLTVLG

SEQ ID NO: 136 - protein, 121 aa, Homo sapiens
QMQLVQSGGDLVQPGGSLRLSCAASGFTFSSYSMNWVRQAPGKGLEWVSSISSSSSYIYYADSVKGRFTISRDNAKNSLYLQMNSLRAEDTAVYYCARGLGGWTHDAFDIWGQGTTVTVSS

SEQ ID NO: 137 - protein, 14 aa, Homo sapiens
TGTTSDVGGYNYVS

SEQ ID NO: 138 - protein, 7 aa, Homo sapiens
DVSKRPS

SEQ ID NO: 139 - protein, 10 aa, Homo sapiens
SSYTSSSTDV

SEQ ID NO: 140 - protein, 10 aa, Homo sapiens
GFTFSSYSMN

SEQ ID NO: 141 - protein, 17 aa, Homo sapiens
SISSSSSYIYYADSVKG

SEQ ID NO: 142 - protein, 12 aa, Homo sapiens
GLGGWTHDAFDI

SEQ ID NO: 143 - protein, 111 aa, Homo sapiens
QSVLTQPASVSGSPGQSITISCTGTTSDVGGYNYVSWYQQHPGKAPKLMIYDVSKRPSGVSNRFSGSKSGNTASLTISGLQAEDEADYYCSSYTSSSTDVFGTGTKLTVLG

SEQ ID NO: 144 - protein, 121 aa, Homo sapiens; identical to SEQ ID NO: 96

SEQ ID NO: 145 - protein, 14 aa, Homo sapiens
TGTSSDVGGYNYVT

SEQ ID NO: 146 - protein, 7 aa, Homo sapiens
DVSKRPS

SEQ ID NO: 147 - protein, 10 aa, Homo sapiens
SSYTSSSTLV

SEQ ID NO: 148 - protein, 10 aa, Homo sapiens
GFTFGTYSMN

SEQ ID NO: 149 - protein, 17 aa, Homo sapiens
SISSSSSYIYYADSVKG

SEQ ID NO: 150 - protein, 12 aa, Homo sapiens
GLGGWTHDAFDI

SEQ ID NO: 151 - protein, 111 aa, Homo sapiens
QSALTQPASVSASPGQSITISCTGTSSDVGGYNYVTWYQQHPGKAPKLMIYDVSKRPSGVLDRFSGSKSGNTASLTISGLQAEDEADYFCSSYTSSSTLVFGGGTKLTVLG

SEQ ID NO: 152 - protein, 121 aa, Homo sapiens
QVQLVQSGGGLVKPGGSLRLSCAASGFTFGTYSMNWVRQAPGKGLEWVSSISSSSSYIYYADSVKGRFTISRDNAKNSLYLQMNSLRAEDTAVYYCARGLGGWTHDAFDIWGQGTTVTVSS

SEQ ID NO: 153 - protein, 13 aa, Homo sapiens
SGSSSNIGSNYVY

SEQ ID NO: 154 - protein, 7 aa, Homo sapiens
RNNQRPS

SEQ ID NO: 155 - protein, 11 aa, Homo sapiens
AAWDDSLSGYV

SEQ ID NO: 156 - protein, 10 aa, Homo sapiens
GYTFSRYYIH

SEQ ID NO: 157 - protein, 17 aa, Homo sapiens
LINPGGGSTNYAQKFQG

SEQ ID NO: 158 - protein, 12 aa, Homo sapiens
DYGTIDARRFDF

SEQ ID NO: 159 - protein, 111 aa, Homo sapiens
QSALTQPPSASGTPGQRVTISCSGSSSNIGSNYVYWYQQLPGTAPKLLIYRNNQRPSGVPDRFSGSKSGTSASLAISGLRSEDEADYYCAAWDDSLSGYVFGTGTKVTVLG

SEQ ID NO: 160 - protein, 121 aa, Homo sapiens
QMQLVQSGAEVKKPGASVKVSCKASGYTFSRYYIHWVRQAPGQGLEWMGLINPGGGSTNYAQKFQGRVTMTRDTSTNTVYLELSSLRSDDTAVYYCARDYGTIDARRFDFWGQGTLVTVSS

SEQ ID NO: 161 - protein, 13 aa, Homo sapiens
SGGISNVGTNGVN

SEQ ID NO: 162 - protein, 7 aa, Homo sapiens
AMNQRPS

SEQ ID NO: 163 - protein, 11 aa, Homo sapiens
ATWDDSLSGVL

SEQ ID NO: 164 - protein, 11 aa, Homo sapiens
GGSISSSNWWS

SEQ ID NO: 165 - protein, 16 aa, Homo sapiens
EIYHSGSTNYNPSLKS

SEQ ID NO: 166 - protein, 9 aa, Homo sapiens
DLWLGEWDL

SEQ ID NO: 167 - protein, 111 aa, Homo sapiens
QSVLTQPPSASGAPGQRVTISCSGGISNVGTNGVNWYQHLPGTAPKLLVDAMNQRPSGVPDRFSGSRSGTSGSLAITGLRSEDEADYYCATWDDSLSGVLFGGGTKLTVLG

SEQ ID NO: 168 - protein, 118 aa, Homo sapiens
EVQLVQSGPGLVKPSGTLSLTCAVSGGSISSSNWWSWVRQPPGKGLEWIGEIYHSGSTNYNPSLKSRVTISVDKSKNQFSLKLGSVTAADTATYYCARDLWLGEWDLWGQGTLVTVSS

SEQ ID NO: 169 - protein, 11 aa, Homo sapiens
QASQDIRNYLN

SEQ ID NO: 170 - protein, 7 aa, Homo sapiens
AASNLET

SEQ ID NO: 171 - protein, 9 aa, Homo sapiens
QQDDNLPLT

SEQ ID NO: 172 - protein, 10 aa, Homo sapiens
GFTFSDYYMG

SEQ ID NO: 173 - protein, 17 aa, Homo sapiens
YISDRAHTIYDTHSVKG

SEQ ID NO: 174 - protein, 10 aa, Homo sapiens
AVGAGEGFDY

SEQ ID NO: 175 - protein, 108 aa, Homo sapiens
DIQMTQSPSSLSASVGDRVTITCQASQDIRNYLNWYQQKPGKAPKLLIYAASNLETGVPSRFSGSGSGTDFTFTISSLQPEDIATYYCQQDDNLPLTFGGGTKLEIKR

SEQ ID NO: 176 - protein, 119 aa, Homo sapiens
EVQLVESGGGLVKPGGSLKLSCAASGFTFSDYYMGWVRQAPGKGLKWLSYISDRAHTIYDTHSVKGRFTISRDDAKSSLYLRMNNLRVEDTAVYYCARAVGAGEGFDYWCQGTLVTVSS

SEQ ID NO: 177 - protein, 11 aa, Homo sapiens
QASQDISNYLN

SEQ ID NO: 178 - protein, 7 aa, Homo sapiens
DASNLET

SEQ ID NO: 179 - protein, 9 aa, Homo sapiens
QQFDNLPYT

SEQ ID NO: 180 - protein, 10 aa, Homo sapiens
GGTFSTFAIN

SEQ ID NO: 181 - protein, 17 aa, Homo sapiens
GVIPVSGTEDYSQKFQG

SEQ ID NO: 182 - protein, 12 aa, Homo sapiens
DRSGRDWDYFDY

SEQ ID NO: 183 - protein, 108 aa, Homo sapiens
DIVMTQSPSSLSASVGDRVTITCQASQDISNYLNWYQQKPGKAPKLLIYDASNLETGVPSRFSGSGSGTDFIFTISSLQPEDIATYYCQQFDNLPYTFGQGTKVEIKR

SEQ ID NO: 184 - protein, 121 aa, Homo sapiens
EVQLVQSGAEVKKPGSSVKVSCKASGGTFSTFAINWVRQAPGQGLEWMGGVIPVSGTEDYSQKFQGRLSLTADESTGTAYMELSSLRSDDTAVYYCARDRSGRDWDYFDYWGQGTLVTVSS

SEQ ID NO: 185 - protein, 12 aa, Homo sapiens
RASQSVSSSYLA

SEQ ID NO: 186 - protein, 7 aa, Homo sapiens
GASSRAT

SEQ ID NO: 187 - protein, 6 aa, Homo sapiens
QQYGSL

SEQ ID NO: 188 - protein, 10 aa, Homo sapiens
GGSLSSHGVS

SEQ ID NO: 189 - protein, 17 aa, Homo sapiens
RIIPMFGLTDYAQNFQA

SEQ ID NO: 190 - protein, 9 aa, Homo sapiens
ESLGATFEY

SEQ ID NO: 191 - protein, 106 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSLFGQGTKVEIKR

SEQ ID NO: 192 - protein, 118 aa, Homo sapiens
EVQLVQSGAEVRKPGSSVKVSCKASGGSLSSHGVSWVRQAPGQGLEWMARIIPMFGLTDYAQNFQARVTISADRSTNTVYMEISNLGSEDTAVYFCARESLGATFEYWGQGTLVTVSS

SEQ ID NO: 193 - protein, 12 aa, Homo sapiens
RASQSVSSSYLA

SEQ ID NO: 194 - protein, 7 aa, Homo sapiens
GASSRAT

SEQ ID NO: 195 - protein, 6 aa, Homo sapiens
QQYGSS

SEQ ID NO: 196 - protein, 10 aa, Homo sapiens
GGSLSSHGVS

SEQ ID NO: 197 - protein, 17 aa, Homo sapiens
RIIPMFGVTDYAQKFQD

SEQ ID NO: 198 - protein, 9 aa, Homo sapiens
ESRGATFEY

SEQ ID NO: 199 - protein, 106 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSFGPGTKVDIKR

SEQ ID NO: 200 - protein, 118 aa, Homo sapiens
QVQLVQSGTEVKKPGSSVKVSCQASGGSLSSHGVSWLRQAPGQGLEWVGRIIPMFGVTDYAQKFQDRVTITADKSTSTVYMELISLGSDDTAVYFCARESRGATFEYWGQGTLVTVSS

SEQ ID NO: 201 - protein, 11 aa, Homo sapiens
RASQNINNYLN

SEQ ID NO: 202 - protein, 7 aa, Homo sapiens
AASSLQS

SEQ ID NO: 203 - protein, 9 aa, Homo sapiens
QQSYNTPFT

SEQ ID NO: 204 - protein, 10 aa, Homo sapiens
GYTSTNYGIS

SEQ ID NO: 205 - protein, 17 aa, Homo sapiens
WISTYNGNTNYAQKLQG

SEQ ID NO: 206 - protein, 16 aa, Homo sapiens
DYYSDSSGYWDDAFDI

SEQ ID NO: 207 - protein, 108 aa, Homo sapiens
DIQLTQSPSSLSASVGDRVTITCRASQNINNYLNWYQQKPGKAPKLLLYAASSLQSGVPSRFSGSGSGTEFTLTISSLHPEDFATYYCQQSYNTPFTFGPGTKVDIKR

SEQ ID NO: 208 - protein, 125 aa, Homo sapiens
QVQLQQSGAEVKKPGASVKVSCKASGYTSTNYGISWVRQAPGQGLEWMGWISTYNGNTNYAQKLQGRVTMTTDTSTSTAYMELRSLRSDDTAVYYCARDYYSDSSGYWDDAFDIWGQGTMVTVSS

SEQ ID NO: 209 - protein, 11 aa, Homo sapiens
RASQSISSYLN

SEQ ID NO: 210 - protein, 7 aa, Homo sapiens
AASSLQS

SEQ ID NO: 211 - protein, 10 aa, Homo sapiens
QQSYSTPPWT

SEQ ID NO: 212 - protein, 10 aa, Homo sapiens
GYSFGNNGIT

SEQ ID NO: 213 - protein, 17 aa, Homo sapiens
WISTYNGNTNYAQKLQG

SEQ ID NO: 214 - protein, 16 aa, Homo sapiens
DYYSDSSGYWDDAFDI

SEQ ID NO: 215 - protein, 109 aa, Homo sapiens
DIQLTQSPSSLSASVGDRVTITCRASQSISSYLNWYQQKPGKAPKLLIYAASSLQSGVPSRFSGSGSGTDFTLTISSLQPEDFATYYCQQSYSTPPWTFGQGTKVEIKR

SEQ ID NO: 216 - protein, 125 aa, Homo sapiens
QVQLQQSGAEVKKPGASVRVSCKASGYSFGNNGITWVRQAPGQGLEWMGWISTYNGNTNYAQKLQGRVTMTTDTSTSTAYMELRSLRSDDTAVYYCARDYYSDSSGYWDDAFDIWGQGTTVTVSS

SEQ ID NO: 217 - protein, 14 aa, Homo sapiens
TGTSSDFGDYDYVS

SEQ ID NO: 218 - protein, 7 aa, Homo sapiens
DVSDRPS

SEQ ID NO: 219 - protein, 10 aa, Homo sapiens
SSFTTSSTLV

SEQ ID NO: 220 - protein, 10 aa, Homo sapiens
GYTFTGYYMH

SEQ ID NO: 221 - protein, 17 aa, Homo sapiens
WINPNSGGTNYAQKFQG

SEQ ID NO: 222 - protein, 13 aa, Homo sapiens
DGDMVYDSSGPDY

SEQ ID NO: 223 - protein, 111 aa, Homo sapiens
QSALTQPASVSGSPGQSITISCTGTSSDFGDYDYVSWYQQHPGKAPKLMIYDVSDRPSGVSNRFSGSKSGNTASLTISGLQAEDEADYFCSSFTTSSTLVFGGGTKLTVLG

SEQ ID NO: 224 - protein, 122 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDGDMVYDSSGPDYWGQGTLVTVSS

SEQ ID NO: 225 - protein, 14 aa, Homo sapiens
TGTSSDFGDYDYVS

SEQ ID NO: 226 - protein, 7 aa, Homo sapiens
DVSDRPS

SEQ ID NO: 227 - protein, 10 aa, Homo sapiens
SSLTTSSTLV

SEQ ID NO: 228 - protein, 12 aa, Homo sapiens
GGSISSSSYYWG

SEQ ID NO: 229 - protein, 16 aa, Homo sapiens
SIYYSGSTYYNPSLKS

SEQ ID NO: 230 - protein, 9 aa, Homo sapiens
HDGTDAFDI

SEQ ID NO: 231 - protein, 111 aa, Homo sapiens
QSVLTQPASVSGSPGQSITISCTGTSSDFGDYDYVSWYQQHPGKAPKLMIYDVSDRPSGVSNRFSGSKSGNTASLTISGLQAEDEADYFCSSLTTSSTLVFGGGTKLTVLG

SEQ ID NO: 232 - protein, 119 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLGSVTAADTAVYYCARHDGTDAFDIWGQGTTVTVSS

SEQ ID NO: 233 - protein, 11 aa, Homo sapiens
RTSQSISSYLN

SEQ ID NO: 234 - protein, 7 aa, Homo sapiens
AASSLQS

SEQ ID NO: 235 - protein, 9 aa, Homo sapiens
QQSYSTPFT

SEQ ID NO: 236 - protein, 10 aa, Homo sapiens
GGAFTNFGIS

SEQ ID NO: 237 - protein, 17 aa, Homo sapiens
WISTYNSETNYAQKLQG

SEQ ID NO: 238 - protein, 16 aa, Homo sapiens
DYYSDSSGYWDDAFDI

SEQ ID NO: 239 - protein, 108 aa, Homo sapiens
AIQMTQSPSSLSASVGDRVTITCRTSQSISSYLNWYQQKPGKAPKLLIYAASSLQSGVPSRFSGSGSGTDFTLTISSLQPEDFATYYCQQSYSTPFTFGPGTKVDIKR

SEQ ID NO: 240 - protein, 125 aa, Homo sapiens
EVQLVQSGAEVKKPGSSVKISCKASGGAFTNFGISWVRQAPGQGLEWMGWISTYNSETNYAQKLQGRVTMTTDTSTSTAYMELRSLRSDDTAVYYCARDYYSDSSGYWDDAFDIWGQGTLVTVSS

SEQ ID NO: 241 - protein, 13 aa, Homo sapiens
SGGSNNIGRSSVY

SEQ ID NO: 242 - protein, 7 aa, Homo sapiens
KTDQRPS

SEQ ID NO: 243 - protein, 11 aa, Homo sapiens
ATWDDSLSAVV

SEQ ID NO: 244 - protein, 12 aa, Homo sapiens
GDSVSSNSATWN

SEQ ID NO: 245 - protein, 18 aa, Homo sapiens
RTYYRSKWYNDYAVSVKS

SEQ ID NO: 246 - protein, 7 aa, Homo sapiens
GVRAFDI

SEQ ID NO: 247 - protein, 111 aa, Homo sapiens
QSVLTQPPSVSGTPGQRVTIFCSGGSNNIGRSSVYWYRQAAGTAPKLLIYKTDQRPSGVPDRFAASKSGASASLAISGLRSEDEADYHCATWDDSLSAVVFGGGTKLTVLG

SEQ ID NO: 248 - protein, 119 aa, Homo sapiens
QVQLQQSGPGLVKPSQTLSLTCAISGDSVSSNSATWNWIRQSPSRGLEWLGRTYYRSKWYNDYAVSVKSRIIINPDTSKNQFSLQLNSVTPEDTAVYYCARGVRAFDIWGQGTTVTVSS

SEQ ID NO: 249 - protein, 14 aa, Homo sapiens
TGTSSDFGDYDYVS

SEQ ID NO: 250 - protein, 7 aa, Homo sapiens
DVSDRPS

SEQ ID NO: 251 - protein, 10 aa, Homo sapiens
SSFTTSSTLV

SEQ ID NO: 252 - protein, 10 aa, Homo sapiens
GYTFRNSGIT

SEQ ID NO: 253 - protein, 17 aa, Homo sapiens
WINPNSGGAMYVDNFQG

SEQ ID NO: 254 - protein, 11 aa, Homo sapiens
GMADLIDVFDI

SEQ ID NO: 255 - protein, 116 aa, Homo sapiens; identical to SEQ ID NO: 119

SEQ ID NO: 256 - protein, 120 aa, Homo sapiens
QVQLVQSGAEVKEPGASVKVSCKASGYTFRNSGITWVRQAPGQGLEWMGWINPNSGGAMYVDNFQGRATMTRDTSINTAYMELRSLSSDDTAVYYCARGMADLIDVFDIWGQGTLVTVSS

SEQ ID NO: 257 - protein, 13 aa, Homo sapiens
TGNGGRVANNYVQ

SEQ ID NO: 258 - protein, 7 aa, Homo sapiens
EDNQRPS

SEQ ID NO: 259 - protein, 10 aa, Homo sapiens
QSYDISNQRV

SEQ ID NO: 260 - protein, 10 aa, Homo sapiens
GFNFDNYGLS

SEQ ID NO: 261 - protein, 16 aa, Homo sapiens
FIYKSVNTNYSPSLKS

SEQ ID NO: 262 - protein, 10 aa, Homo sapiens
GKVETSVVDY

SEQ ID NO: 263 - protein, 112 aa, Homo sapiens
NFMLTQPRSVSESPGKTVTISCTGNGGRVANNYVQWYQQRPGSAPTTVIYEDNQRPSGVPARFSGSIDSSSNSASLTISGLKTDDEADYYCQSYDISNQRVFGGGTKLTVLG

SEQ ID NO: 264 - protein, 118 aa, Homo sapiens
EVQLVESGGGVVRPGGSLRLSCATSGFNFDNYGLSWVRQGPGKGLEWMGFIYKSVNTNYSPSLKSRLTISMDTSKNQFSLNLASVTTADTAIYYCARGKVETSVVDYWGQGTLVTVSS

SEQ ID NO: 265 - protein, 11 aa, Homo sapiens
RASQGISTLLA

SEQ ID NO: 266 - protein, 7 aa, Homo sapiens
SASSLQS

SEQ ID NO: 267 - protein, 7 aa, Homo sapiens
QSYRAPT

SEQ ID NO: 268 - protein, 12 aa, Homo sapiens
GFSLSTRGVGVG

SEQ ID NO: 269 - protein, 16 aa, Homo sapiens
LIYWDDDKRYSPSLKS

SEQ ID NO: 270 - protein, 9 aa, Homo sapiens
QTMTGAFDI

SEQ ID NO: 271 - protein, 107 aa, Homo sapiens
DIQMTQSPSSVSASVGDRVTITCRASQGISTLLAWYQQKPGKAPKLLISSASSLQSGVPARFSGSGSGTDFTLTISSLQPEDFATYYCQQSYRAPTFGQGTKVEIKR

SEQ ID NO: 272 - protein, 119 aa, Homo sapiens
QVTLKESGPTLLKPTQTLTLTCTFSGFSLSTRGVGVGWIRQPPGQALEWLTLIYWDDDKRYSPSLKSRLTITKDTSKNQVVLTMTNMESVDTATYYCAQQTMTGAFDIWGQGTTVTVSS

SEQ ID NO: 273 - protein, 11 aa, Homo sapiens
KASQDIDDDLN

SEQ ID NO: 274 - protein, 7 aa, Homo sapiens
EATTLVP

SEQ ID NO: 275 - protein, 9 aa, Homo sapiens
LQHDNFPPT

SEQ ID NO: 276 - protein, 10 aa, Homo sapiens
GGSMNNYYWS

SEQ ID NO: 277 - protein, 16 aa, Homo sapiens
RIYSSGSTNYNPALKS

SEQ ID NO: 278 - protein, 13 aa, Homo sapiens
ASWSGTYWALFDY

SEQ ID NO: 279 - protein, 108 aa, Homo sapiens
ETTLTQSPAFMSATPGDKVNISCKASQDIDDDLNWYQQKPGEAPILIIQEATTLVPGIPPRFSGSGFGTDFTLTINSMQSEDVAYYFCLQHDNFPPTFGQGTKVEIKR

SEQ ID NO: 280 - protein, 121 aa, Homo sapiens
QVQLQESGPGLVKSSETLSLTCTVSGGSMNNYYWSWIRQPAGKGLEWMGRIYSSGSTNYNPALKSRVTMSVDTSKNQFSLNLSSVTAADTAIYYCARASWSGTYWALFDYWGQGTLVTVSS

SEQ ID NO: 281 - protein, 11 aa, Homo sapiens
RASQSISNYLN

SEQ ID NO: 282 - protein, 7 aa, Homo sapiens
AASSLQS

SEQ ID NO: 283 - protein, 9 aa, Homo sapiens
QQSYSTPFT

SEQ ID NO: 284 - protein, 10 aa, Homo sapiens
GHSFSTYGFS

SEQ ID NO: 285 - protein, 17 aa, Homo sapiens
WISTYNGNTNYAQKLQG

SEQ ID NO: 286 - protein, 16 aa, Homo sapiens
DYYSDSSGYWDDAFDI

SEQ ID NO: 287 - protein, 108 aa, Homo sapiens
DIQLAQSPSSLSASVGDRVTITCRASQSISNYLNWYQQKPGKAPKLLIYAASSLQSGVPSRFSGSGSGTDFTLTISSLQPEDFATYYCQQSYSTPFTFGPGTKVEIKR

SEQ ID NO: 288 - protein, 125 aa, Homo sapiens
EVQLVQSGNEVKRPGASVKVSCKASGHSFSTYGFSWVRQAPGQGLEWMGWISTYNGNTNYAQKLQGRVTMTTDTSTSTAYMELRSLRSDDTAVYYCARDYYSDSSGYWDDAFDIWGQGTLVTVSS

SEQ ID NO: 289 - protein, 13 aa, Homo sapiens
SGSSSNIGKNYVS

SEQ ID NO: 290 - protein, 7 aa, Homo sapiens
DNNERPS

SEQ ID NO: 291 - protein, 11 aa, Homo sapiens
ATFDTSLWAAV

SEQ ID NO: 292 - protein, 12 aa, Homo sapiens
GGSISSNSYYWG

SEQ ID NO: 293 - protein, 16 aa, Homo sapiens
SIYYSGSTYYNPSLKS

SEQ ID NO: 294 - protein, 9 aa, Homo sapiens
HDGTDAFDI

SEQ ID NO: 295 - protein, 111 aa, Homo sapiens
QSVVTQPPSVSAAPGQKVTISCSGSSSNIGKNYVSWYQQFPGTAPKLLIYDNNERPSGIPARFSGSKSGTSATLGITGLQTGDEADYYCATFDTSLWAAVFGGGTKLTVLG

SEQ ID NO: 296 - protein, 119 aa, Homo sapiens
QLQLQESGPGLVKPSETLSLTCTVSGGSISSNSYYWGWIRQPPGKGLEWIGSIYYSGSTYYNPSLKSRVTISVDTSKNQFSLKLGSVTAADTAVYYCARHDGTDAFDIWGQGTTVTVSS

SEQ ID NO: 297 - protein, 14 aa, Homo sapiens
TGTSSDVGGYNYVS

SEQ ID NO: 298 - protein, 7 aa, Homo sapiens
EVSQRPS

SEQ ID NO: 299 - protein, 9 aa, Homo sapiens
SSYAGDRDV

SEQ ID NO: 300 - protein, 10 aa, Homo sapiens
GFTFSSYSMN

SEQ ID NO: 301 - protein, 17 aa, Homo sapiens
SISSSSSYIYYADSVKG

SEQ ID NO: 302 - protein, 12 aa, Homo sapiens
GLGGWTHDAFDI

SEQ ID NO: 303 - protein, 110 aa, Homo sapiens
QSALTQPASVSGSPGQSITISCTGTSSDVGGYNYVSWYQQHPGKAPKLLIYEVSQRPSGVPDRFSGSKSGNTASLTVSGLQAEDEADYYCSSYAGDRDVFGTGTQLTVLS

SEQ ID NO: 304 - protein, 121 aa, Homo sapiens; identical to SEQ ID NO: 136

SEQ ID NO: 305 - protein, 14 aa, Homo sapiens
TGTSSDVGGYNYVS

SEQ ID NO: 306 - protein, 7 aa, Homo sapiens
DVTKRPS

SEQ ID NO: 307 - protein, 10 aa, Homo sapiens
ASYTRSTTLV

SEQ ID NO: 308 - protein, 10 aa, Homo sapiens
GFTFSSYSMN

SEQ ID NO: 309 - protein, 17 aa, Homo sapiens
SISSSSSYIYYADSVKG

SEQ ID NO: 310 - protein, 12 aa, Homo sapiens
GLGGWTHDAFDI

SEQ ID NO: 311 - protein, 111 aa, Homo sapiens
QSALTQPASVSGSPGQSITLSCTGTSSDVGGYNYVSWYQQHPGNGPKLIIYDVTKRPSGVSNRFSGSKSGNTAYLTISGLQAEDEADYYCASYTRSTTLVFGGGTKLTVLG

SEQ ID NO: 312 - protein, 121 aa, Homo sapiens
QVQLVQSGGDLVQPGGSLRLSCAASGFTFSSYSMNWVRQAPGKGLEWVSSISSSSSYIYYADSVKGRFTISRDNAKNSLYLQMNSLRAEDTAVYYCARGLGGWTHDAFDIWGQGTTVTVSS

SEQ ID NO: 313 - protein, 14 aa, Homo sapiens
TGTSSDVGGYNYVS

SEQ ID NO: 314 - protein, 7 aa, Homo sapiens
DVSKRPS

SEQ ID NO: 315 - protein, 9 aa, Homo sapiens
SSYTGRSTV

SEQ ID NO: 316 - protein, 10 aa, Homo sapiens
GFTFSSYSMN

SEQ ID NO: 317 - protein, 17 aa, Homo sapiens
SISSSSSYIYYADSVKG

SEQ ID NO: 318 - protein, 12 aa, Homo sapiens
GLGGWTHDAFDI

SEQ ID NO: 319 - protein, 110 aa, Homo sapiens
QSALTQPASVSGSPGQSITISCTGTSSDVGGYNYVSWYQQHPGKAPKLMIYDVSKRPSGVSNRFSGSKSGNTASLTISGLQAEDEADYYCSSYTGRSTVFGGGTKLTVLG

SEQ ID NO: 320 - protein, 121 aa, Homo sapiens; identical to SEQ ID NO: 96

SEQ ID NO: 321 - protein, 14 aa, Homo sapiens
TGTSSDVGGYNYVS

SEQ ID NO: 322 - protein, 7 aa, Homo sapiens
DVSNRPS

SEQ ID NO: 323 - protein, 10 aa, Homo sapiens
SSYTSSSTRV

SEQ ID NO: 324 - protein, 10 aa, Homo sapiens
GFTFSSYSMN

SEQ ID NO: 325 - protein, 17 aa, Homo sapiens
SISSSSSYIYYADSVKG

SEQ ID NO: 326 - protein, 12 aa, Homo sapiens
GLGGWTHDAFDI

SEQ ID NO: 327 - protein, 111 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGGGTKLTVLG

SEQ ID NO: 328 - protein, 121 aa, Homo sapiens; identical to SEQ ID NO: 96

SEQ ID NO: 329 - protein, 14 aa, Homo sapiens
TGSSSNIGAGYDVH

SEQ ID NO: 330 - protein, 7 aa, Homo sapiens
GNSNRPS

SEQ ID NO: 331 - protein, 11 aa, Homo sapiens
QSYDSSLSGYV

SEQ ID NO: 332 - protein, 10 aa, Homo sapiens
GFSLSSYSMN

SEQ ID NO: 333 - protein, 17 aa, Homo sapiens
SISSSSTHIYYADSLKG

SEQ ID NO: 334 - protein, 7 aa, Homo sapiens
ATIGFDY

SEQ ID NO: 335 - protein, 112 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKLTVLG

SEQ ID NO: 336 - protein, 116 aa, Homo sapiens
EVQLVETGGGLVKPGGSLRLSCEASGFSLSSYSMNWVRQAPGKGLEWVSSISSSSTHIYYADSLKGRFTISRDNAKNSLFLQMDNLRAEDTAVYYCARATIGFDYWGQGTLVTVSS

SEQ ID NO: 337 - protein, 11 aa, Homo sapiens
QGDSLRSYYAS

SEQ ID NO: 338 - protein, 7 aa, Homo sapiens
GKNNRPS

SEQ ID NO: 339 - protein, 11 aa, Homo sapiens
NSRDSSGNHLV

SEQ ID NO: 340 - protein, 10 aa, Homo sapiens
GYTFTDYYIH

SEQ ID NO: 341 - protein, 17 aa, Homo sapiens
WMNPNSGNSVSAQKFQG

SEQ ID NO: 342 - protein, 12 aa, Homo sapiens
NSEWHPWGYYDY

SEQ ID NO: 343 - protein, 110 aa, Homo sapiens
CSSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTKLTVLG

SEQ ID NO: 344 - protein, 121 aa, Homo sapiens
EVQLVQSGAEVKKPGASVKVSCKASGYTFTDYYIHWVRQAPGQGLEWMGWMNPNSGNSVSAQKFQGRVTMTRDTSINTAYMELSSLTSDDTAVYYCARNSEWHPWGYYDYWGQGTLVTVSS

SEQ ID NO: 345 - protein, 13 aa, Homo sapiens
SGSSSNIGSNTVN

SEQ ID NO: 346 - protein, 7 aa, Homo sapiens
SNNQRPS

SEQ ID NO: 347 - protein, 11 aa, Homo sapiens
AAWDDSLKSFV

SEQ ID NO: 348 - protein, 10 aa, Homo sapiens
GYTFSRYYIH

SEQ ID NO: 349 - protein, 17 aa, Homo sapiens
LINPGGGSTNYAQKFQG

SEQ ID NO: 350 - protein, 12 aa, Homo sapiens
DYGTIDARRFDF

SEQ ID NO: 351 - protein, 111 aa, Homo sapiens
QSVLTQPPSASGTPGQRVTISCSGSSSNIGSNTVNWYQQLPGTAPKLLIYSNNQRPSGVPDRFSGSKSGTSASLAIRGLQSDDEAEYYCAAWDDSLKSFVFGKGTKVTVLG

SEQ ID NO: 352 - protein, 121 aa, Homo sapiens
QVQLVQSGAEVKKPGASVKVSCKASGYTFSRYYIHWVRQAPGQGLEWMGLINPGGGSTNYAQKFQGRVTMTRDTSTNTVYLELSSLRSDDTAVYYCARDYGTIDARRFDFWGQGTLVTVSS

SEQ ID NO: 353 - protein, 13 aa, Homo sapiens
SGSSSNIGSNYVY

SEQ ID NO: 354 - protein, 7 aa, Homo sapiens
RNNQRPS

SEQ ID NO: 355 - protein, 11 aa, Homo sapiens
AAWDDSLSAWV

SEQ ID NO: 356 - protein, 10 aa, Homo sapiens
GYTFSRYYIH

SEQ ID NO: 357 - protein, 17 aa, Homo sapiens
IINTDGGTTTYAQKFQG

SEQ ID NO: 358 - protein, 12 aa, Homo sapiens
DYGTIDARRFDY

SEQ ID NO: 359 - protein, 111 aa, Homo sapiens
QSALTQPPSASGTPGQRVTISCSGSSSNIGSNYVYWYQQLPGTAPKLLIYRNNQRPSGVPDRFSGSKSGTSASLAISGLRSEDEADYYCAAWDDSLSAWVFGGGTKLTVLG

SEQ ID NO: 360 - protein, 121 aa, Homo sapiens
QVQLQQSGAEVKKPGASVKVSCKASGYTFSRYYIHWVRQAPGQGLEWMGIINTDGGTTTYAQKFQGRLTMTRDTSTSTVYMELSSLRSDDTAVYYCARDYGTIDARRFDYWGQGTLVTVSS

SEQ ID NO: 361 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 362 - protein, 5 aa, Artificial Sequence
  Peptide linker sequence
DGGGS

SEQ ID NO: 363 - protein, 5 aa, Artificial Sequence
  Peptide linker sequence
TGEKP

SEQ ID NO: 364 - protein, 4 aa, Artificial Sequence
  Peptide linker sequence
GGRR

SEQ ID NO: 365 - protein, 5 aa, Artificial Sequence
  Peptide linker sequence
GGGGS

SEQ ID NO: 366 - protein, 14 aa, Artificial Sequence
  Peptide linker sequence
EGKSSGSGSESKVD

SEQ ID NO: 367 - protein, 18 aa, Artificial Sequence
  Peptide linker sequence
KESGSVSSEQLAQFRSLD

SEQ ID NO: 368 - protein, 8 aa, Artificial Sequence
  Peptide linker sequence
GGRRGGGS

SEQ ID NO: 369 - protein, 9 aa, Artificial Sequence
  Peptide linker sequence
LRQRDGERP

SEQ ID NO: 370 - protein, 12 aa, Artificial Sequence
  Peptide linker sequence
LRQKDGGGSERP

SEQ ID NO: 371 - protein, 16 aa, Artificial Sequence
  Peptide linker sequence
LRQKDGGGSGGGSERP

SEQ ID NO: 372 - protein, 18 aa, Artificial Sequence
  Peptide linker sequence
GSTSGSGKPGSGEGSTKG

SEQ ID NO: 373 - protein, 7 aa, Artificial Sequence
  Cleavage sequence by TEV protease
  misc_feature (2)..(3): Xaa can be any naturally occurring amino acid
  misc_feature (5)..(5): Xaa can be any naturally occurring amino acid
  MISC_FEATURE (7)..(7): Xaa = Gly or Ser
EXXYXQX

SEQ ID NO: 374 - protein, 7 aa, Artificial Sequence
  Cleavage sequence by TEV protease
ENLYFQG

SEQ ID NO: 375 - protein, 7 aa, Artificial Sequence
  Cleavage sequence by TEV protease
ENLYFQS

SEQ ID NO: 376 - protein, 19 aa, Artificial Sequence
  Self-cleaving polypeptide comprising 2A site
LLNFDLLKLAGDVESNPGP

SEQ ID NO: 377 - protein, 19 aa, Artificial Sequence
  Self-cleaving polypeptide comprising 2A site
TLNFDLLKLAGDVESNPGP

SEQ ID NO: 378 - protein, 14 aa, Artificial Sequence
  Self-cleaving polypeptide comprising 2A site
LLKLAGDVESNPGP

SEQ ID NO: 379 - protein, 17 aa, Artificial Sequence
  Self-cleaving polypeptide comprising 2A site
NFDLLKLAGDVESNPGP

SEQ ID NO: 380 - protein, 20 aa, Artificial Sequence
  Self-cleaving polypeptide comprising 2A site
QLLNFDLLKLAGDVESNPGP

SEQ ID NO: 381 - protein, 24 aa, Artificial Sequence
  Self-cleaving polypeptide comprising 2A site
APVKQTLNFDLLKLAGDVESNPGP

SEQ ID NO: 382 - protein, 40 aa, Artificial Sequence
  Self-cleaving polypeptide comprising 2A site
VTELLYRMKRAETYCPRPLLAIHPTEARHKQKIVAPVKQT

SEQ ID NO: 383 - protein, 18 aa, Artificial Sequence
  Self-cleaving polypeptide comprising 2A site
LNFDLLKLAGDVESNPGP

SEQ ID NO: 384 - protein, 40 aa, Artificial Sequence
  Self-cleaving polypeptide comprising 2A site
LLAIHPTEARHKQKIVAPVKQTLNFDLLKLAGDVESNPGP

SEQ ID NO: 385 - protein, 33 aa, Artificial Sequence
  Self-cleaving polypeptide comprising 2A site
EARHKQKIVAPVKQTLNFDLLKLAGDVESNPGP

SEQ ID NO: 386 - protein, 489 aa, Artificial Sequence
  Anti-ROR1 CAR2
MALPVTALLLPLALLLHAARPAIRMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIKRGGGGSGGGGSGGGGSEVQLVESGGGLVKPGGSLRLSCAASGFTFSDYYMSWIRQAPGKGLEWVSYISDSTNTIYYADSVKGRFTVSRDNPKNSLYLQMISLRAEDTAVYYCARAVGAGEGFDHWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 387 - protein, 497 aa, Artificial Sequence
  Anti-ROR1 CAR4
MALPVTALLLPLALLLHAARPEIVMTQSPATLSVSPGERATLSCRASQSVSSNLAWYQQKPGQAPRLLIYGASTRATGIPARFSGSGSGTEFTLTISSLQSEDFAVYYCQQYNNWPPYTFGQGTKVEIKRGGGGSGGGGSGGGGSQVTLKESGPTLVKPTQTLTLTCTFSGFSLNSFGVAVGWFRQPPGKALEWLGLIYWDDDRRYFPSLEGRLSITKDASDNNVVLTMMNVDPADTATYYCARTSPMVQGIANYYAMDVWGQGTTVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 388 - protein, 489 aa, Artificial Sequence
  Anti-ROR1 CAR6
MALPVTALLLPLALLLHAARPDIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKLEIKRGGGGSGGGGSGGGGSEVQLVESGGGLVKPGGSLRLSCAASGFTFSDYYMGWVRQAPGKGLKWLSYISDRAHTIYDTDSVKGRFTISRDDAKSSLYLRMNNLRVEDTAVYYCARAVGAGEGFDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 389 - protein, 494 aa, Artificial Sequene
  Anti-ROR1 CAR15
MALPVTALLLPLALLLHAARPQSALTQPPSASGTPGQRVTISCSGSSSNIGSNYVYWYQQLPGTAPKLLIYRNNQRPSGVPDRFSGSKSGTSASLAISGLRSEDEADYYCAAWDDSLSGYVFGTGTKVTVLGGGGGSGGGGSGGGGSQMQLVQSGAEVKKPGASVKVSCKASGYTFSRYYIHWVRQAPGQGLEWMGLINPGGGSTNYAQKFQGRVTMTRDTSTNTVYLELSSLRSDDTAVYYCARDYGTIDARRFDFWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 390 - protein, 495 aa, Artificial Sequence
  Anti-ROR1 CAR50
MALPVTALLLPLALLLHAARPQSALTQPALTQPASVSGSPGQSITISCTGTSSDFGDYDYVSWYQQHPGKAPKLMIYDVSDRPSGVSNRFSGSKSGNTASLTISGLQAEDEADYFCSSFTTSSTLVFGGGTKLTVLGGGGGSGGGGSGGGGSQVQLVQSGAEVKEPGASVKVSCKASGYTFRNSGITWVRQAPGQGLEWMGWINPNSGGAMYVDNFQGRATMTRDTSINTAYMELRSLSSDDTAVYYCARGMADLIDVFDIWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 391 - protein, 485 aa, Artificial Sequence
  Anti-ROR1 CAR53
MALPVTALLLPLALLLHAARPDIQMTQSPSSVSASVGDRVTITCRASQGISTLLAWYQQKPGKAPKLLISSASSLQSGVPARFSGSGSGTDFTLTISSLQPEDFATYYCQQSYRAPTFGQGTKVEIKRGGGGSGGGGSGGGGSQVTLKESGPTLLKPTQTLTLTCTFSGFSLSTRGVGVGWIRQPPGQALEWLTLIYWDDDKRYSPSLKSRLTITKDTSKNQVVLTMTNMESVDTATYYCAQQTMTGAFDIWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 392 - protein, 488 aa, Artificial Sequence
  Anti-ROR1 CAR54
MALPVTALLLPLALLLHAARPETTLTQSPAFMSATPGDKVNISCKASQDIDDDLNWYQQKPGEAPILIIQEATTLVPGIPPRFSGSGFGTDFTLTINSMQSEDVAYYFCLQHDNFPPTFGQGTKVEIKRGGGGSGGGGSGGGGSQVQLQESGPGLVKSSETLSLTCTVSGGSMNNYYWSWIRQPAGKGLEWMGRIYSSGSTNYNPALKSRVTMSVDTSKNQFSLNLSSVTAADTAIYYCARASWSGTYWALFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 393 - protein, 490 aa, Artificial Sequence
  Anti-ROR1 CAR60
MALPVTALLLPLALLLHAARPQSALTQPASVSGSPGQSITISCTGTSSDVGGYNYVSWYQQHPGKAPKLMIYDVSKRPSGVSNRFSGSKSGNTASLTISGLQAEDEADYYCSSYTGRSTVFGGGTKLTVLGGGGGSGGGGSGGGGSEVQLVQSGGDLVQPGGSLRLSCAASGFTFSSYSMNWVRQAPGKGLEWVSSISSSSSYIYYADSVKGRFTISRDNAKNSLYLQMNSLRAEDTAVYYCARGLGGWTHDAFDIWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 394 - protein, 491 aa, Artificial Sequence
  Anti-ROR1 CAR66
MALPVTALLLPLALLLHAARPQSVLTQPPSASGTPGQRVTISCSGSSSNIGSNTVNWYQQLPGTAPKLLIYSNNQRPSGVPDRFSGSKSGTSASLAIRGLQSDDEAEYYCAAWDDSLKSFVFGKGTKVTVLGGGGGSGGGGSGGGGSQVQLVQSGAEVKKPGASVKVSCKASGYTFSRYYIHWVRQAPGQGLEWMGLINPGGGSTNYAQKFQGRVTMTRDTSTNTVYLELSSLRSDDTAVYYCARDYGTIDARRFDFWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 395 - protein, 483 aa, Artificial Sequence
  Anti-ROR1 CAR42
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSFGPGTKVDIKRGGGGSGGGGSGGGGSQVQLVQSGTEVKKPGSSVKVSCQASGGSLSSHGVSWLRQAPGQGLEWVGRIIPMFGVTDYAQKFQDRVTITADKSTSTVYMELISLGSDDTAVYFCARESRGATFEYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 396 - protein, 491 aa, Artificial Sequence
  Anti-ROR1 CAR45
MALPVTALLLPLALLLHAARPQSALTQPPSASGTPGQRVTISCPGSSSNIGSNYVYWYQQLPGTAPKLLIYRNNQRPSGVPDRFSGSKSGTSATLGITGLQTGDEADYYCGTWDSSLSAYVFGTGTKVTVLGGGGGSGGGGSGGGGSQVQLQQSGAEVKKPGASVKVSCKASGYTFSRYYIHWVRRAPGQGLEWMGLINPGGGSTNYAQKFQGRVTMTRDTSTNTVYLELSSLRSDDTAVYYCARDYGTIDARRFDFWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 397 - protein, 492 aa, Artificial Sequence
  Anti-ROR1 CAR46
MALPVTALLLPLALLLHAARPQSALTQPASVSGSPGQSITISCTGTSSDFGDYDYVSWYQQHPGKAPKLMIYDVSDRPSGVSNRFSGSKSGNTASLTISGLQAEDEADYFCSSFTTSSTLVFGGGTKLTVLGGGGGSGGGGSGGGGSQVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDGDMVYDSSGPDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 398 - protein, 4 aa, Artificial Sequence
  Predictive CDR framework region peptide
  misc_feature (3)..(3): Xaa can be any naturally occurring amino acid
FGXG

SEQ ID NO: 399 - protein, 5 aa, Artificial Sequence
  Predictive CDR framework region peptide
  misc_feature (2)..(5): Xaa can be any naturally occurring amino acid
CXXXX

SEQ ID NO: 400 - protein, 5 aa, Artificial Sequence
  Predictive CDR framework region peptide
LEWIG

SEQ ID NO: 401 - protein, 4 aa, Artificial Sequence
  Predictive CDR framework region peptide
  misc_feature (3)..(3): Xaa can be any naturally occurring amino acid
WGXG

SEQ ID NO: 402 - DNA, 10 bp, Artificial Sequence
  Consensus Kosak sequence
gccrccatgg